SEQUENCE LISTING (476 sequences)

SEQ ID NO: 1 - protein, 12 aa, Mus musculus
QATCDACAPGYT

SEQ ID NO: 2 - protein, 9 aa, Mus musculus
QAFENGVRM

SEQ ID NO: 3 - protein, 11 aa, Mus musculus
QKCVWGHNKER

SEQ ID NO: 4 - protein, 19 aa, Mus musculus
QSTEVDRSVDRLTFGDLLP

SEQ ID NO: 5 - protein, 19 aa, Mus musculus
QNEASPTPRCMMHSRAMGR

SEQ ID NO: 6 - protein, 5 aa, Mus musculus
QNASR

SEQ ID NO: 7 - protein, 19 aa, Mus musculus
QDCRVNNGGCLHYCLEESN

SEQ ID NO: 8 - protein, 7 aa, Mus musculus
QSDRIKD

SEQ ID NO: 9 - protein, 16 aa, Mus musculus
QGCVNTKGSYECVCPP

SEQ ID NO: 10 - protein, 6 aa, Mus musculus
QFFGLM

SEQ ID NO: 11 - protein, 16 aa, Mus musculus
QISHKRHKTDSFVGLM

SEQ ID NO: 12 - protein, 6 aa, Mus musculus
QFYGLM

SEQ ID NO: 13 - protein, 4 aa, Mus musculus
QGLV

SEQ ID NO: 14 - protein, 10 aa, Mus musculus
QGPPGPAGEE

SEQ ID NO: 15 - protein, 5 aa, Mus musculus
QVTVA

SEQ ID NO: 16 - protein, 9 aa, Mus musculus
QHSHASRSR

SEQ ID NO: 17 - protein, 9 aa, Mus musculus
QGSHADRSR

SEQ ID NO: 18 - protein, 16 aa, Mus musculus
QLYSGGGKVGYLVYAG

SEQ ID NO: 19 - protein, 20 aa, Mus musculus
QFAYRHCNNPAPRNSGRYCT

SEQ ID NO: 20 - skipped

SEQ ID NO: 21 - protein, 11 aa, Mus musculus
QGNPSRSHGRG

SEQ ID NO: 22 - protein, 15 aa, Mus musculus
QCVSEGLSFHCLCPE

SEQ ID NO: 23 - protein, 6 aa, Mus musculus
QKRHFC

SEQ ID NO: 24 - protein, 8 aa, Mus musculus
QSALVGKN

SEQ ID NO: 25 - protein, 5 aa, Mus musculus
QDPSG

SEQ ID NO: 26 - protein, 5 aa, Mus musculus
QASHH

SEQ ID NO: 27 - protein, 20 aa, Mus musculus
QFEENWYNTYSSNIYKHGDT

SEQ ID NO: 28 - protein, 15 aa, Mus musculus
QRKCIVEILVEPKDI

SEQ ID NO: 29 - protein, 13 aa, Mus musculus
QVEEVRLRPAVEW

SEQ ID NO: 30 - protein, 4 aa, Mus musculus
QVYT

SEQ ID NO: 31 - protein, 13 aa, Mus musculus
QGNDGDPGPVGPA

SEQ ID NO: 32 - protein, 5 aa, Mus musculus
QLGPP

SEQ ID NO: 33 - protein, 20 aa, Mus musculus
QLLLPKSHSWGIVLPLGELE

SEQ ID NO: 34 - protein, 18 aa, Mus musculus
QRRLDFLAKHILHCKAKY

SEQ ID NO: 35 - protein, 7 aa, Mus musculus
QVVFEKC

SEQ ID NO: 36 - protein, 19 aa, Mus musculus
QIDLLAPTHLDNGVDKTSR

SEQ ID NO: 37 - skipped

SEQ ID NO: 38 - protein, 7 aa, Mus musculus
QKWKYNR

SEQ ID NO: 39 - protein, 5 aa, Mus musculus
QWSPS

SEQ ID NO: 40 - protein, 15 aa, Mus musculus
QFLDKGSENSFIDLG

SEQ ID NO: 41 - protein, 7 aa, Mus musculus
QVTYVRS

SEQ ID NO: 42 - protein, 10 aa, Mus musculus
QHWSYGLRPG

SEQ ID NO: 43 - protein, 7 aa, Mus musculus
QHWKPAL

SEQ ID NO: 44 - protein, 9 aa, Mus musculus
QCRCRPNVA

SEQ ID NO: 45 - protein, 20 aa, Mus musculus
QFEENWYNTYSSNLYKHVDT

SEQ ID NO: 46 - protein, 18 aa, Mus musculus
QREKDLSTYNWNSFGLRY

SEQ ID NO: 47 - protein, 20 aa, Mus musculus
QTPEILAALPESAPAVRPTC

SEQ ID NO: 48 - protein, 11 aa, Mus musculus
QGSLSFPLLER

SEQ ID NO: 49 - protein, 12 aa, Mus musculus
QGPIGPPGPSGV

SEQ ID NO: 50 - protein, 20 aa, Mus musculus
QSLDPSHRISDRDYMGWMDF

SEQ ID NO: 51 - protein, 7 aa, Mus musculus
QDAALGW

SEQ ID NO: 52 - skipped

SEQ ID NO: 53 - protein, 18 aa, Mus musculus
QDGRAECKCPAGEDWWYM

SEQ ID NO: 54 - protein, 12 aa, Mus musculus
QRCLPEFENAAF

SEQ ID NO: 55 - protein, 8 aa, Mus musculus
QRCDDADP

SEQ ID NO: 56 - protein, 6 aa, Mus musculus
QPGKAS

SEQ ID NO: 57 - protein, 10 aa, Mus musculus
QGPPGPTGKP

SEQ ID NO: 58 - protein, 17 aa, Mus musculus
QPGPPGPSGEAGPPGPP

SEQ ID NO: 59 - skipped

SEQ ID NO: 60 - protein, 11 aa, Mus musculus
QGPRGPLLSMP

SEQ ID NO: 61 - protein, 12 aa, Mus musculus
QMEPGVGILTSL

SEQ ID NO: 62 - protein, 17 aa, Mus musculus
QRPRMEEEEEAYGWMDF

SEQ ID NO: 63 - protein, 5 aa, Mus musculus
QHEEF

SEQ ID NO: 64 - skipped

SEQ ID NO: 65 - protein, 9 aa, Mus musculus
QSGVYDLRL

SEQ ID NO: 66 - protein, 4 aa, Mus musculus
QPLG

SEQ ID NO: 67 - protein, 20 aa, Mus musculus
QFSSRDCTRPVPRNGGKYCE

SEQ ID NO: 68 - protein, 4 aa, Mus musculus
QALY

SEQ ID NO: 69 - protein, 4 aa, Mus musculus
QKLY

SEQ ID NO: 70 - protein, 8 aa, Mus musculus
QYSSCTSD

SEQ ID NO: 71 - protein, 8 aa, Mus musculus
QLLDATET

SEQ ID NO: 72 - skipped

SEQ ID NO: 73 - protein, 20 aa, Mus musculus
QCSRCATGYYGFPYCKPCNC

SEQ ID NO: 74 - protein, 8 aa, Mus musculus
QDEGILPS

SEQ ID NO: 75 - protein, 14 aa, Mus musculus
QAAKEFIAWLVKGR

SEQ ID NO: 76 - protein, 7 aa, Mus musculus
QPKFSFC

SEQ ID NO: 77 - protein, 13 aa, Mus musculus
QAAAASPENSRGK

SEQ ID NO: 78 - protein, 10 aa, Mus musculus
QFGEMIRLKT

SEQ ID NO: 79 - protein, 6 aa, Mus musculus
QGPVSS

SEQ ID NO: 80 - protein, 17 aa, Mus musculus
QEKREIGDEENSAKFPI

SEQ ID NO: 81 - protein, 20 aa, Mus musculus
QKGEKGMEGEKGSRGDVGSE

SEQ ID NO: 82 - protein, 11 aa, Mus musculus
QKSVWHGSDPS

SEQ ID NO: 83 - protein, 9 aa, Mus musculus
QPAVSWSKD

SEQ ID NO: 84 - protein, 5 aa, Mus musculus
QLVTE

SEQ ID NO: 85 - protein, 4 aa, Mus musculus
QVSA

SEQ ID NO: 86 - protein, 4 aa, Mus musculus
QLRP

SEQ ID NO: 87 - protein, 7 aa, Mus musculus
QYPEVGM

SEQ ID NO: 88 - protein, 19 aa, Mus musculus
QSRPGCYGDRSSLPGVRSY

SEQ ID NO: 89 - protein, 11 aa, Mus musculus
QSPGSSAAPGP

SEQ ID NO: 90 - protein, 12 aa, Mus musculus
QMAVKKYLAAVL

SEQ ID NO: 91 - protein, 6 aa, Mus musculus
QRVKNK

SEQ ID NO: 92 - protein, 7 aa, Mus musculus
QGPRDTP

SEQ ID NO: 93 - protein, 16 aa, Mus musculus
QFAEMKLRPSNFRNLE

SEQ ID NO: 94 - protein, 18 aa, Mus musculus
QERRRAKRRKRKKEVPFW

SEQ ID NO: 95 - protein, 4 aa, Mus musculus
QRRR

SEQ ID NO: 96 - protein, 13 aa, Mus musculus
QLLPGMLCAGYPE

SEQ ID NO: 97 - protein, 8 aa, Mus musculus
QASPPRRL

SEQ ID NO: 98 - protein, 4 aa, Mus musculus
QVEA

SEQ ID NO: 99 - protein, 17 aa, Mus musculus
QGCGVVSSGSSLYFSKA

SEQ ID NO: 100 - protein, 20 aa, Mus musculus
QVGTGCGTLNDGRSLYFNGL

SEQ ID NO: 101 - protein, 5 aa, Mus musculus
QNGNA

SEQ ID NO: 102 - protein, 7 aa, Mus musculus
QCPDGFV

SEQ ID NO: 103 - protein, 6 aa, Mus musculus
QNCPRG

SEQ ID NO: 104 - protein, 8 aa, Mus musculus
QEPMKKCT

SEQ ID NO: 105 - protein, 15 aa, Mus musculus
QDSLFYSSNEFEGYP

SEQ ID NO: 106 - protein, 15 aa, Mus musculus
QSLRVSCTYDALKHW

SEQ ID NO: 107 - skipped

SEQ ID NO: 108 - protein, 14 aa, Mus musculus
QLRRYINTLTRPRY

SEQ ID NO: 109 - protein, 5 aa, Mus musculus
QVEVL

SEQ ID NO: 110 - protein, 12 aa, Mus musculus
QESDASNFLKRR

SEQ ID NO: 111 - protein, 10 aa, Mus musculus
QSSDCPCVHA

SEQ ID NO: 112 - protein, 9 aa, Mus musculus
QDCPVCEVA

SEQ ID NO: 113 - protein, 20 aa, Mus musculus
QWGGCGDNLKYSSKFVKEFL

SEQ ID NO: 114 - protein, 20 aa, Mus musculus
QGDPNCKIDLSFLIDGSTSI

SEQ ID NO: 115 - protein, 20 aa, Mus musculus
QRKPRPLDPGEGSKDTDSSA

SEQ ID NO: 116 - protein, 15 aa, Mus musculus
QSRTSSYSGEYGGKG

SEQ ID NO: 117 - protein, 7 aa, Mus musculus
QPPTRFC

SEQ ID NO: 118 - protein, 5 aa, Mus musculus
QNNEA

SEQ ID NO: 119 - protein, 9 aa, Mus musculus
QLSVERGGW

SEQ ID NO: 120 - protein, 4 aa, Mus musculus
QHMI

SEQ ID NO: 121 - protein, 9 aa, Mus musculus
QCRCKPGVV

SEQ ID NO: 122 - protein, 6 aa, Mus musculus
QVGCLH

SEQ ID NO: 123 - protein, 14 aa, Mus musculus
QPLKTPGKKKKGKP

SEQ ID NO: 124 - protein, 13 aa, Mus musculus
QKRDMHDFFVGLM

SEQ ID NO: 125 - protein, 10 aa, Mus musculus
QFNAFSYHFR

SEQ ID NO: 126 - protein, 12 aa, Mus musculus
QISAKKYLESLI

SEQ ID NO: 127 - protein, 13 aa, Mus musculus
QMAVKKYLNSILN

SEQ ID NO: 128 - protein, 4 aa, Mus musculus
QESC

SEQ ID NO: 129 - skipped

SEQ ID NO: 130 - protein, 6 aa, Mus musculus
QILAHV

SEQ ID NO: 131 - protein, 4 aa, Mus musculus
QLAD

SEQ ID NO: 132 - protein, 7 aa, Mus musculus
QTLHLGT

SEQ ID NO: 133 - protein, 7 aa, Mus musculus
QYYYDGL

SEQ ID NO: 134 - protein, 4 aa, Mus musculus
QSEI

SEQ ID NO: 135 - protein, 20 aa, Mus musculus
QYTMRECDNPVPKNGGKYCE

SEQ ID NO: 136 - protein, 12 aa, Mus musculus
QSKGYFLFRPRN

SEQ ID NO: 137 - protein, 19 aa, Mus musculus
QTNCPKKDWKKPECTIKPN

SEQ ID NO: 138 - skipped

SEQ ID NO: 139 - protein, 6 aa, Mus musculus
QLVRPA

SEQ ID NO: 140 - skipped

SEQ ID NO: 141 - protein, 12 aa, Mus musculus
QHDGTGNDCESI

SEQ ID NO: 142 - protein, 8 aa, Mus musculus
QYGEPREG

SEQ ID NO: 143 - protein, 10 aa, Mus musculus
QSCGPGRGPV

SEQ ID NO: 144 - protein, 10 aa, Mus musculus
QLLGESDFAL

SEQ ID NO: 145 - protein, 8 aa, Mus musculus
QRRNPENG

SEQ ID NO: 146 - skipped

SEQ ID NO: 147 - protein, 13 aa, Mus musculus
QCICPPGFVLSDN

SEQ ID NO: 148 - protein, 6 aa, Mus musculus
QKKKEE

SEQ ID NO: 149 - protein, 16 aa, Mus musculus
QWRRARHNYNDLCPPI

SEQ ID NO: 150 - protein, 17 aa, Mus musculus
QPFPRAEGMPEAILKVK

SEQ ID NO: 151 - protein, 4 aa, Mus musculus
QSPG

SEQ ID NO: 152 - protein, 20 aa, Mus musculus
QDCDFKDAEEAATGECTATV

SEQ ID NO: 153 - protein, 11 aa, Mus musculus
QCYSGYTLAED

SEQ ID NO: 154 - protein, 16 aa, Mus musculus
QHFFHLRNDFAYIEVD

SEQ ID NO: 155 - protein, 11 aa, Mus musculus
QLRSYSETIEK

SEQ ID NO: 156 - protein, 12 aa, Mus musculus
QPVYKPYLTLCA

SEQ ID NO: 157 - protein, 14 aa, Mus musculus
QHLIYKDCSDHYVL

SEQ ID NO: 158 - protein, 8 aa, Mus musculus
QLCNIPVT

SEQ ID NO: 159 - protein, 5 aa, Mus musculus
QPLYH

SEQ ID NO: 160 - protein, 19 aa, Mus musculus
QLADDGLVALDWVIGPCAR

SEQ ID NO: 161 - protein, 10 aa, Mus musculus
QGPPGPSGEE

SEQ ID NO: 162 - protein, 10 aa, Mus musculus
QGAPGPAGEE

SEQ ID NO: 163 - protein, 4 aa, Mus musculus
QLFE

SEQ ID NO: 164 - protein, 17 aa, Mus musculus
QTGSPGPTGEPGPSGPP

SEQ ID NO: 165 - protein, 4 aa, Mus musculus
QGAP

SEQ ID NO: 166 - protein, 10 aa, Mus musculus
QGPIGPPGEE

SEQ ID NO: 167 - protein, 17 aa, Mus musculus
QRGEPGSRGPVGPPGRA

SEQ ID NO: 168 - protein, 10 aa, Mus musculus
QGAKGDSGRP

SEQ ID NO: 169 - protein, 7 aa, Mus musculus
QGPKGEK

SEQ ID NO: 170 - protein, 19 aa, Mus musculus
QGEDGNPGRGGEKGAKGIR

SEQ ID NO: 171 - protein, 4 aa, Mus musculus
QGPP

SEQ ID NO: 172 - protein, 7 aa, Mus musculus
QGDHGIP

SEQ ID NO: 173 - protein, 4 aa, Mus musculus
QGNP

SEQ ID NO: 174 - protein, 12 aa, Mus musculus
QGKEAIKATSKL

SEQ ID NO: 175 - protein, 16 aa, Mus musculus
QGLGGPPGLPGSAGLP

SEQ ID NO: 176 - protein, 10 aa, Mus musculus
QASPYLFEAT

SEQ ID NO: 177 - protein, 4 aa, Mus musculus
QPSG

SEQ ID NO: 178 - protein, 7 aa, Mus musculus
QGVKGHP

SEQ ID NO: 179 - protein, 8 aa, Mus musculus
QIGNTGPK

SEQ ID NO: 180 - protein, 5 aa, Mus musculus
QVLDT

SEQ ID NO: 181 - protein, 19 aa, Mus musculus
QILRLKPLTGEGTAGGGGG

SEQ ID NO: 182 - protein, 12 aa, Mus musculus
QLELRLRVAAGR

SEQ ID NO: 183 - protein, 12 aa, Mus musculus
QPPPLDLRSLGF

SEQ ID NO: 184 - skipped

SEQ ID NO: 185 - protein, 10 aa, Mus musculus
QGPKGEKGAI

SEQ ID NO: 186 - protein, 17 aa, Mus musculus
QVYAPEVSPESIMECAT

SEQ ID NO: 187 - protein, 5 aa, Mus musculus
QGHAG

SEQ ID NO: 188 - protein, 4 aa, Mus musculus
QHFC

SEQ ID NO: 189 - protein, 9 aa, Mus musculus
QLGPSGHDA

SEQ ID NO: 190 - protein, 10 aa, Mus musculus
QGLTGPPGKA

SEQ ID NO: 191 - protein, 17 aa, Mus musculus
QPGSPGPTGENGPPGPL

SEQ ID NO: 192 - protein, 16 aa, Mus musculus
QVVFSETVITSVGDEE

SEQ ID NO: 193 - protein, 13 aa, Mus musculus
QFRKKWNKWALSR

SEQ ID NO: 194 - skipped

SEQ ID NO: 195 - protein, 20 aa, Mus musculus
QNTDYEAEKALKNPKYKDRA

SEQ ID NO: 196 - protein, 7 aa, Mus musculus
QNPDAPE

SEQ ID NO: 197 - protein, 15 aa, Mus musculus
QTVPYGLSNYRESLR

SEQ ID NO: 198 - protein, 11 aa, Mus musculus
QYLELDVNIST

SEQ ID NO: 199 - protein, 5 aa, Mus musculus
QEANP

SEQ ID NO: 200 - protein, 8 aa, Mus musculus
QECCPPEL

SEQ ID NO: 201 - protein, 4 aa, Mus musculus
QAGL

SEQ ID NO: 202 - protein, 15 aa, Mus musculus
QCYNFGWYPESPICE

SEQ ID NO: 203 - protein, 15 aa, Mus musculus
QEATGRAVSTAHPPS

SEQ ID NO: 204 - protein, 19 aa, Mus musculus
QVVPVDGSHREVRVSGLDP

SEQ ID NO: 205 - protein, 15 aa, Mus musculus
QPGRKYKFLLYGLTG

SEQ ID NO: 206 - protein, 9 aa, Mus musculus
QAEAACRPS

SEQ ID NO: 207 - protein, 10 aa, Mus musculus
QFGEMIWLKT

SEQ ID NO: 208 - protein, 7 aa, Mus musculus
QMMAMLM

SEQ ID NO: 209 - protein, 10 aa, Mus musculus
QPVTPLELFE

SEQ ID NO: 210 - protein, 13 aa, Mus musculus
QLLPGMLCAGYPA

SEQ ID NO: 211 - protein, 5 aa, Mus musculus
QPGST

SEQ ID NO: 212 - protein, 18 aa, Mus musculus
QTNTIKGLNGRPGSPGSM

SEQ ID NO: 213 - protein, 7 aa, Mus musculus
QLFGLKD

SEQ ID NO: 214 - protein, 6 aa, Mus musculus
QKLTAK

SEQ ID NO: 215 - protein, 18 aa, Mus musculus
QHHHTACSHNSSSSDCED

SEQ ID NO: 216 - protein, 11 aa, Mus musculus
QAMVSDRNHTD

SEQ ID NO: 217 - protein, 20 aa, Homo sapiens
QCTRCATGHYGFPRCKPCSC

SEQ ID NO: 218 - protein, 4 aa, Homo sapiens
QHMV

SEQ ID NO: 219 - protein, 9 aa, Homo sapiens
QCLCKPGVV

SEQ ID NO: 220 - protein, 12 aa, Homo sapiens
QATCDACAPGYT

SEQ ID NO: 221 - protein, 9 aa, Homo sapiens
QAFENGVRM

SEQ ID NO: 222 - skipped

SEQ ID NO: 223 - protein, 10 aa, Homo sapiens
QCVWGHNKER

SEQ ID NO: 224 - protein, 13 aa, Homo sapiens
QILPGMLCAGYPE

SEQ ID NO: 225 - protein, 11 aa, Homo sapiens
QAGITSFGFGC

SEQ ID NO: 226 - protein, 5 aa, Homo sapiens
QGHAG

SEQ ID NO: 227 - protein, 12 aa, Homo sapiens
QSRGYFLFRPRN

SEQ ID NO: 228 - protein, 19 aa, Homo sapiens
QLLDEGLCVESTECPCVHS

SEQ ID NO: 229 - protein, 15 aa, Homo sapiens
QTCVDPEDCPVCEVA

SEQ ID NO: 230 - protein, 17 aa, Homo sapiens
QPGPPGPSGEAGPPGPP

SEQ ID NO: 231 - protein, 4 aa, Homo sapiens
QASC

SEQ ID NO: 232 - protein, 14 aa, Homo sapiens
QVLLGGAAGLVPEL

SEQ ID NO: 233 - protein, 14 aa, Homo sapiens
QTDTAPSPSYHLLP

SEQ ID NO: 234 - protein, 10 aa, Homo sapiens
QGPPGPSGEE

SEQ ID NO: 235 - protein, 10 aa, Homo sapiens
QGPPGPAGKP

SEQ ID NO: 236 - protein, 17 aa, Homo sapiens
QTGSPGPTGEPGPSGPP

SEQ ID NO: 237 - protein, 4 aa, Homo sapiens
QAGL

SEQ ID NO: 238 - protein, 8 aa, Homo sapiens
QSLKPEPF

SEQ ID NO: 239 - protein, 8 aa, Homo sapiens
QNGGRYCL

SEQ ID NO: 240 - protein, 14 aa, Homo sapiens
QGATYPVSLVAFKG

SEQ ID NO: 241 - protein, 9 aa, Homo sapiens
QPLPSPGVG

SEQ ID NO: 242 - protein, 6 aa, Homo sapiens
QFVGHP

SEQ ID NO: 243 - protein, 16 aa, Homo sapiens
QLLLDKHMGMKRYEVA

SEQ ID NO: 244 - protein, 16 aa, Homo sapiens
QFAEMKLRPSNFRNLE

SEQ ID NO: 245 - protein, 8 aa, Homo sapiens
QLCNIPVT

SEQ ID NO: 246 - protein, 17 aa, Homo sapiens
QVTGALRSGRGKMLLFS

SEQ ID NO: 247 - protein, 20 aa, Homo sapiens
QFEENWYNTYSSNLYKHVDT

SEQ ID NO: 248 - protein, 20 aa, Homo sapiens
QCETSMDGEAGLCWCVYPWN

SEQ ID NO: 249 - protein, 13 aa, Homo sapiens
QVEEVRIRPAVGW

SEQ ID NO: 250 - skipped

SEQ ID NO: 251 - protein, 20 aa, Homo sapiens
QPLTENPRKYVMGHFRWDRF

SEQ ID NO: 252 - protein, 10 aa, Homo sapiens
QGLTGPPGKA

SEQ ID NO: 253 - protein, 17 aa, Homo sapiens
QPGSPGPTGENGPPGTL

SEQ ID NO: 254 - protein, 20 aa, Homo sapiens
QFSSRDCTRPVTRNGGKYCE

SEQ ID NO: 255 - protein, 19 aa, Homo sapiens
QILRLKPLTGEGTAGGGGG

SEQ ID NO: 256 - protein, 16 aa, Homo sapiens
QKNMTALEKKKEAVES

SEQ ID NO: 257 - protein, 6 aa, Homo sapiens
QGPVGS

SEQ ID NO: 258 - protein, 4 aa, Homo sapiens
QLFE

SEQ ID NO: 259 - protein, 12 aa, Homo sapiens
QVMAELSPAAKT

SEQ ID NO: 260 - protein, 5 aa, Homo sapiens
QMLFR

SEQ ID NO: 261 - protein, 12 aa, Homo sapiens
QGPTGPSGPPGV

SEQ ID NO: 262 - protein, 6 aa, Homo sapiens
QLSWEW

SEQ ID NO: 263 - protein, 20 aa, Homo sapiens
QFSSRDCTRPVPRNGGKYCE

SEQ ID NO: 264 - protein, 5 aa, Homo sapiens
QSGDA

SEQ ID NO: 265 - protein, 15 aa, Homo sapiens
QTVPYGLSNYRGSFR

SEQ ID NO: 266 - protein, 16 aa, Homo sapiens
QGLGGPPGLPGSAGLP

SEQ ID NO: 267 - skipped

SEQ ID NO: 268 - protein, 8 aa, Homo sapiens
QRAVPEGF

SEQ ID NO: 269 - protein, 20 aa, Homo sapiens
QYTMRECDNPVPKNGGKYCE

SEQ ID NO: 270 - protein, 13 aa, Homo sapiens
QEVLWGSPAASDD

SEQ ID NO: 271 - protein, 7 aa, Homo sapiens
QDVDECA

SEQ ID NO: 272 - protein, 10 aa, Homo sapiens
QGPPGPTGKP

SEQ ID NO: 273 - protein, 19 aa, Homo sapiens
QSRPGCYGDRSSLPGVRSY

SEQ ID NO: 274 - protein, 6 aa, Homo sapiens
QFFGLM

SEQ ID NO: 275 - protein, 16 aa, Homo sapiens
QISHKRHKTDSFVGLM

SEQ ID NO: 276 - protein, 9 aa, Homo sapiens
QVDINGFTT

SEQ ID NO: 277 - protein, 17 aa, Homo sapiens
QPGSPGPTGENGPPGPL

SEQ ID NO: 278 - protein, 8 aa, Homo sapiens
QEVCFAEE

SEQ ID NO: 279 - protein, 20 aa, Homo sapiens
QELGPPALVAPRAFAHITMA

SEQ ID NO: 280 - protein, 17 aa, Homo sapiens
QRGDPGSRGPIGPPGRA

SEQ ID NO: 281 - protein, 5 aa, Homo sapiens
QWSPS

SEQ ID NO: 282 - protein, 15 aa, Homo sapiens
QYVVLLRRSHGDRSR

SEQ ID NO: 283 - protein, 14 aa, Homo sapiens
QPLKTPGKKKKGKP

SEQ ID NO: 284 - protein, 17 aa, Homo sapiens
QGCGVISSGSSLYFSKA

SEQ ID NO: 285 - protein, 20 aa, Homo sapiens
QVGTGCGTLNDGKSLYFNGP

SEQ ID NO: 286 - protein, 10 aa, Homo sapiens
QLKEYVLTDG

SEQ ID NO: 287 - protein, 16 aa, Homo sapiens
QMVFSETVITSVGDEE

SEQ ID NO: 288 - skipped

SEQ ID NO: 289 - protein, 5 aa, Homo sapiens
QKPYS

SEQ ID NO: 290 - protein, 12 aa, Homo sapiens
QESDASNFLKRR

SEQ ID NO: 291 - protein, 13 aa, Homo sapiens
QAAAANPENSRGK

SEQ ID NO: 292 - protein, 18 aa, Homo sapiens
QTSMDGEAGLCWCVYPWN

SEQ ID NO: 293 - protein, 5 aa, Homo sapiens
QNNEA

SEQ ID NO: 294 - protein, 4 aa, Homo sapiens
QGEP

SEQ ID NO: 295 - protein, 20 aa, Homo sapiens
QPLTENPRKYVMGHFRWDRL

SEQ ID NO: 296 - protein, 4 aa, Homo sapiens
QHFC

SEQ ID NO: 297 - protein, 20 aa, Homo sapiens
QNLDPSHRISDRDYMGWMDF

SEQ ID NO: 298 - protein, 16 aa, Homo sapiens
QKGIPGDPAFGHLGPP

SEQ ID NO: 299 - protein, 11 aa, Homo sapiens
QGSGGGAGRGH

SEQ ID NO: 300 - protein, 18 aa, Homo sapiens
QAAKKNGKGNVCHRKKHH

SEQ ID NO: 301 - protein, 7 aa, Homo sapiens
QEVDECA

SEQ ID NO: 302 - protein, 12 aa, Homo sapiens
QPPPPDLRSLGF

SEQ ID NO: 303 - protein, 17 aa, Homo sapiens
QGPWLEEEEEAYGWMDF

SEQ ID NO: 304-305 - skipped (2 entries)

SEQ ID NO: 306 - protein, 18 aa, Homo sapiens
QVGPPGAACRRAYAAFSV

SEQ ID NO: 307 - protein, 6 aa, Homo sapiens
QGCLRF

SEQ ID NO: 308 - protein, 16 aa, Homo sapiens
QVPSVEDLSEDCMFGN

SEQ ID NO: 309 - protein, 20 aa, Homo sapiens
QWGGCGDNLKYSSKFVKEFL

SEQ ID NO: 310 - protein, 9 aa, Homo sapiens
QRSHGDRSR

SEQ ID NO: 311 - protein, 15 aa, Homo sapiens
QALRKGVRRTRPSRK

SEQ ID NO: 312 - protein, 4 aa, Homo sapiens
QRRR

SEQ ID NO: 313 - protein, 6 aa, Homo sapiens
QELTLV

SEQ ID NO: 314 - protein, 18 aa, Homo sapiens
QACTCPVGVACDSVSGEC

SEQ ID NO: 315 - protein, 8 aa, Homo sapiens
QINGTGLL

SEQ ID NO: 316 - protein, 6 aa, Homo sapiens
QNCPRG

SEQ ID NO: 317 - protein, 16 aa, Homo sapiens
QVTFIHLKCDSSRKGK

SEQ ID NO: 318 - protein, 17 aa, Homo sapiens
QNATNNGCAMASRSDGR

SEQ ID NO: 319 - skipped

SEQ ID NO: 320 - protein, 5 aa, Homo sapiens
QNASR

SEQ ID NO: 321 - protein, 18 aa, Homo sapiens
QYAADLRRYINMLTRPRY

SEQ ID NO: 322 - protein, 7 aa, Homo sapiens
QYPEVGT

SEQ ID NO: 323 - protein, 5 aa, Homo sapiens
QAGYM

SEQ ID NO: 324 - protein, 19 aa, Homo sapiens
QATDVDRSVDRFTFRALLP

SEQ ID NO: 325 - protein, 16 aa, Homo sapiens
QGCVNTKGSYECVCPP

SEQ ID NO: 326 - protein, 4 aa, Homo sapiens
QGLL

SEQ ID NO: 327 - protein, 6 aa, Homo sapiens
QPFLVG

SEQ ID NO: 328 - protein, 4 aa, Homo sapiens
QVLT

SEQ ID NO: 329 - protein, 20 aa, Homo sapiens
QDCEYKDAAKAATGECTATV

SEQ ID NO: 330 - protein, 18 aa, Homo sapiens
QREKDLPNYNWNSFGLRF

SEQ ID NO: 331 - protein, 16 aa, Homo sapiens
QNEILTIHFTGHSFIY

SEQ ID NO: 332 - protein, 9 aa, Homo sapiens
QCPCHGEVS

SEQ ID NO: 333 - protein, 9 aa, Homo sapiens
QCPCKLGYG

SEQ ID NO: 334 - protein, 4 aa, Homo sapiens
QVSA

SEQ ID NO: 335 - protein, 11 aa, Homo sapiens
QALATPNKEEH

SEQ ID NO: 336 - protein, 16 aa, Homo sapiens
QAALPLLSNAECKKSW

SEQ ID NO: 337 - protein, 13 aa, Homo sapiens
QLMIIKNSTYREI

SEQ ID NO: 338 - protein, 8 aa, Homo sapiens
QRRSPENS

SEQ ID NO: 339-340 - skipped (2 entries)

SEQ ID NO: 341 - protein, 16 aa, Homo sapiens
QSAPPKENPSERKTNE

SEQ ID NO: 342 - protein, 14 aa, Homo sapiens
QHLIYKDCSDYYAI

SEQ ID NO: 343 - protein, 6 aa, Homo sapiens
QNCPLG

SEQ ID NO: 344 - protein, 15 aa, Homo sapiens
QAPGRGPLGLRLMMP

SEQ ID NO: 345 - protein, 15 aa, Homo sapiens
QSPKKSVADLLGSFE

SEQ ID NO: 346 - protein, 9 aa, Homo sapiens
QCHCVPGVA

SEQ ID NO: 347 - protein, 18 aa, Homo sapiens
QNVTPLTFEEVPYSELEN

SEQ ID NO: 348 - protein, 4 aa, Homo sapiens
QRFS

SEQ ID NO: 349 - protein, 10 aa, Homo sapiens
QDEELASTHP

SEQ ID NO: 350 - protein, 16 aa, Homo sapiens
QLYSGGGKVGYLVYAG

SEQ ID NO: 351 - protein, 20 aa, Homo sapiens
QFAYRHCNNPAPRNNGRYCT

SEQ ID NO: 352 - protein, 6 aa, Homo sapiens
QSLAVN

SEQ ID NO: 353 - protein, 10 aa, Homo sapiens
QGDPHYTTFD

SEQ ID NO: 354 - protein, 12 aa, Homo sapiens
QGSGDPHYVSFD

SEQ ID NO: 355 - protein, 12 aa, Homo sapiens
QASGDPHYTTFD

SEQ ID NO: 356 - protein, 16 aa, Homo sapiens
QPGNGNEGSVTGSCYC

SEQ ID NO: 357 - protein, 12 aa, Homo sapiens
QNISNCTYADRP

SEQ ID NO: 358 - protein, 9 aa, Homo sapiens
QLGPSGHDA

SEQ ID NO: 359 - protein, 15 aa, Homo sapiens
QRKCIIDILTEPKDI

SEQ ID NO: 360 - protein, 10 aa, Homo sapiens
QHWSHGWYPG

SEQ ID NO: 361 - protein, 13 aa, Homo sapiens
QCLCPPGFVLSDN

SEQ ID NO: 362 - protein, 10 aa, Homo sapiens
QHWSYGLRPG

SEQ ID NO: 363 - protein, 16 aa, Homo sapiens
QWRRARHNYNDLCPPI

SEQ ID NO: 364 - protein, 5 aa, Homo sapiens
QFEKC

SEQ ID NO: 365 - skipped

SEQ ID NO: 366 - protein, 11 aa, Homo sapiens
QKSVWHGSDPN

SEQ ID NO: 367 - protein, 19 aa, Homo sapiens
QVHAGHWEWIGGEPVAFTN

SEQ ID NO: 368 - protein, 18 aa, Homo sapiens
QLSRSHSSGSVLPLGELE

SEQ ID NO: 369 - protein, 19 aa, Homo sapiens
QESSWGKNVSSTESGRLFK

SEQ ID NO: 370 - protein, 13 aa, Homo sapiens
QRPEDAEDGAEGG

SEQ ID NO: 371 - protein, 12 aa, Homo sapiens
QHDGSGNDCEPV

SEQ ID NO: 372 - protein, 7 aa, Homo sapiens
QIECVCP

SEQ ID NO: 373 - protein, 12 aa, Homo sapiens
QSLDTPGASCGI

SEQ ID NO: 374 - protein, 12 aa, Homo sapiens
QLHILLEVRDHL

SEQ ID NO: 375 - skipped

SEQ ID NO: 376 - protein, 9 aa, Homo sapiens
QCKCRPNVT

SEQ ID NO: 377 - skipped

SEQ ID NO: 378 - protein, 18 aa, Homo sapiens
QRRLDFLAKHILHCKAKY

SEQ ID NO: 379 - protein, 10 aa, Homo sapiens
QKVEEVKPLE

SEQ ID NO: 380 - protein, 7 aa, Homo sapiens
QNPDAPE

SEQ ID NO: 381 - protein, 20 aa, Homo sapiens
QFEENWYNTYSSNIYKHGDT

SEQ ID NO: 382 - protein, 10 aa, Homo sapiens
QPVCFPPRPR

SEQ ID NO: 383 - protein, 16 aa, Homo sapiens
QDGKIVDDVWVVRPLF

SEQ ID NO: 384 - protein, 11 aa, Homo sapiens
QCYSGYALAED

SEQ ID NO: 385 - protein, 18 aa, Homo sapiens
QVEVNITEFPYTPSPPEH

SEQ ID NO: 386 - protein, 17 aa, Homo sapiens
QPTFTTRLDLDAAGAGA

SEQ ID NO: 387 - protein, 4 aa, Homo sapiens
QKCP

SEQ ID NO: 388 - protein, 11 aa, Homo sapiens
QVDSEEGLLST

SEQ ID NO: 389 - protein, 14 aa, Homo sapiens
QSSGENCDVVVNTL

SEQ ID NO: 390 - protein, 13 aa, Homo sapiens
QEESLFLISGKPE

SEQ ID NO: 391 - protein, 9 aa, Homo sapiens
QASVENGNA

SEQ ID NO: 392 - protein, 13 aa, Homo sapiens
QEESPSVISGKPE

SEQ ID NO: 393 - protein, 12 aa, Homo sapiens
QVSCPYDSMKHW

SEQ ID NO: 394 - protein, 18 aa, Homo sapiens
QERRKRKRSKRKKEVPFF

SEQ ID NO: 395 - protein, 17 aa, Homo sapiens
QRGGPCTNHEPPALEEG

SEQ ID NO: 396 - protein, 15 aa, Homo sapiens
QREGTREHLRERAKG

SEQ ID NO: 397 - protein, 4 aa, Homo sapiens
QGLV

SEQ ID NO: 398 - protein, 8 aa, Homo sapiens
QASPPRRL

SEQ ID NO: 399 - protein, 8 aa, Homo sapiens
QDEGILPS

SEQ ID NO: 400 - protein, 20 aa, Homo sapiens
QEVFLKTKDDAINGDKKAKK

SEQ ID NO: 401 - protein, 19 aa, Homo sapiens
QKVAPSSTMSTHPSRRRPN

SEQ ID NO: 402 - protein, 20 aa, Homo sapiens
QLEMEKNAEPTSKGTPRRKH

SEQ ID NO: 403 - protein, 20 aa, Homo sapiens
QRKPRPLDPGEGSKDTDSSA

SEQ ID NO: 404 - protein, 15 aa, Homo sapiens
QDSLFYSSNEFEEYP

SEQ ID NO: 405 - protein, 14 aa, Homo sapiens
QEEKNKDNIGFHHL

SEQ ID NO: 406 - skipped

SEQ ID NO: 407 - protein, 10 aa, Homo sapiens
QFNAFSYHFR

SEQ ID NO: 408 - protein, 12 aa, Homo sapiens
QMAVKKYLAAVL

SEQ ID NO: 409 - protein, 6 aa, Homo sapiens
QRVKNK

SEQ ID NO: 410 - protein, 7 aa, Homo sapiens
QVLETVA

SEQ ID NO: 411 - protein, 4 aa, Homo sapiens
QDAA

SEQ ID NO: 412 - protein, 5 aa, Homo sapiens
QVEIL

SEQ ID NO: 413 - protein, 17 aa, Homo sapiens
QEKREIGDEENSAKFPI

SEQ ID NO: 414 - protein, 6 aa, Homo sapiens
QATRDA

SEQ ID NO: 415 - protein, 19 aa, Homo sapiens
QRRPYRRRRFPPYYMRRPY

SEQ ID NO: 416 - protein, 5 aa, Homo sapiens
QPGFS

SEQ ID NO: 417 - protein, 12 aa, Homo sapiens
QLELPCPDPSTP

SEQ ID NO: 418 - protein, 10 aa, Homo sapiens
QVGLPILYFS

SEQ ID NO: 419 - protein, 6 aa, Homo sapiens
QSIVCT

SEQ ID NO: 420 - protein, 4 aa, Homo sapiens
QLAD

SEQ ID NO: 421 - protein, 4 aa, Homo sapiens
QVEA

SEQ ID NO: 422 - protein, 12 aa, Homo sapiens
QPTRRPRPGTGP

SEQ ID NO: 423 - protein, 4 aa, Homo sapiens
QPKK

SEQ ID NO: 424 - protein, 12 aa, Homo sapiens
QLSAKKYLESLM

SEQ ID NO: 425 - protein, 13 aa, Homo sapiens
QMAVKKYLNSILN

SEQ ID NO: 426 - protein, 5 aa, Homo sapiens
QKLPK

SEQ ID NO: 427 - protein, 14 aa, Homo sapiens
QGVVTVFKEAPRYP

SEQ ID NO: 428 - protein, 11 aa, Homo sapiens
QSPEPSPAPGP

SEQ ID NO: 429 - protein, 15 aa, Homo sapiens
QARSSSYSGEYGGGG

SEQ ID NO: 430 - protein, 9 aa, Homo sapiens
QDVPLAALE

SEQ ID NO: 431 - protein, 15 aa, Homo sapiens
QKDRGSGKAFCVDGE

SEQ ID NO: 432 - protein, 17 aa, Homo sapiens
QETLANSSIGRLCLAFL

SEQ ID NO: 433 - skipped

SEQ ID NO: 434 - protein, 6 aa, Homo sapiens
QAGGKP

SEQ ID NO: 435 - protein, 8 aa, Homo sapiens
QYGEPREG

SEQ ID NO: 436 - protein, 19 aa, Homo sapiens
QLADDGLVALDWVVGPCVR

SEQ ID NO: 437 - protein, 6 aa, Homo sapiens
QRLAAK

SEQ ID NO: 438 - protein, 8 aa, Homo sapiens
QSALIGKN

SEQ ID NO: 439 - protein, 10 aa, Homo sapiens
QAVSTAHPPT

SEQ ID NO: 440 - skipped

SEQ ID NO: 441 - protein, 16 aa, Homo sapiens
QNLAKAETKLHTSNSG

SEQ ID NO: 442 - skipped

SEQ ID NO: 443 - protein, 4 aa, Homo sapiens
QVYA

SEQ ID NO: 444 - protein, 6 aa, Homo sapiens
QGKTVI

SEQ ID NO: 445 - protein, 7 aa, Homo sapiens
QVTYVRS

SEQ ID NO: 446 - protein, 14 aa, Homo sapiens
QAAKEFIAWLVKGR

SEQ ID NO: 447 - protein, 8 aa, Homo sapiens
QPGSRRGG

SEQ ID NO: 448 - protein, 4 aa, Homo sapiens
QKLY

SEQ ID NO: 449 - protein, 12 aa, Homo sapiens
QISRSRFLLDSD

SEQ ID NO: 450 - skipped

SEQ ID NO: 451 - protein, 10 aa, Homo sapiens
QSCGPGRGPV

SEQ ID NO: 452 - protein, 10 aa, Homo sapiens
QGPPGPAGEE

SEQ ID NO: 453 - protein, 16 aa, Homo sapiens
QGPPGSRGPPGMRGAK

SEQ ID NO: 454 - protein, 7 aa, Homo sapiens
QGKAGAP

SEQ ID NO: 455 - protein, 13 aa, Homo sapiens
QGDDGDPGPMGPA

SEQ ID NO: 456 - protein, 5 aa, Homo sapiens
QLGPP

SEQ ID NO: 457 - protein, 10 aa, Homo sapiens
QGAPGPAGEE

SEQ ID NO: 458 - protein, 10 aa, Homo sapiens
QGPIGPPGEE

SEQ ID NO: 459 - protein, 10 aa, Homo sapiens
QGGKGDPGRP

SEQ ID NO: 460 - protein, 4 aa, Homo sapiens
QGSP

SEQ ID NO: 461 - protein, 10 aa, Homo sapiens
QASLYLLEAT

SEQ ID NO: 462 - protein, 7 aa, Homo sapiens
QGVKGHA

SEQ ID NO: 463 - protein, 8 aa, Homo sapiens
QIGNTGPK

SEQ ID NO: 464 - skipped

SEQ ID NO: 465 - protein, 4 aa, Homo sapiens
QPNG

SEQ ID NO: 466 - protein, 5 aa, Homo sapiens
QVLDT

SEQ ID NO: 467 - protein, 4 aa, Homo sapiens
QAAR

SEQ ID NO: 468 - skipped

SEQ ID NO: 469 - protein, 10 aa, Homo sapiens
QGPKGEKGAN

SEQ ID NO: 470 - protein, 12 aa, Homo sapiens
QPVYKPYLTLCA

SEQ ID NO: 471 - protein, 20 aa, Homo sapiens
QNTEYEDEKTLKNPKYKDRA

SEQ ID NO: 472 - protein, 9 aa, Homo sapiens
QRHLAGLAV

SEQ ID NO: 473 - protein, 8 aa, Homo sapiens
QECCSPEL

SEQ ID NO: 474 - protein, 18 aa, Homo sapiens
QPEPAPPRPSGPATAEDP

SEQ ID NO: 475 - protein, 15 aa, Homo sapiens
QCYNFGWYPESPVCE

SEQ ID NO: 476 - protein, 7 aa, Homo sapiens
QKWKYNR

(19) United States
(12) Patent Application Publication
(10) Pub. No.: US 2026/0226128 A1
(43) Pub. Date: Aug. 6, 2026

(54) Capped Peptides and Methods for Using the Same

(71) Applicant: The Board of Trustees of the Leland Stanford Junior University, Stanford, CA (US)

(72) Inventors: Jonathan Z. Long, Stanford, CA (US); Amanda L. Wiggenhorn, Stanford, CA (US)

(21) Appl. No.: 19/152,795

(22) PCT Filed: Feb. 8, 2024
(86) PCT No.: PCT/US2024/015002

§ 371 Date: Jul. 31, 2025

Related U.S. Application Data
(60) Provisional application No. 63/444,409, filed on Feb. 9, 2023.

(51) Int. Cl. C07K 14/575; A61K 38/00; A61P 3/04; C07K 14/47; C07K 14/475

(52) U.S. Cl. CPC C07K 14/575; A61P 3/04; C07K 14/4702; C07K 14/475; A61K 38/00; C07K 2319/90

(57) ABSTRACT

Capped peptides are provided. Aspects of the capped peptides include an N-terminal pyroglutamylation modification and a C-terminal amidation modification. In some instances, the capped peptides are not Thyrotropin-releasing hormone (TRH) or (Gonadotropin-releasing hormone (GnRH)/luteinizing hormone-releasing hormone (LHRH). Also provided are methods of administering capped peptides to a subject, e.g., to treat the subject for a condition, such as a disorder or disease condition, and pharmaceutical compositions that include the capped peptides.

### CROSS-REFERENCE

Pursuant to 35 U.S.C. § 119 (e), this application claims priority to the filing date of U.S. Provisional Patent Application No. 63/444,409, filed Feb. 9, 2023, which application is incorporated herein by reference in its entirety.

### INCORPORATION BY REFERENCE OF SEQUENCE LISTING XML FILE

A Sequence Listing is provided herewith as a Sequence Listing XML, “STAN-1907WO_S21-391_SEQ_LIST”, created on Feb. 8, 2024 and having a size of 389,698 bytes. The contents of the Sequence Listing XML are incorporated herein by reference in their entirety.

### INTRODUCTION

Peptide hormones and neuropeptides are fundamental signaling molecules that mediate cell-cell communication (Hook et al., J. Am. Soc. Mass Spectrom. 2018 29:807-816). These signaling molecules are produced by proteolytic cleavage of secreted preproproteins and released extracellularly via the classical secretory pathway. Once secreted, peptide hormones and neuropeptides act on cognate receptors to regulate nearly all aspects of homeostasis and physiology. Because of their potent and powerful physiologic actions, peptide hormones and neuropeptides have attracted considerable pharmaceutical interest as starting points for the development of therapeutics across for multiple human diseases (Sanyal et al., Lancet. 2018 392:2705-2717; Sikich et al., N. Engl. J. Med. 2021 385:1462-1473; Wilding et al., N. Engl. J. Med. 2021 384:989-1002).

The biosynthesis of signaling peptides often involves post-translational enzymatic modification of C- and/or N-termini. Approximately half of all peptide hormones and neuropeptides possess a C-terminal amide instead of a free carboxylate (e.g., GLP-1) (Eipper et al., Annu. Rev. Neurosci. 1992 15:57-85). This modification is installed by the action of PAM (peptidyl-glycine alpha-amidating monooxygenase), an enzyme that cleaves off a C-terminal glycine to leave an amide terminus (Czyzyk et al., Dev. Biol. 2005 287:301-313; Eipper et al., Annu. Rev. Neurosci. 1983 15:57-85). In addition, chemical modifications of the N-terminus include N-acetylation (Guo et al., Proc. Natl. Acad. Sci. 2004 101:11797-11802), N-formylation (Chen et al., Nat. Commun. 2022 13:1-13), and N-pyroglutamylation (Lindemans et al., Front. Endocrinol. 2010 2:1-6). Combinations of C- and N-terminal modifications can also be found within the same peptide (Lindemans et al., Front. Endocrinol. 2010 2:1-6). In the cases where they have been examined, C- and N-termini modifications can both increase peptide stability and also enhance receptor affinity (Van Coillie et al., Nucleic Acids Res. 1998 51: D523-D531; Goren and Bauce, Mol Pharmacol. 1977 13:606-614).

Historically, peptide hormones and neuropeptides-like molecules have been discovered via laborious and time-consuming activity-guided fractionation strategies (Harris and Lerner, Nature 1957 179:1346-1347; Schally et al., Science. 1971 173:1036-1038). More recently, shotgun mass spectrometry-based approaches using available mammalian genome information have been developed for the detection of peptide fragments from tissues and fluids (Fälth et al., Mol. Cell. Proteomics 2007 6:1188-1197; Gupta et al., J. Proteome Res. 2010 9:5065-5075; Tagore et al., Nat. Chem. Biol. 2009 5:23-25). However, the existing mass spectrometry methods typically identify few peptides (e.g., low coverage). In addition, the potential bioactivity and/or signaling of the peptides detected remains undefined.

### SUMMARY

The present inventors reasoned that knowledge of primary sequences indicative of post-translational processing of peptide termini, combined with a targeted mass spectrometry approach using authentic peptide standards, might enable de novo prediction and high-sensitivity detection of previously unknown peptide cleavage products. Importantly, these predicted cleavage products would harbor C- and N-terminal modifications that resemble that of other peptide hormones and neuropeptides, thereby suggesting also potential signaling activity. Using this strategy, the inventors report here the discovery of a large and sequence-diverse class of orphan blood-borne mammalian peptides called “capped peptides.” Capped peptides are defined by the co-incident presence of two post-translational modifications, N-terminal pyroglutamylation and C-terminal amidation, that function as terminal “caps” of the intervening peptide sequence. Capped peptides also exhibit regulatory characteristics similar to other signaling peptides, including dynamic changes to their circulating levels in response to physiologic and environmental state. In vitro and in vivo functional assays establish signaling and bioactivity for three orphan capped peptides: CAP-TAC1 is a tachykinin neuropeptide-like molecule that exhibits nanomolar agonist activity at multiple mammalian tachykinin receptors, CAP-GDF15, derived from the prepropeptide region of the anorexigenic hormone GDF15, is itself a novel 12-mer hypophagic peptide, and CAP-WNT9A is a growth hormone secretagogue peptide. The studies reported herein demonstrate that N- and C-terminal capping is present in a much larger number of endogenous secreted peptides than previously anticipated. Capped peptides therefore constitute a class of secreted signaling peptides with the potential to broadly regulate cell-cell communication in mammalian physiology.

Capped peptides are provided. Aspects of the capped peptides include an N-terminal pyroglutamylation modification and a C-terminal amidation modification. In some instances, the capped peptides are not Thyrotropin-releasing hormone (TRH) or (Gonadotropin-releasing hormone (GnRH)/luteinizing hormone-releasing hormone (LHRH). Also provided are methods of administering capped peptides to a subject, e.g., to treat the subject for a condition, such as a disease or disorder condition, and pharmaceutical compositions that include the capped peptides.

### BRIEF DESCRIPTION OF THE FIGURES

FIG. 1. Genomic prediction and mass spectrometry detection of capped peptides in mouse plasma. (A) Schematic representation of capped peptide production from secreted, full-length preproprecursor proteins. Abbreviations are defined as: “N-term” is N-terminal; “C-term” is C-terminal; “SP” is signal peptide; “Gin” is glutamine; “pGlu” is pyroglutamyl. (B) Number of predicted mouse capped peptides and (C) the targeted mass spectrometry strategy for their detection. (D) Chemical structure of CAP-TAC1 (pGlu-FFGLM-NH2). (E) Representative extracted ion chromatogram at m/z=724.3 and (F) mirror fragmentation spectra of authentic CAP-TAC1 standard (pink) and the endogenous mouse plasma peak (blue). (G) Pie chart showing the number of detectable capped peptides in mouse plasma and (H) their quantitation. For (H), data are shown as means #SEM (N=5 mice).

FIG. 2. Sequence and gene-level analysis of capped peptides. (A) Pie chart and (B) representative examples of the distribution of mouse capped peptides based on their full-length preproprotein precursors. “SP” is signal peptide. (C) H-clustered heat map of mRNA expression for capped peptide preproprecursor home genes across mouse tissues and cell types. Data was obtained from BioGPS and shown as Z-score of the log-transformed value. (D) Frequency of each amino acid in capped peptides versus a Uniprot reference set of known peptide hormones and neuropeptides. (E) Heat map of amino acid frequency within four residues upstream and downstream of the N-terminal pyroglutamylation or (F) C-terminal amidation.

FIG. 3. Dynamic regulation of circulating capped peptide levels by different environmental and physiologic perturbations. (A) Heat map of the relative fold change for the indicated capped peptide in the indicated condition. (B-F) Representative peptide quantification for the indicated capped peptide in the indicated comparison. For (A-F), “Fasting/Fed” indicates a comparison between plasma from 3-month old male mice on chow diet versus age- and sex-matched controls after fasting for 16 h (N=3/group); “HFD/Chow” indicates a comparison between plasma from 4-month old male mice on chow diet versus 4-month old male mice after being on high fat diet (HFD, 60% kcal from fat) for 9 weeks (N=3/group); “LPS” indicates a comparison between plasma from 2-month old male mice following treatment with vehicle versus LPS (0.5 mg/kg, IP, 5 h, N=5/group); “6 AM/6 PM” indicates a comparison between plasma from 2-month old male mice at the beginning of the night cycle (6 AM) versus age- and sex-matched mice at the beginning of the day cycle (6 PM) (N=5/group); “Old/Young” indicates a comparison between plasma from 4-month old male mice versus 23-month old male mice (N=2/group); “Run/Sed” indicates a comparison between plasma from sedentary 3-month old male mice versus age- and sex-matched controls after an acute exhaustive 60 min run on a treadmill. For (B-F), data are shown as means±SEM. * P<0.05, ** P<0.01 versus control by Student's t-test.

FIG. 4. CAP-TAC1 is a potent tachykinin receptor agonist. (A) Schematic and annotation of the primary amino acid sequence for full-length mouse TAC1 preproprotein and its cleavage products. (B) Chemiluminescent signal intensity in PathHunter beta-arrestin CHO-K1 cells transfected with human TACR1 following treatment with the indicated concentration of CAP-TAC1 or substance P. (C) Relative levels of CAP-TAC1 or substance P in mouse plasma following incubation at 37° C. for the indicated time. For (B), N=2 per concentration; for (C), N=3 per condition. For (B,C), data are shown as means±SEM. * P<0.05, ** P<0.01 versus control by Student's t-test.

FIG. 5. Functional studies of CAP-GDF15 in feeding and energy balance in mice. (A) Schematic of the primary amino acid sequence for full-length mouse GDF15 preproprotein and annotation of CAP-GDF15 sequence as well as the canonical GDF15 hormone sequence. (B) Representative extracted ion chromatogram and (C) mirror tandem fragmentation spectra of CAP-GDF15 (pGlu-LELRLRVAAGR-NH2; +2 ion m/z=682.4) and the endogenous plasma peak. (D) Food intake, (E) respiratory exchange ratio (RER), and (F) ambulatory activity of 12-16-week diet-induced obese male mice following a single treatment of CAP-GDF15 (50 mg/kg, intraperitoneal) or vehicle control. (G) Food intake, (H) RER, and (I) ambulatory activity of 12-16-week diet-induced obese male mice following a single treatment of scrambled CAP-GDF15 (50 mg/kg, intraperitoneal, scrambled sequence: pGlu-GLEALRARLRV-NH2) or vehicle control. (J) 3-day food intake and (K) body weight of 12-16-week diet-induced obese male mice following treatment with CAP-GDF15 or scrambled CAP-GDF15 (50 mg/kg/day, IP), or vehicle control. Data are shown as means #SEM. For (D-F), N=12/group; for (G-I), N=8/group; for (J,K), N=7/group. For (D-1), injection occurred at time T=0 (5:00 μm) and data was collected for the following 16 hrs. For (D-K), ** P<0.01, *** P<0.001 by two-way ANOVA.

FIG. 6. Additional measurements from metabolic chambers of mice treated with CAP-GDF15. (A) VO2 and (B) VCO2 of 12-16-week diet-induced obese male mice following a single treatment of CAP-GDF15 (50 mg/kg, intraperitoneal) or vehicle control. (C) VO2 and (D) VCO2 of 12-16-week diet-induced obese male mice following a single treatment of scrambled CAP-GDF15 (50 mg/kg, intraperitoneal, scrambled sequence: pQGLEALRARLRV-NH2) or vehicle control. Data are shown as means±SEM. For (A-B), N=12/group; for (C-D), N=8/group. For (A-D), injection occurred at time T=0 (5:00 μm) and data was collected for the subsequent 16 hrs.

FIG. 7. Detection of human capped peptides and sequence alignment comparison to mouse. (A) Pie graph of detectable human capped peptides and diagram of the overlap between mouse-specific and human-specific capped peptides. (B) Number of human or mouse capped peptides detected in human or mouse plasma, stratified by those capped peptide with 100% conservation (top) or those that are not 100% conserved (human-specific, middle; mouse-specific, bottom). (C,D) Representative extracted ion chromatograms of the indicated capped peptides in plasma from the indicated species. (E) Phylogenetic alignment of all detectable capped peptides in human and mouse, with primary capped peptide sequences for select subclusters A-D shown on the right.

FIG. 8. Prediction, detection, and composition analysis of human capped peptides. (A) Schematic of numbers of predicted human capped peptides. (B) Quantification of detectable human capped peptide concentrations in human plasma (N=3). Data is shown as means±SEM, N=3. (C) Comparison of frequency of each amino acid between human capped peptides and known peptide hormones.

FIG. 9. Tissue distribution of mRNAs for home genes corresponding to human capped peptides. H-clustered heat map of mRNA expression for capped peptide preproprecursor home genes across human tissues and cell types, using GTEx as the reference database.

FIG. 10. CAP-TAC1 induces body weight loss independent of food intake. (A) Body weight change and (B) food intake of diet induced obese mice injected with vehicle (18:1:1 Saline: DMSO: Kolliphor EL), 10 mg/kg CAP-TAC1 (pGlu-FFGLM-NH2), and vehicle pair fed to CAP-TAC1 group. (N=6 for vehicle, N=7 for CAP-TAC1 and pair fed groups, error bars are SEM, and P values calculated with two-way ANOVA.)

FIG. 11. CAP-TAC1 acutely induces oxygen consumption over 24 hours. Oxygen consumption (VO2) of diet induced obese mice injected with a single injection at T=0 vehicle (18:1:1 Saline: DMSO: Kolliphor EL) or 10 mg/kg CAP-TAC1 (pGlu-FFGLM-NH2). (N=4 for vehicle and CAP-TAC1 groups, error bars are SEM, and P values calculated with two-way ANOVA.)

FIG. 12. Human CAP-GDF15 homologue inhibits food intake. Food intake of diet induced obese mice 2 hours post injection with vehicle or 50 mg/kg CAP-GDF15 human homologue (pGlu-LELHLRPQAAR-NH2). (N=8 mice/group, error bars are SEM, and P values calculated with two-sided Student's T test.)

FIG. 13. Functional screen in GH3 cells identifies CAP-WNT9A as a growth hormone secretagogue in vitro. (A) Z-score of media growth hormone concentration with 1 hour treatment of 10 μM of 100% conserved capped peptides growth hormone secretagogue activity in GH3 cells (N=1 for all peptides and N=3 for DMSO). (B) Media growth hormone concentration with 1 hour treatment of 10 μM CAP-WNT9A or DMSO (N=3/group). (Error bars are SEM.)

FIG. 14. Mouse capped peptides. (A) Predicted mouse capped peptides and (B-E) detected mouse capped peptides. (B-C) shows peptide information, (D) shows ion count integrations, and (E) shows concentrations in nM for detected mouse capped peptides.

FIG. 15. Human capped peptides. (A) Predicted human capped peptides and (B-E) detected human capped peptides. (B-C) shows peptide information, (D) shows ion count integrations, and (E) shows concentrations in nM for detected human capped peptides.

The invention is best understood from the following detailed description when read in conjunction with the accompanying drawings.

### Definitions

Before describing exemplary embodiments in greater detail, the following definitions are set forth to illustrate and define the meaning and scope of the terms used in the description.

Unless defined otherwise, all technical and scientific terms used herein have the same meaning as commonly understood by one of ordinary skill in the art to which this invention belongs. Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It must be noted that as used herein and in the appended claims, the singular forms “a”, “an”, and “the” include plural referents unless the context clearly dictates otherwise. For example, the term “an agent” refers to one or more agents, i.e., a single agent and multiple agents. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

A “pharmaceutical composition” refers to a formulation of a compound and a medium generally accepted in the art for the delivery of the biologically active compound to a mammal, e.g., humans. Such a medium can include a pharmaceutically acceptable delivery vehicle, carrier, diluent, or excipient.

While it may be possible for the compounds of the subject invention to be administered as the raw chemical, it is also possible to present them as a pharmaceutical composition. Accordingly, provided herein are pharmaceutical compositions which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. Pharmaceutically acceptable delivery vehicles and other therapeutic ingredients may include one or more excipients and/or one or more vehicles. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The term “peptide” used herein, refers to a polymeric form of amino acids of any length, which can include coded and non-coded amino acids, chemically or biochemically modified or derivatized amino acids, and polypeptides having modified peptide backbones.

The terms “subject,” “individual,” and “patient” are used interchangeably herein to refer to a mammal being assessed for treatment and/or being treated. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc.

The term “sample” with reference to a patient encompasses blood and other liquid samples of biological origin, solid tissue samples such as a biopsy specimen or tissue cultures or cells derived therefrom and the progeny thereof. The term also encompasses samples that have been manipulated in any way after their procurement, such as by treatment with reagents; washed; or enrichment for certain cell populations, such as diseased cells. The definition also includes samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's diseased cell, e.g., a sample comprising polynucleotides and/or polypeptides that is obtained from a patient's diseased cell (e.g., a cell lysate or other cell extract comprising polynucleotides and/or polypeptides); and a sample comprising diseased cells from a patient. A biological sample comprising a diseased cell from a patient can also include non-diseased cells.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome. In one example, a physician may attempt to predict the likelihood that a patient will survive.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of fatty liver disease in a mammal, particularly in a human, and includes: (a) inhibiting the disease, i.e., arresting its development; and (b) relieving the disease or its symptoms, i.e., causing regression of the disease or its symptoms.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of engineered cells to prevent or delay, to alleviate, or to arrest or inhibit development of the symptoms or conditions associated with disease or other diseases. The term “therapeutic effect” refers to the reduction, elimination, or prevention of the disease, symptoms of the disease, or side effects of the disease in the subject.

As used herein, a “therapeutically effective amount” refers to that amount of the therapeutic agent sufficient to treat or manage a disease or disorder. A therapeutically effective amount may refer to the amount of therapeutic agent sufficient to delay or minimize the onset of disease. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

“In combination with”, “combination therapy” and “combination products” refer, in certain embodiments, to the concurrent administration to a patient of the engineered proteins and cells described herein in combination with additional therapies, e.g., surgery, radiation, chemotherapy, and the like. When administered in combination, each component can be administered at the same time or sequentially in any order at different points in time. Thus, each component can be administered separately but sufficiently closely in time so as to provide the desired therapeutic effect.

“Concomitant administration” means administration of one or more components, such as engineered proteins and cells, known therapeutic agents, etc. at such time that the combination will have a therapeutic effect. Such concomitant administration may involve concurrent (i.e., at the same time), prior, or subsequent administration of components. A person of ordinary skill in the art would have no difficulty determining the appropriate timing, sequence and dosages of administration.

The term “sustained-release”, as in a sustained-release form, sustained-release composition or sustained-release formulation, is intended to include a form of an active ingredient, or formulation for an active ingredient, which has an extended in vivo half-life or duration of action. A sustained-release form may result from modification of the active ingredient, such as modifications that extend circulation residence time, decrease rates of degradation, decrease rates of clearance or the like, or may result from formulations or compositions which provide for extended release of the active ingredient, such as use of various liposomes, emulsions, micelles, matrices and the like. A controlled-release form or formulation is a type of sustained-release form or formulation.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### DETAILED DESCRIPTION

Capped peptides are provided. Aspects of the capped peptides include an N-terminal pyroglutamylation modification and a C-terminal amidation modification. In some instances, the capped peptides are not Thyrotropin-releasing hormone (TRH) or (Gonadotropin-releasing hormone (GnRH)/luteinizing hormone-releasing hormone (LHRH). In some instances, the capped peptides exhibit physiological activity, including, but not limited to, mammalian tachykinin receptor agonist activity, activating metabolic activity, hypophagic activity and growth hormone secretagogue activity. Also provided are methods of administering capped peptides to a subject in need thereof, e.g., to treat the subject for a condition, such as a disorder or disease condition, as well as pharmaceutical compositions that include capped peptides.

### Capped Peptides

As reviewed above, capped peptides are provided. Capped peptides of embodiments of the invention include an N-terminal pyroglutamylation modification and a C-terminal amidation modification, wherein the capped peptide is not Thyrotropin-releasing hormone (TRH) or (Gonadotropin-releasing hormone (GnRH)/luteinizing hormone-releasing hormone (LHRH).

Capped peptides are modified fragments of proteins. By “capped peptide” it is meant that the N-terminus and C-terminus of the peptide are modified. These modifications include chemical modifications. Types of modifications include N-terminal pyroglutamylation modification and C-terminal amidation modification. In an N-terminal pyroglutamylation modification, an N-terminal glutamine is modified to an N-terminal pyroglutamate. The pyroglutamylation reaction may be carried out enzymatically or chemically. In a C-terminal amidation modification, a C-terminal amino acid is modified to a C-terminal amide. The amidation reaction may be carried out enzymatically or chemically. In some cases, the amidation reaction may be carried out by the enzyme peptidyl-glycine alpha-amidating monooxygenase (PAM).

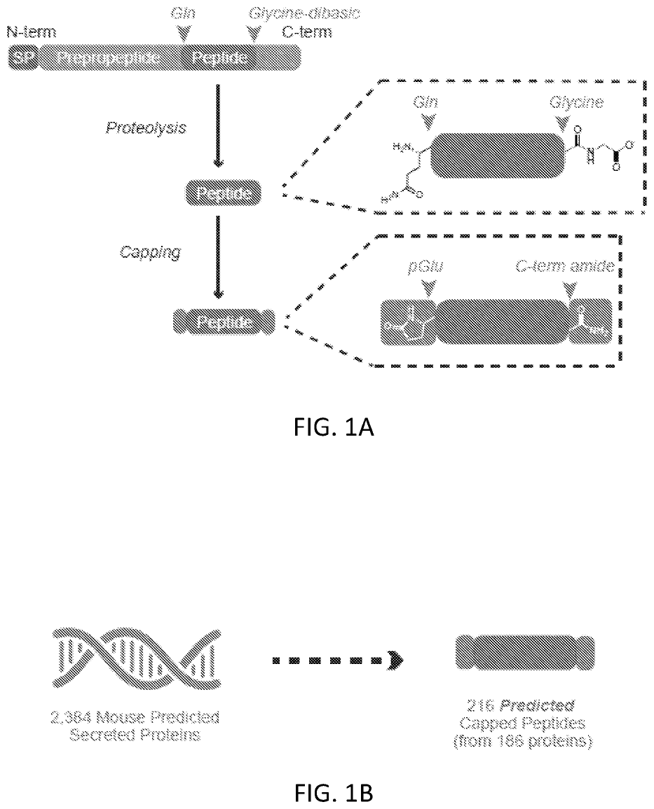

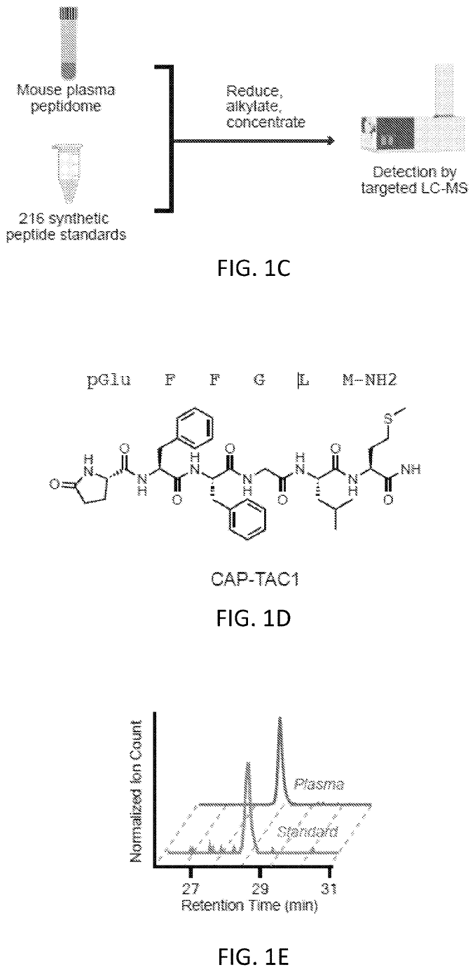

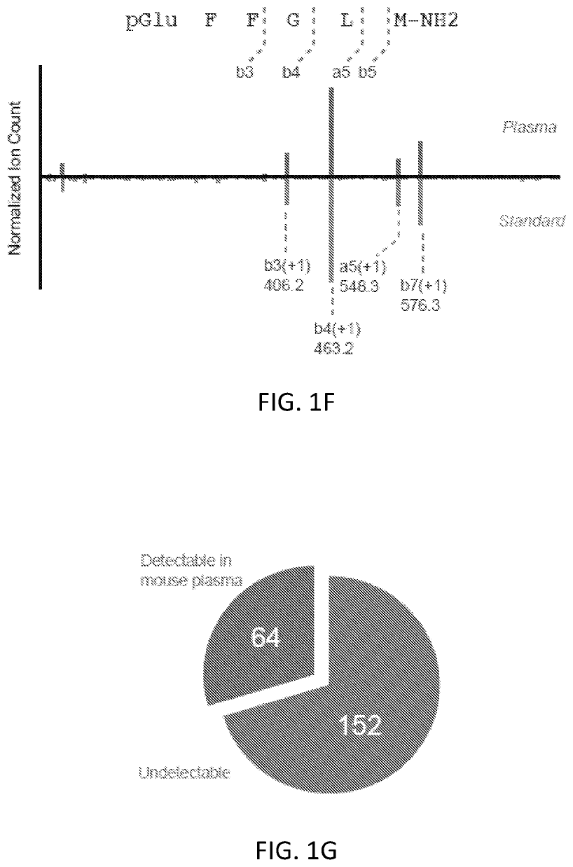

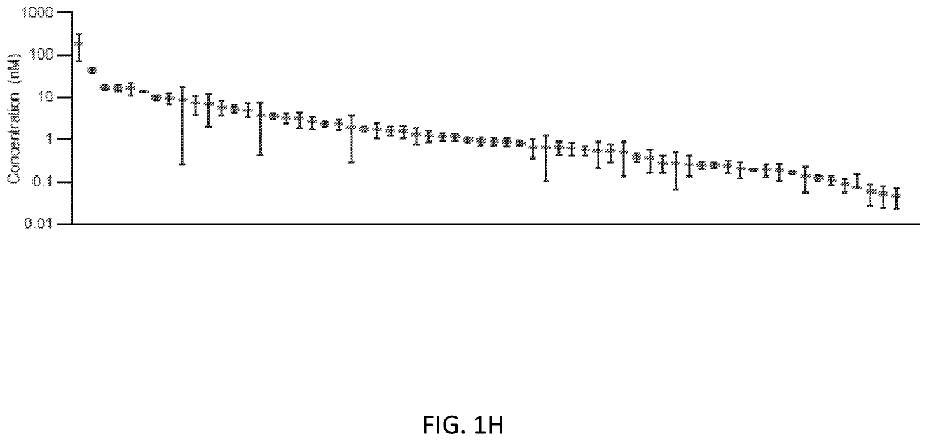

Capped peptides may be naturally occurring peptides or synthetic peptides. Peptides may be capped via post-translational modification or modified synthetically. Capped peptides may be produced from secreted proteins or synthesized proteins. Secreted proteins may be preproprecursor proteins. By “preproprecursor protein” it is meant that the protein is a protein precursor tagged with an N-terminal signal peptide that targets the protein for secretion. To produce a capped peptide from a secreted protein, the secreted protein is proteolyzed to produce a peptide, and then the peptide is post-translationally modified to produce a capped peptide. For example, an N-terminal glutamine amino acid can be post-translationally modified to an N-terminal pyroglutamyl amino acid and a C-terminal amino acid can be post translationally modified to a C-terminal amide (FIG. 1).

Capped peptides may be chemically synthesized and/or modified. For example, methods of chemically synthesizing capped peptides may include, but are not limited to, solid phase peptide synthesis and/or chemoselective ligation.

In some embodiments, the capped peptides may vary in size, ranging in some instances from 2 to 30 amino acid residues, such as 3 to 25 amino acid residues, and including 3 to 20 amino acid residues. Capped peptides may include coded and non-coded amino acids. Capped peptides may include, but are not limited to, chemically or biochemically modified amino acids, derivatized amino acids, and peptides having modified peptide backbones.

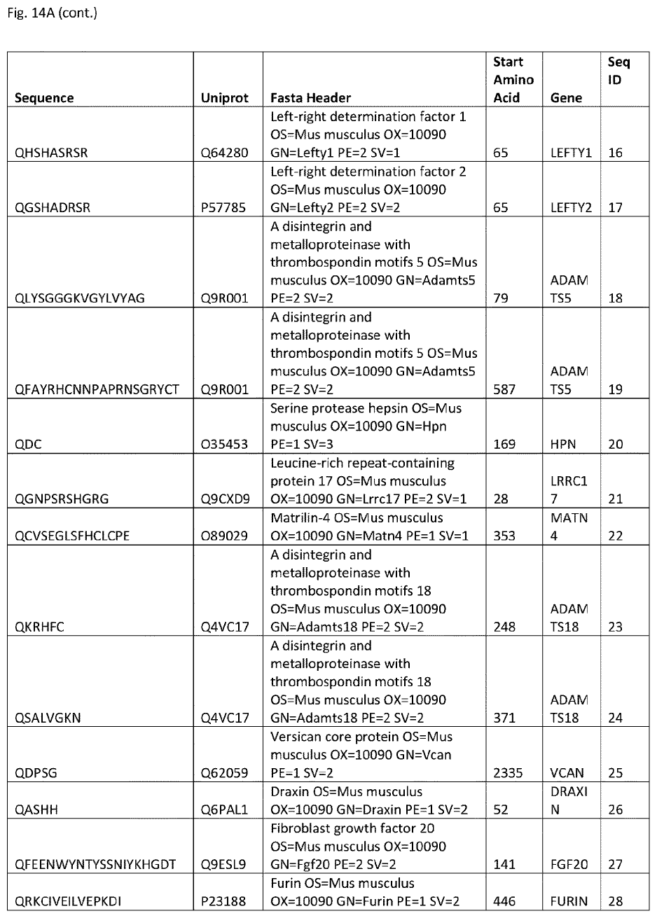

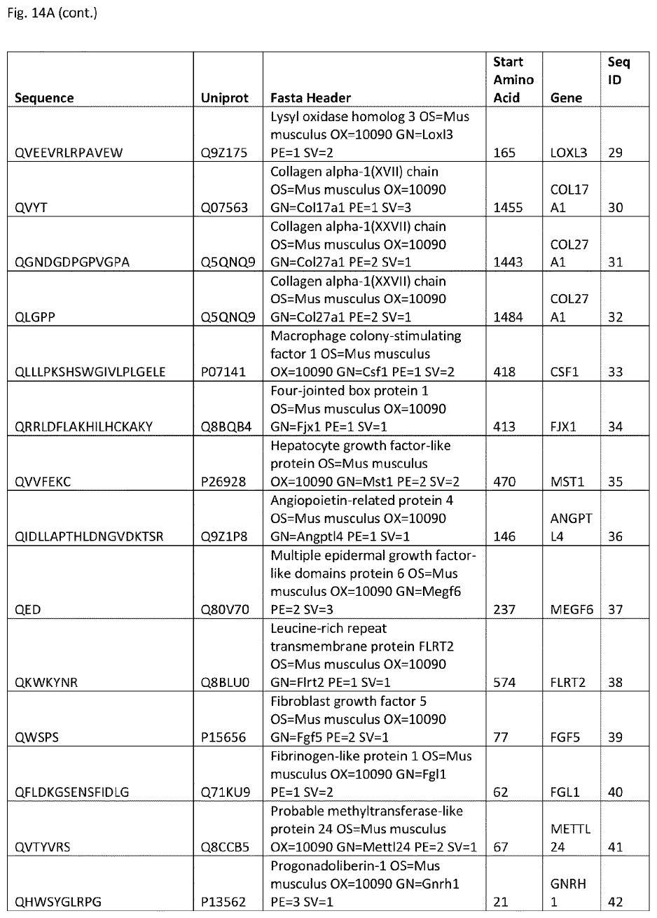

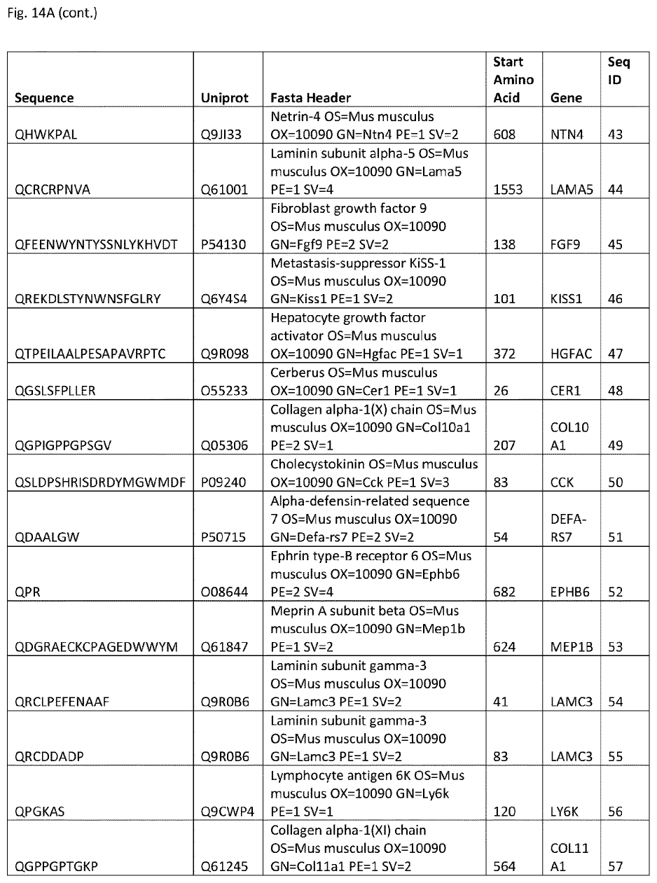

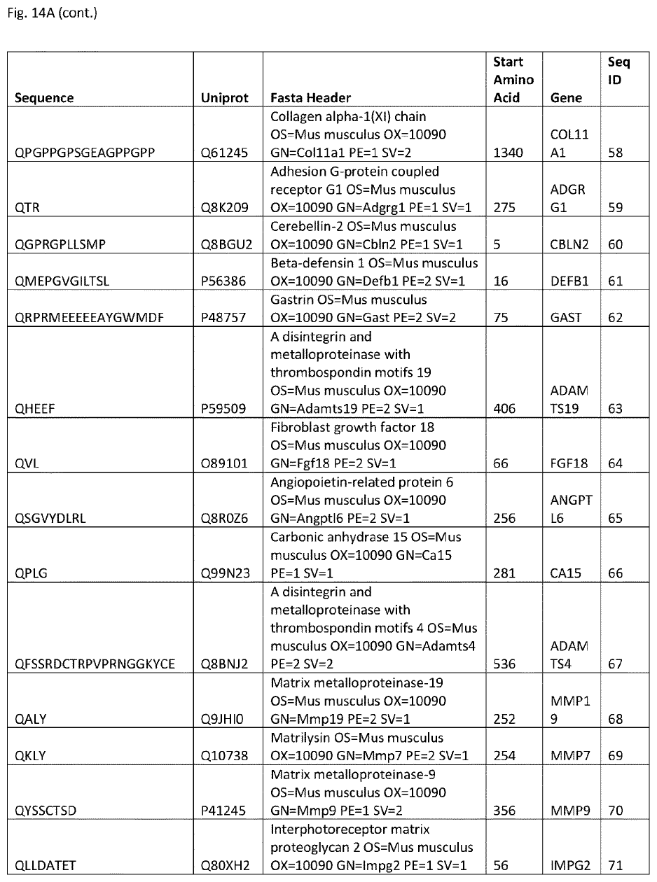

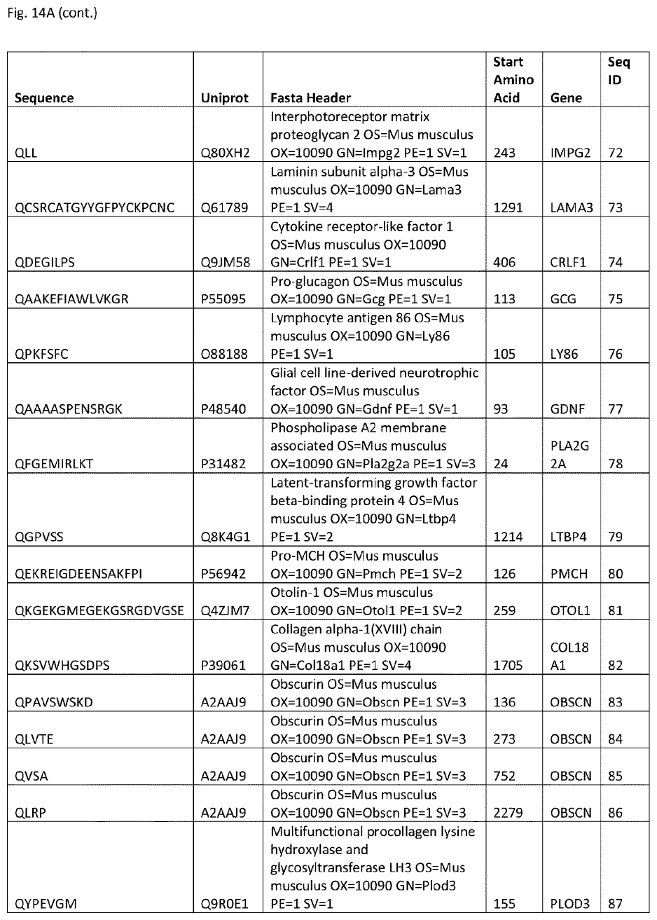

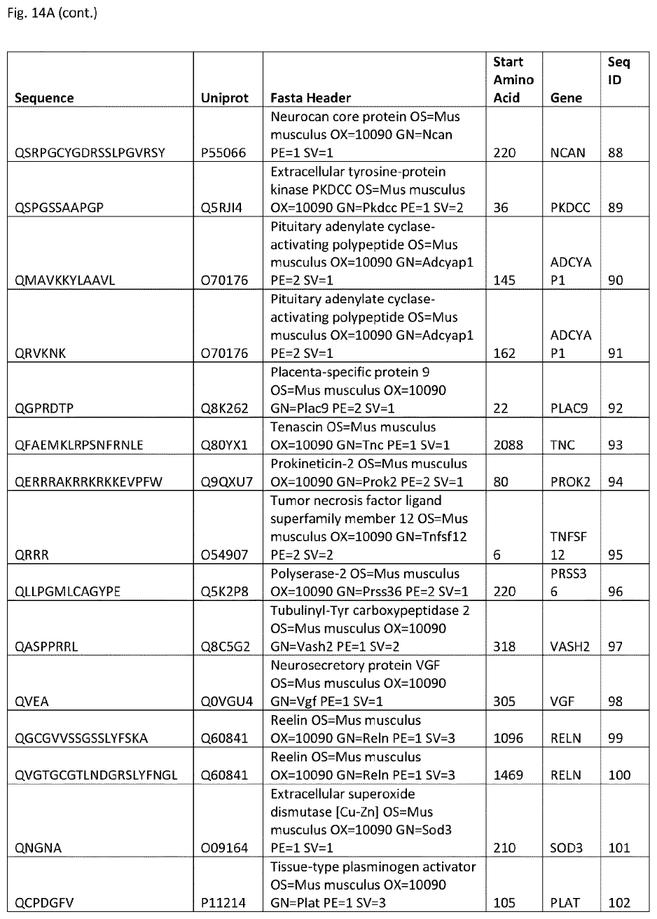

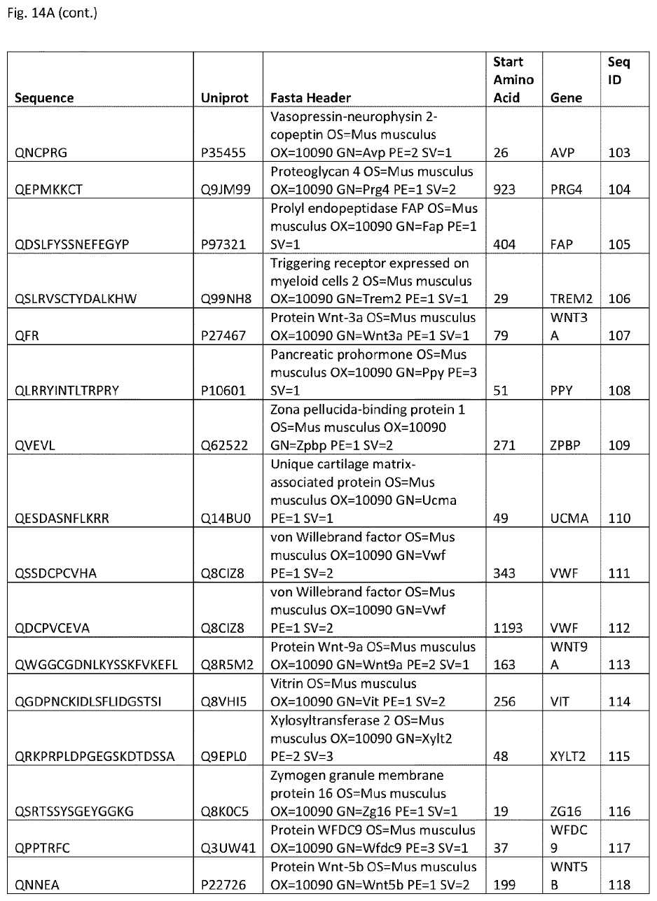

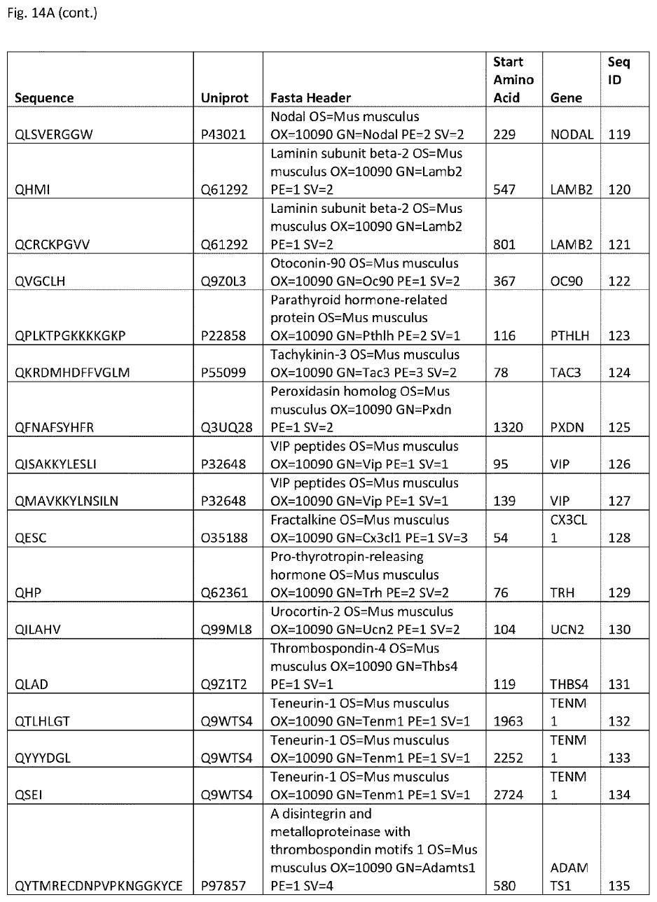

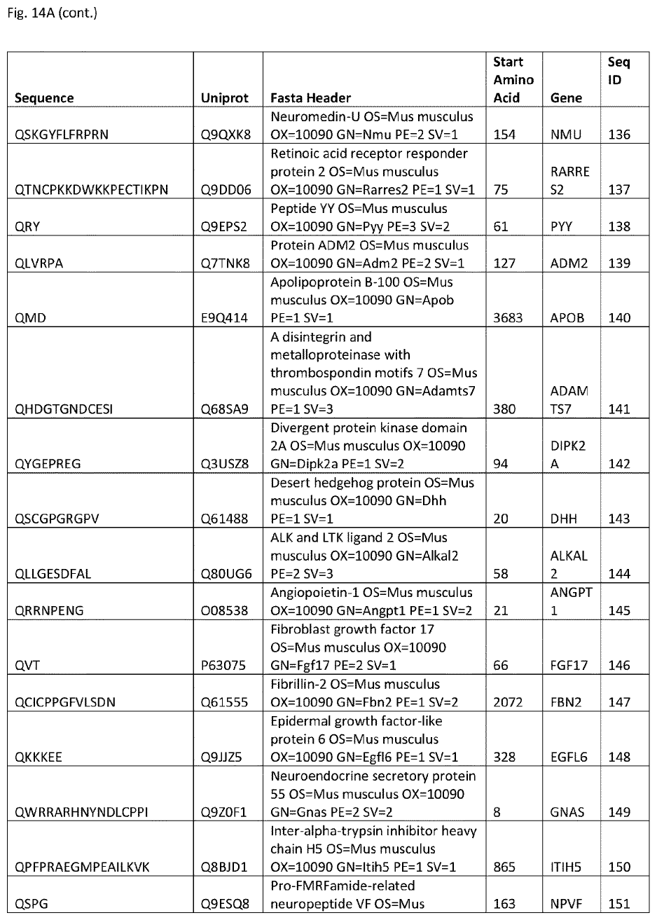

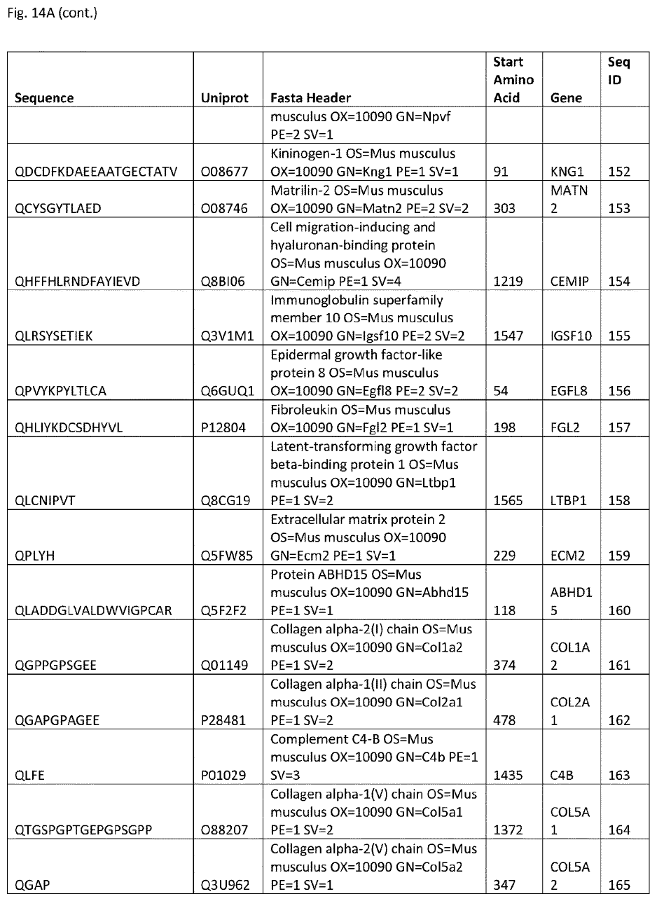

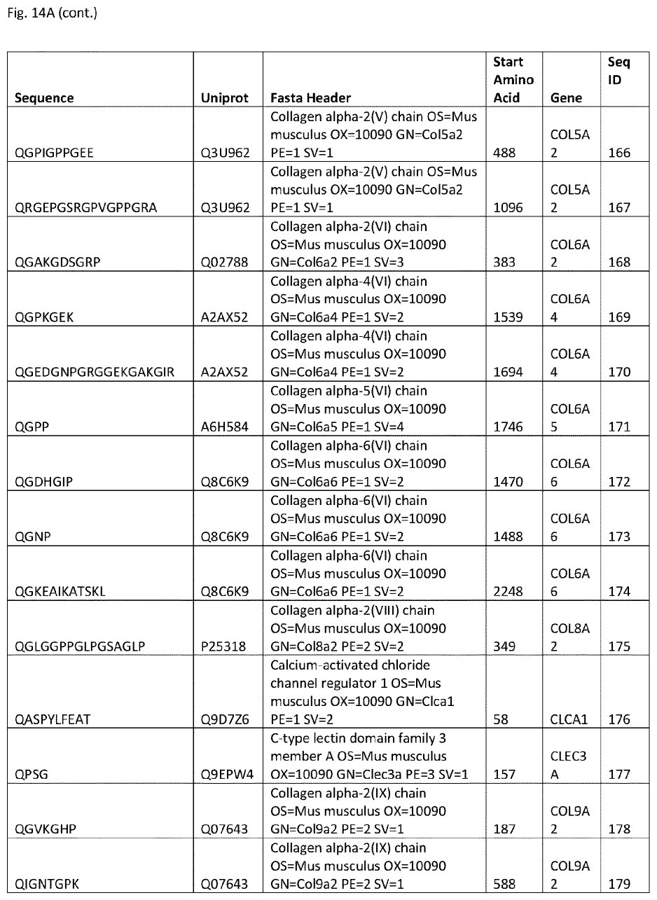

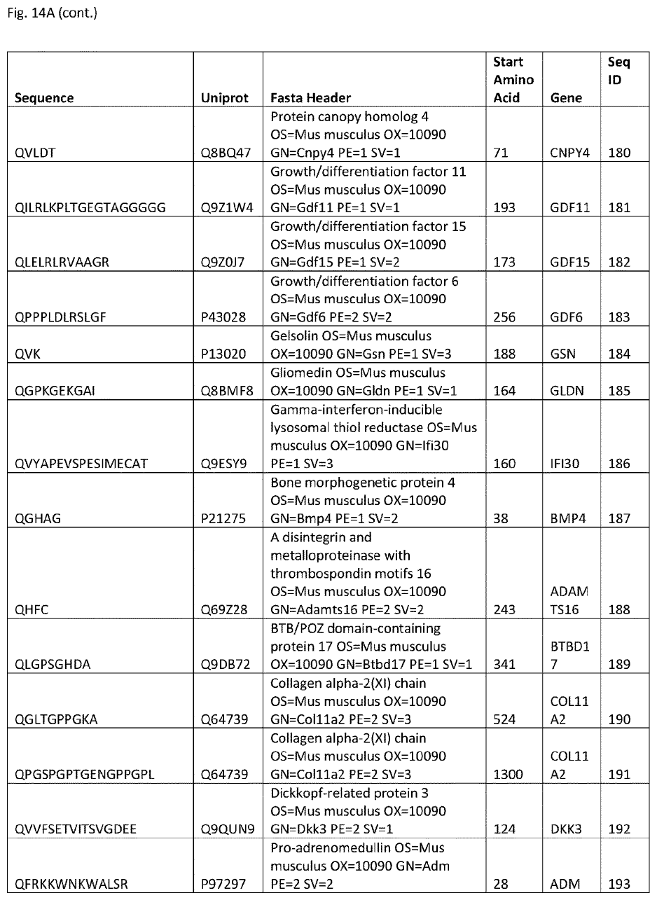

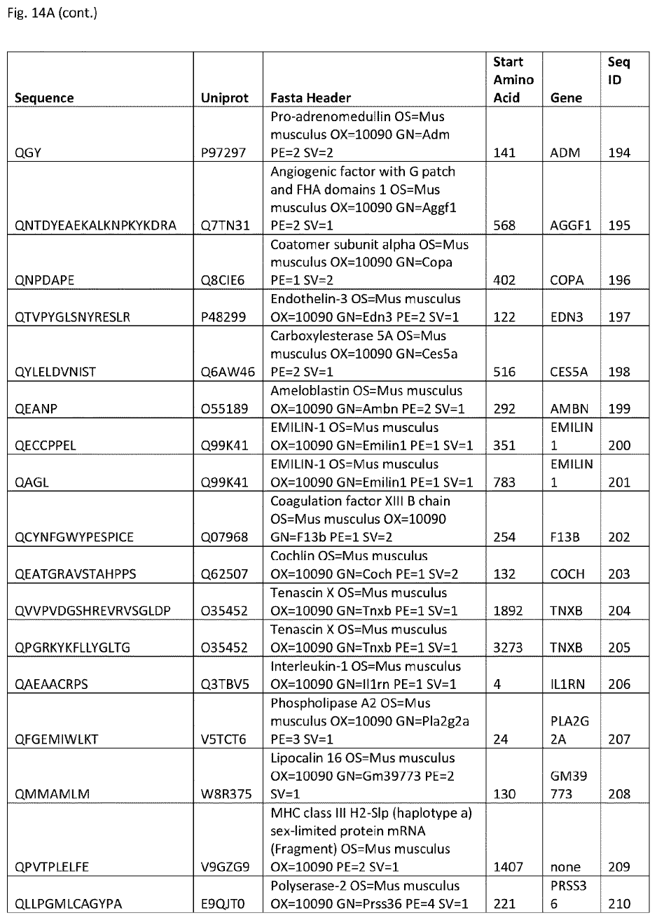

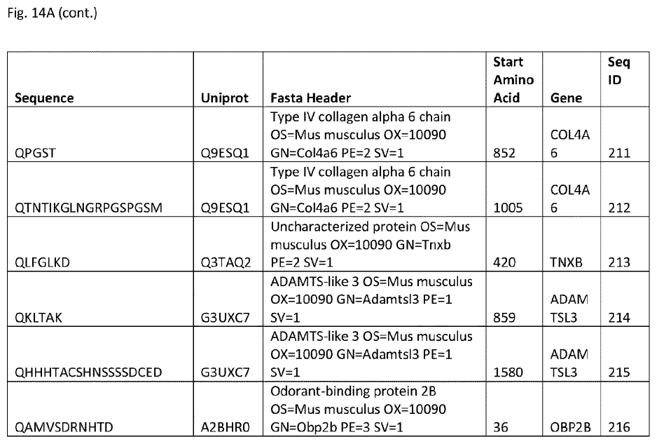

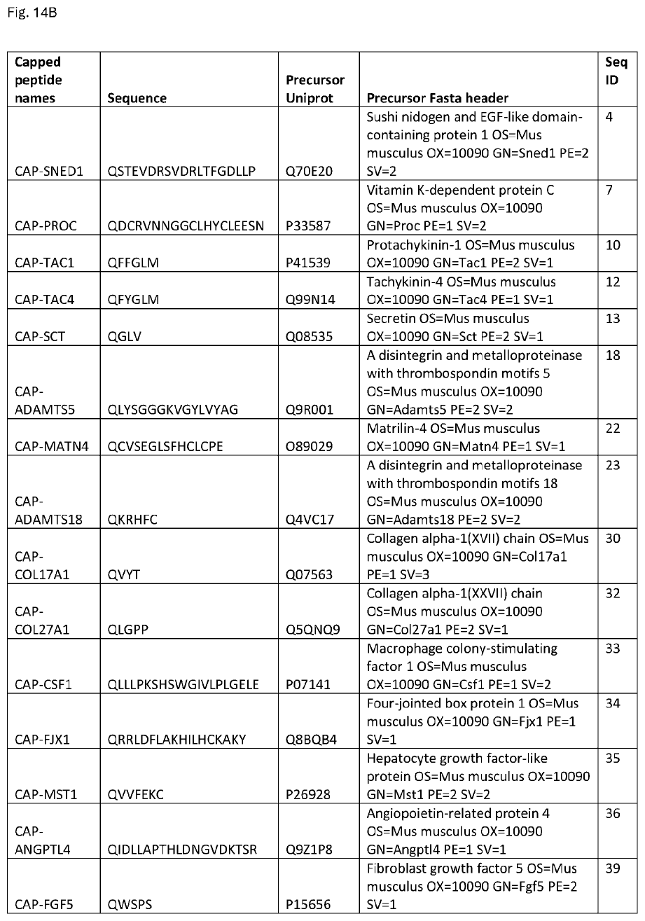

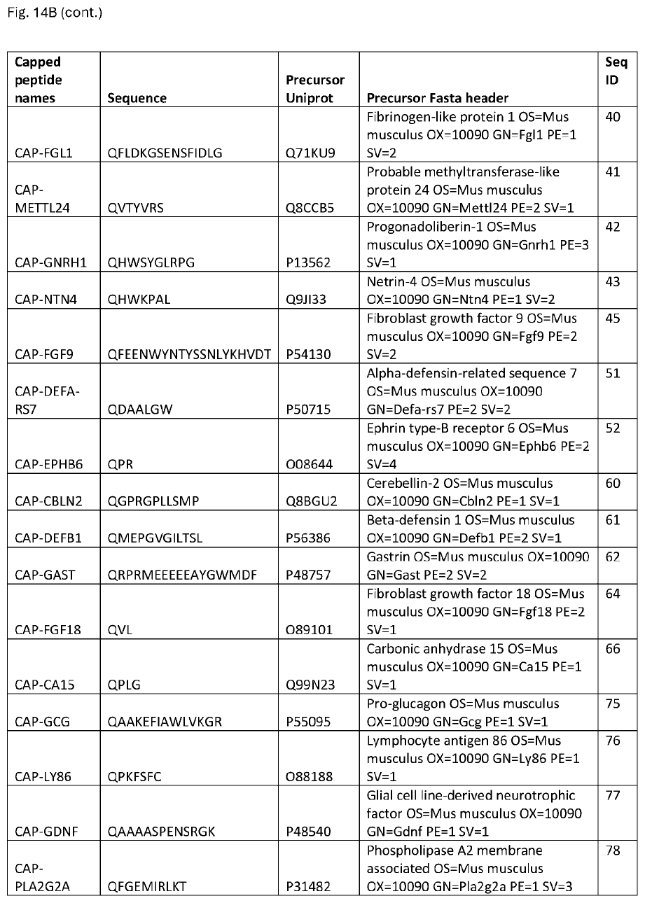

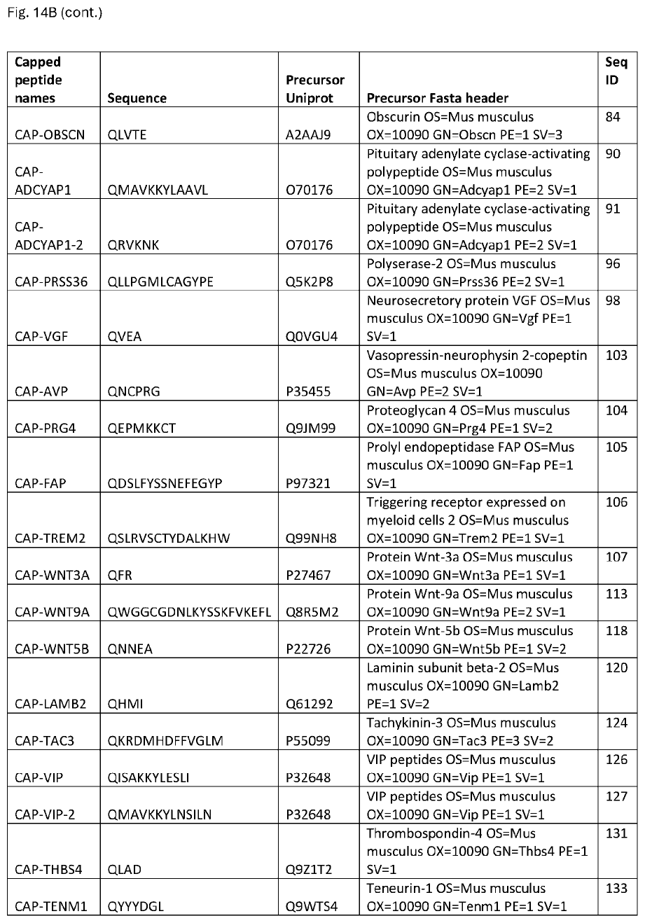

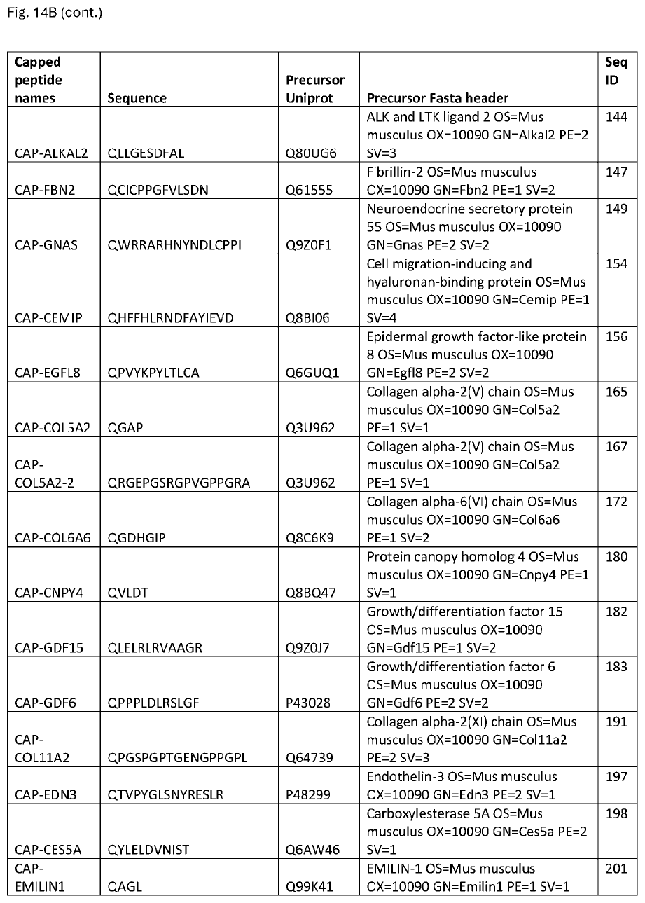

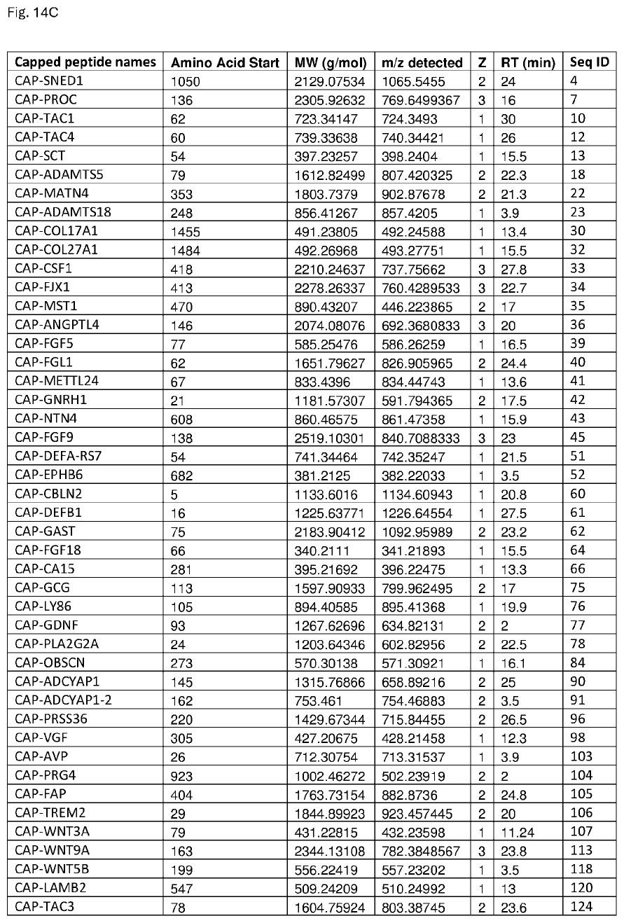

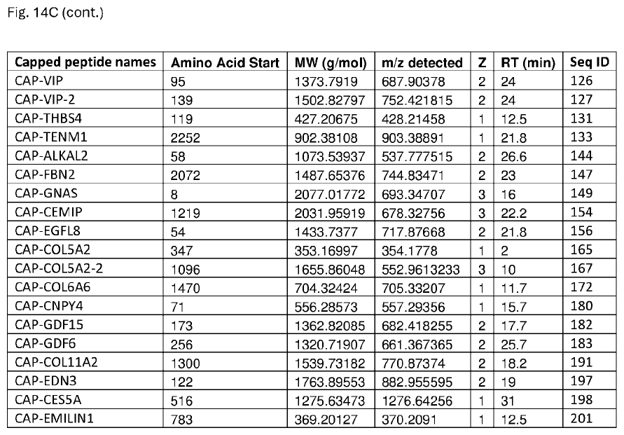

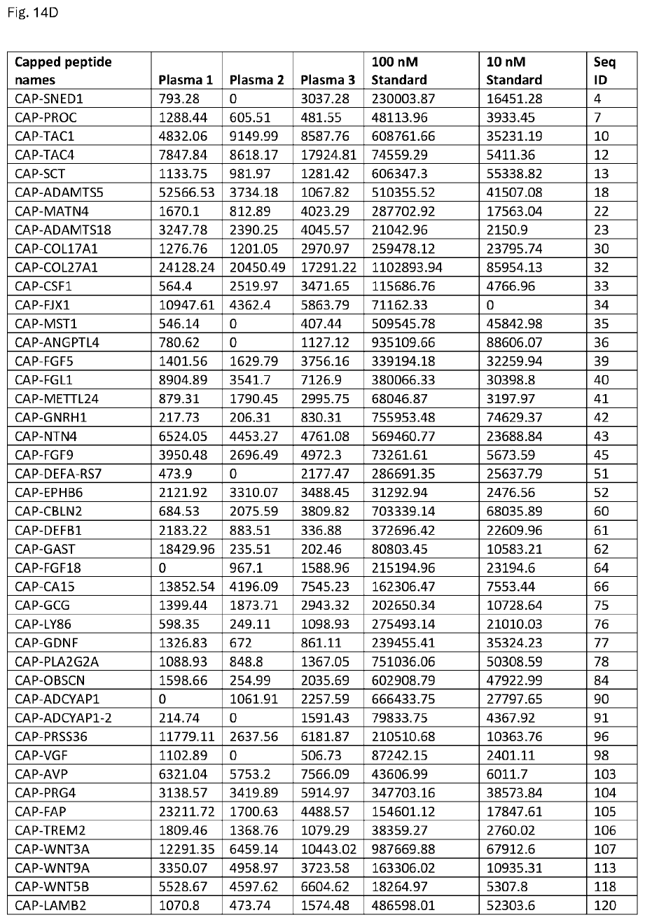

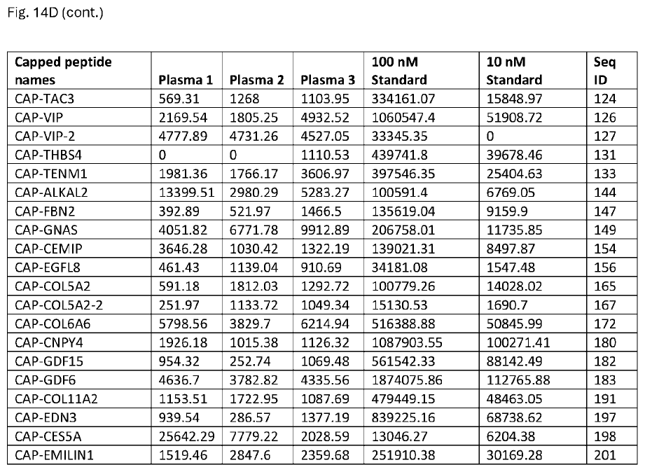

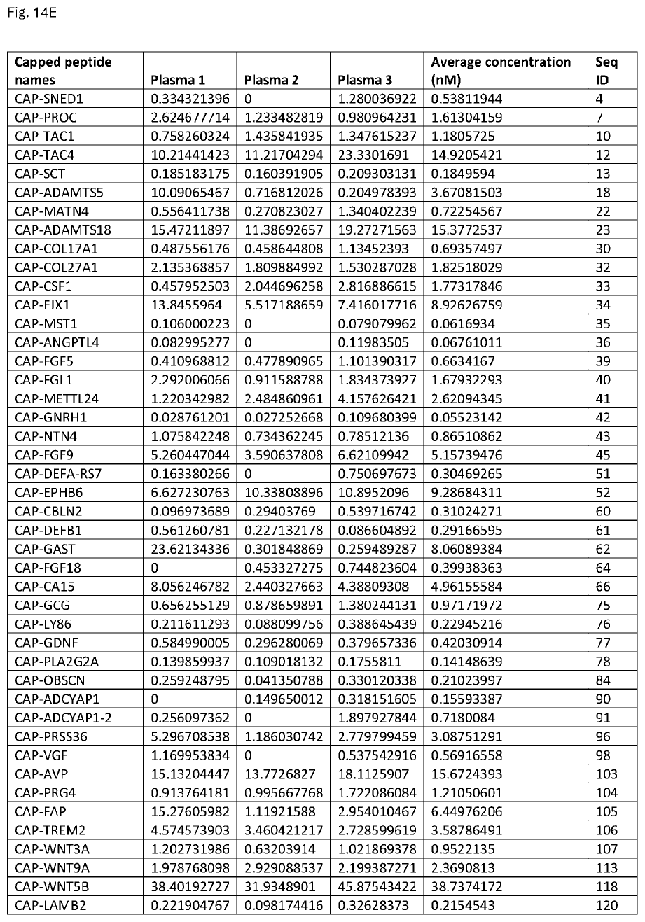

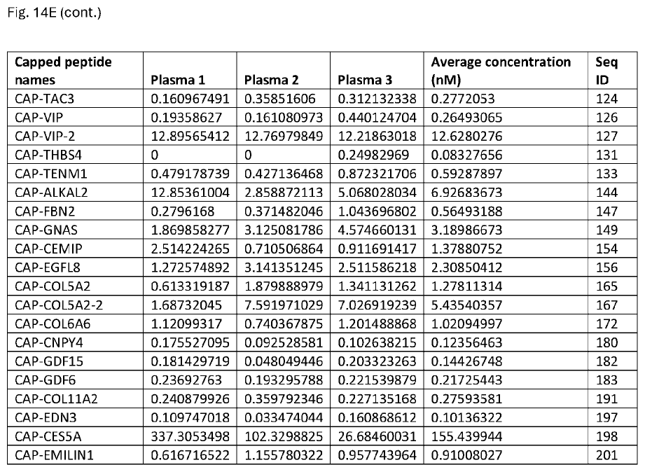

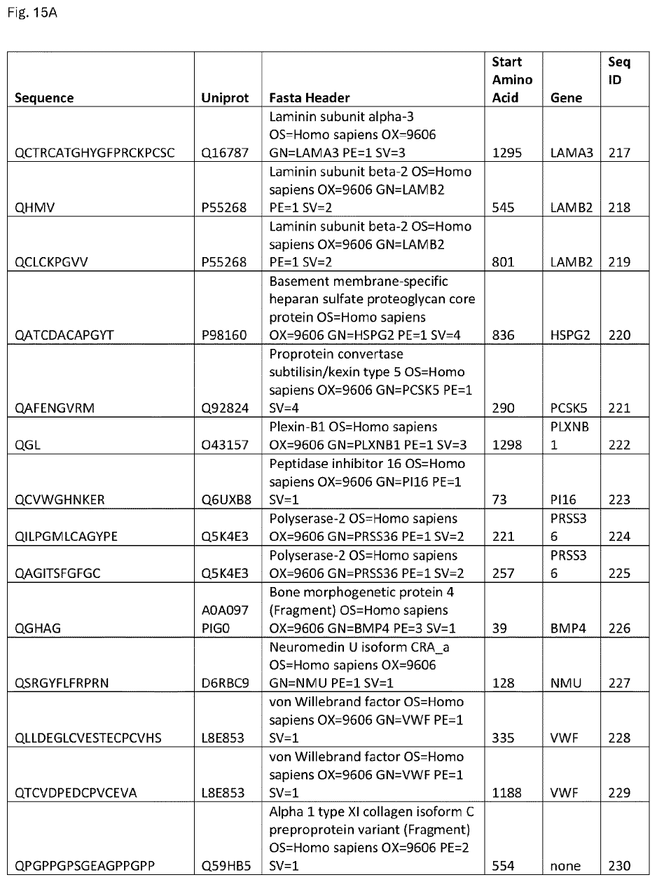

In some embodiments, the capped peptide may be from a variety of different species, including mammalian and non-mammalian species. In some embodiments, the capped peptide is a murine peptide. Examples of murine capped peptides of the invention include, but are not limited to, those listed in FIG. 14. In some embodiments, the capped peptide is a peptide listed in FIG. 14. In some embodiments, the capped peptide is a peptide listed in FIG. 14A. In some embodiments, the murine capped peptides may vary in size, ranging from 2 to 30 amino acid residues, such as 3 to 25 amino acid residues, and including 3 to 20 amino acid residues.

In some embodiments, the murine capped peptide may be modified with an N-terminal pyroglutamylation and C-terminal amidation from a sequence as shown in FIG. 14, or a sequence that is substantially the same as the sequence as shown in FIG. 14. By substantially the same as is meant a peptide having a region with a sequence that is 60% or greater, such as 75% or greater, such as 80% or greater, such as 85% or greater, such as 90% or greater, such as 95% or greater and including 98% or greater sequence identity with a sequence as shown in FIG. 14, e.g., as determined by BLAST using default settings.

In some embodiments, the murine capped peptide may be modified with an N-terminal pyroglutamylation and C-terminal amidation from a sequence as shown in FIG. 14A, or a sequence that has a sequence that is substantially the same as the sequence as shown in FIG. 14A. By substantially the same as is meant a peptide having a region with a sequence that is 60% or greater, such as 75% or greater, such as 80% or greater, such as 85% or greater, such as 90% or greater, such as 95% or greater and including 98% or greater sequence identity with a sequence as shown in FIG. 14A, e.g., as determined by BLAST using default settings.

In some embodiments, the murine capped peptide comprises a lysine residue. In some embodiments, the murine capped peptide comprises a lysine residue four or fewer residues away, including four residues away, three residues away, two residues away, and one residue away from the N-terminal pyroglutamylation modification. In some embodiments, the murine capped peptide comprises a glycine residue. In some embodiments, the murine capped peptide comprises a glycine residue four or fewer residues away, including four residues away, three residues away, two residues away, and one residue away from the N-terminal pyroglutamylation modification.

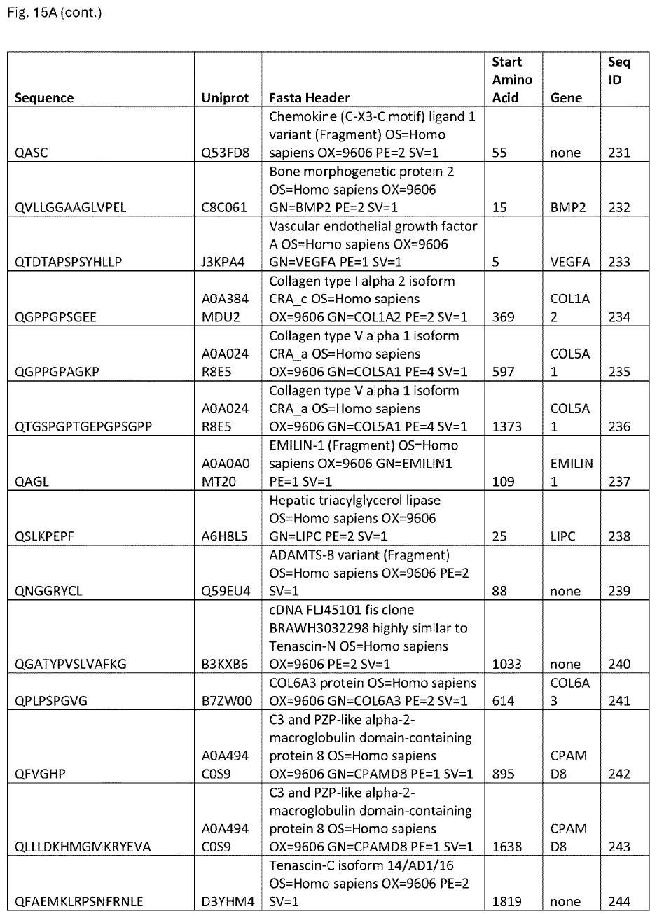

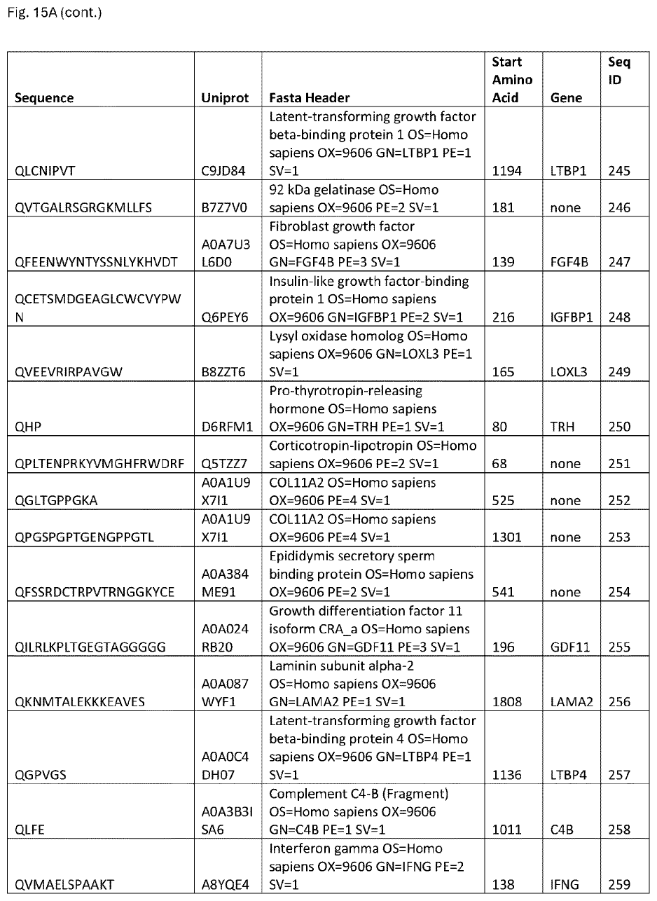

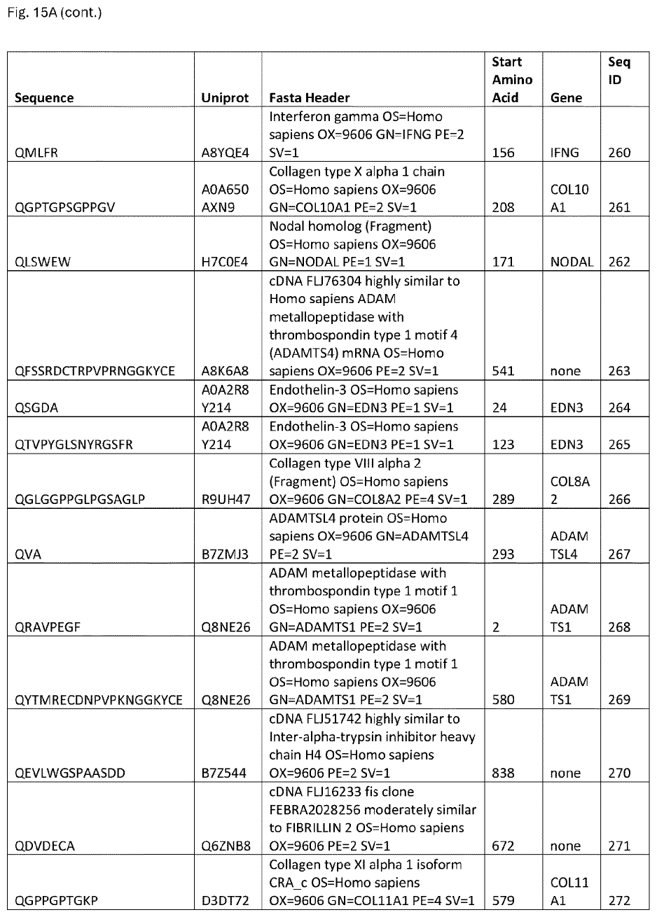

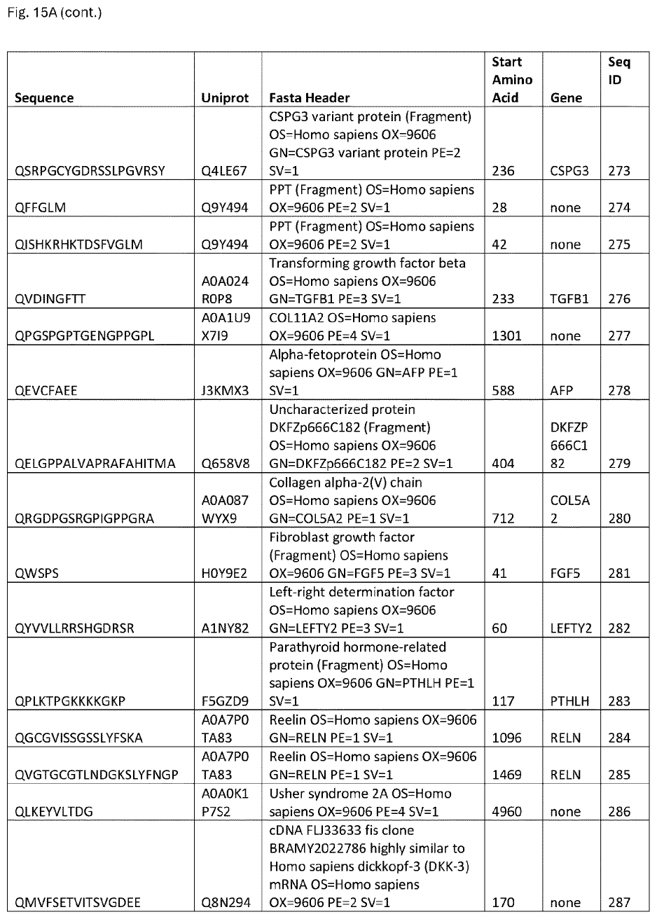

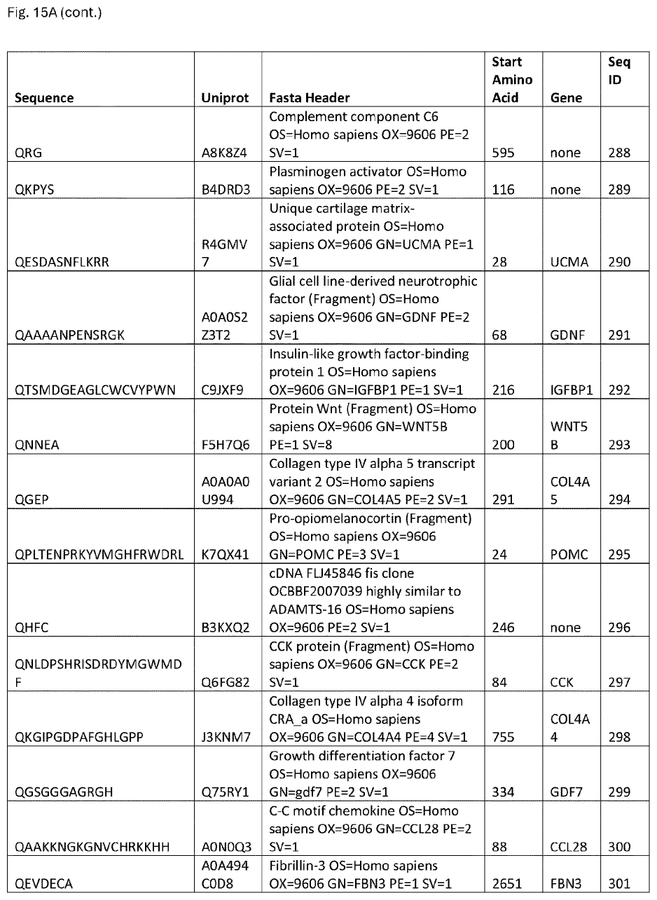

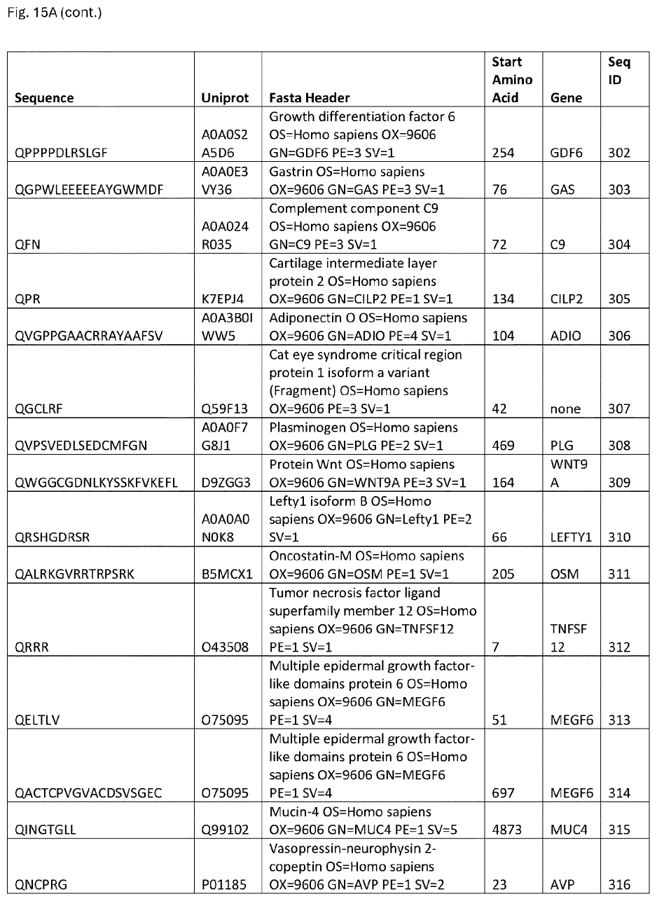

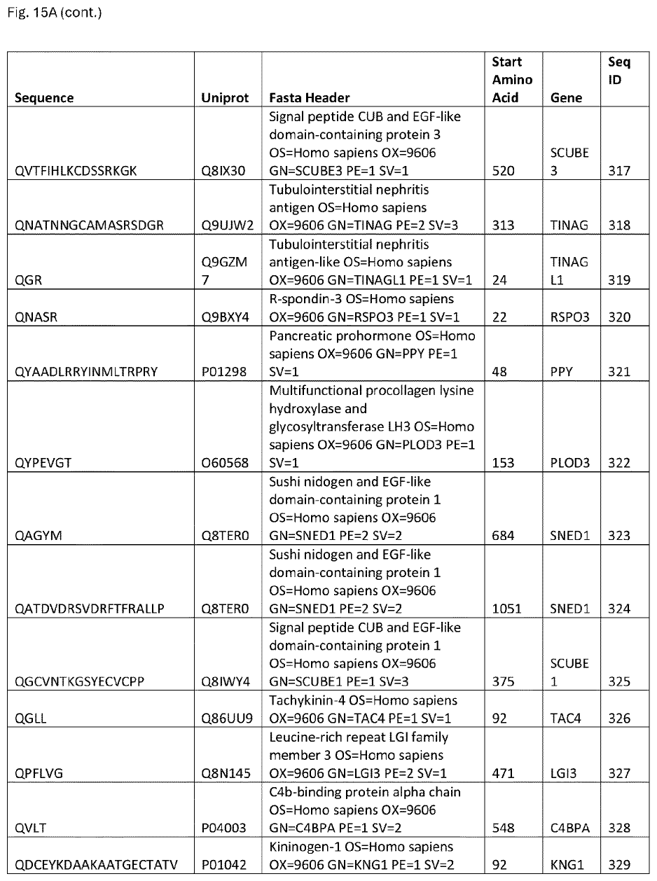

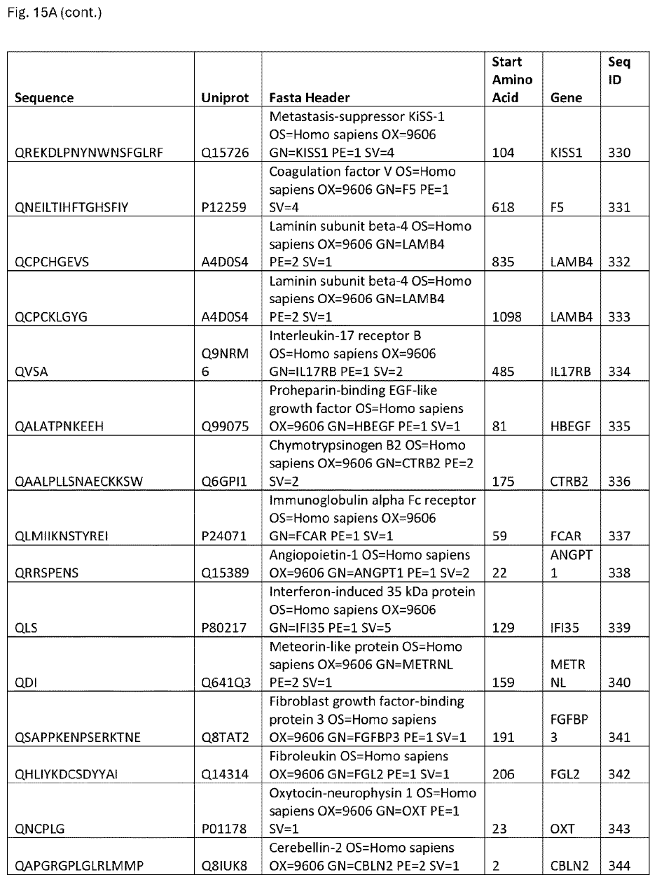

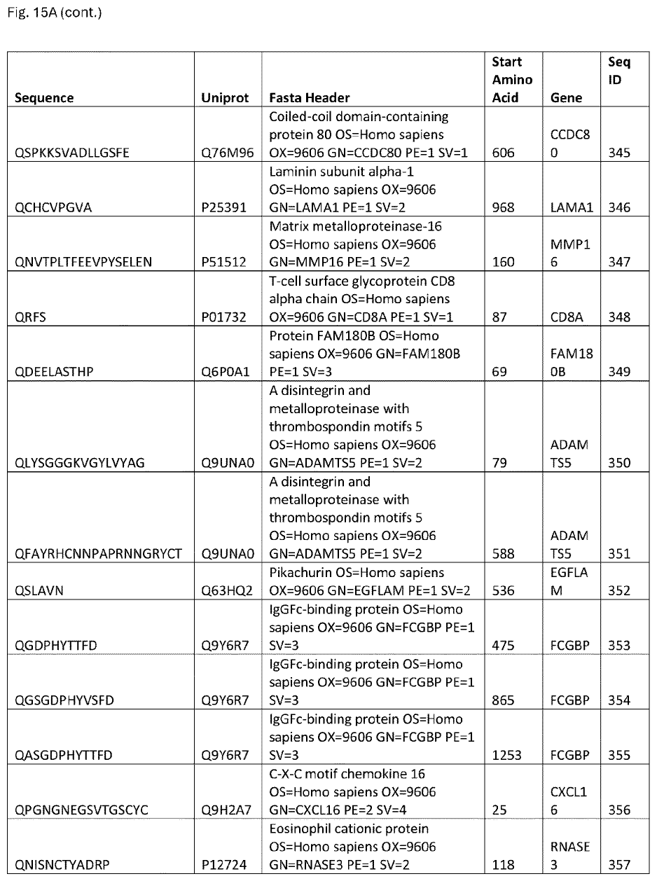

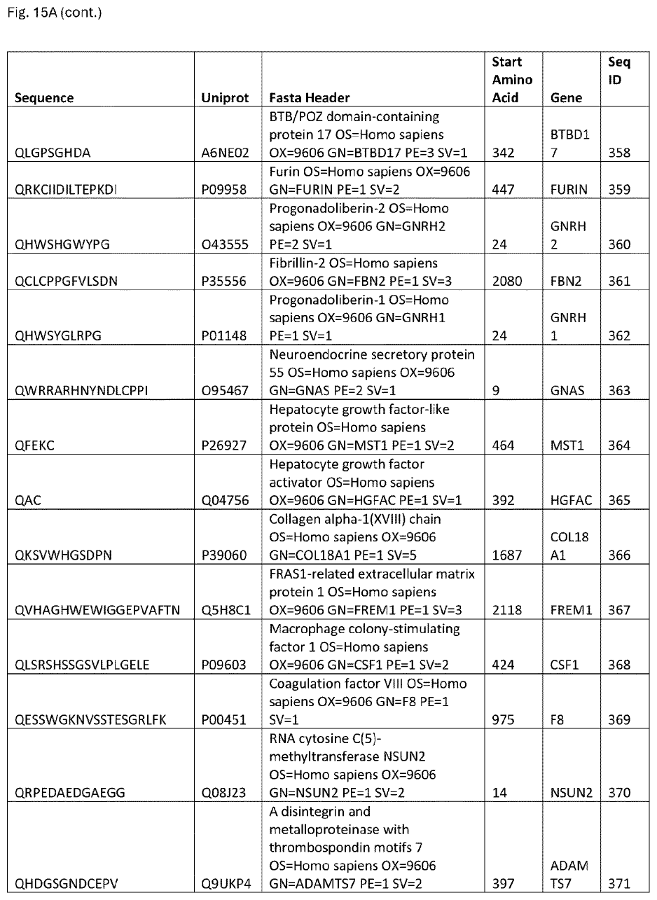

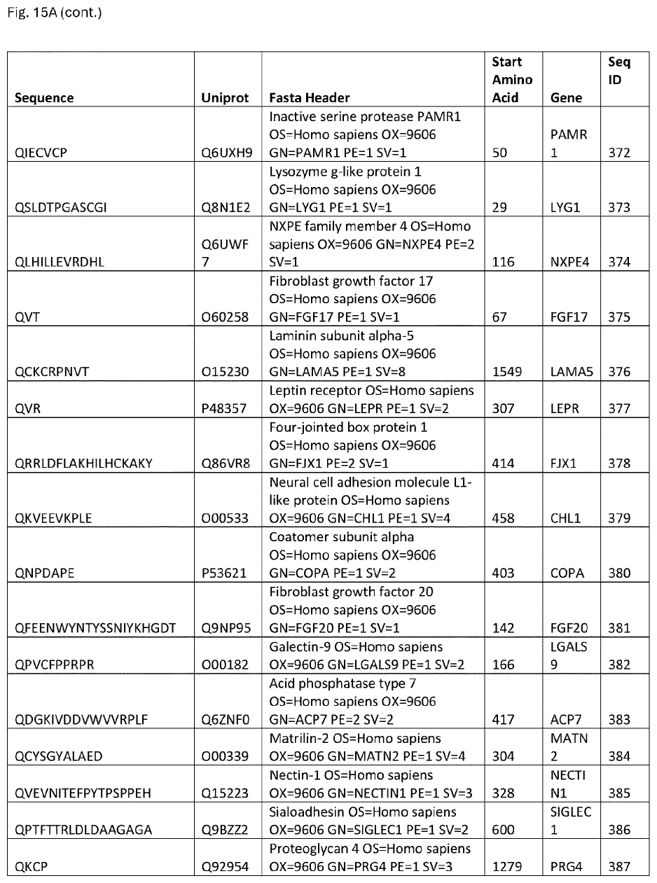

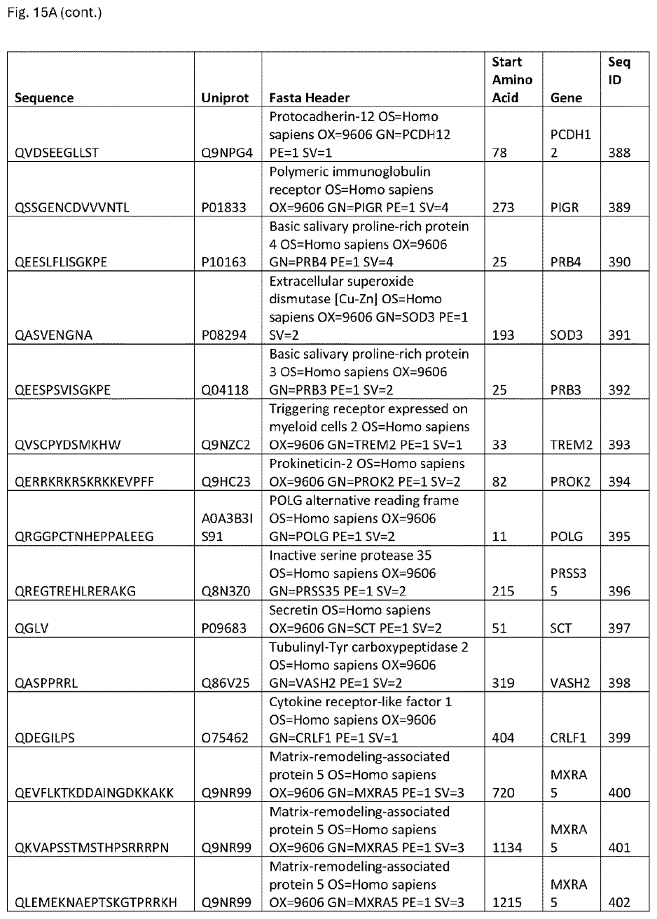

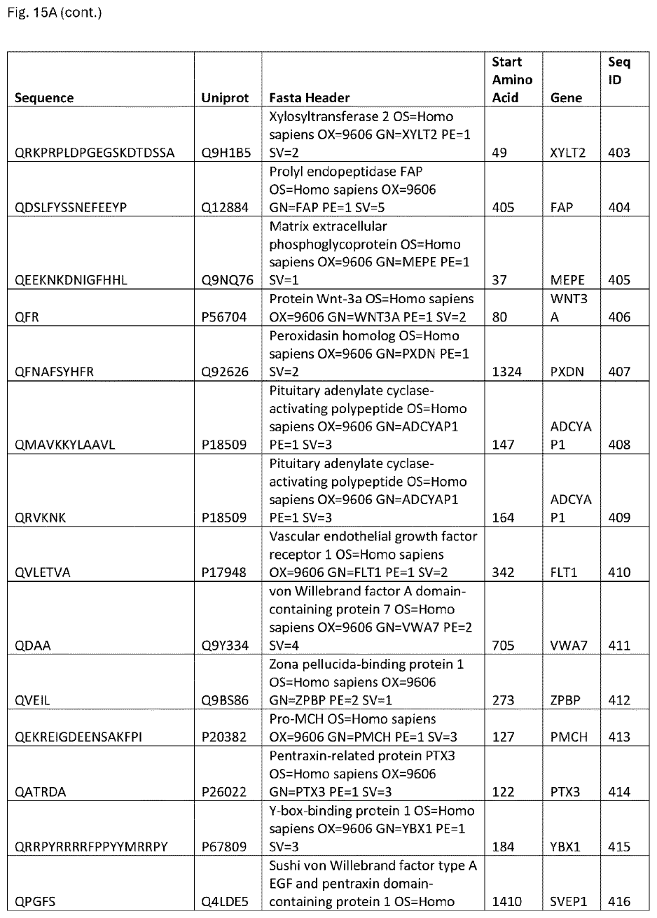

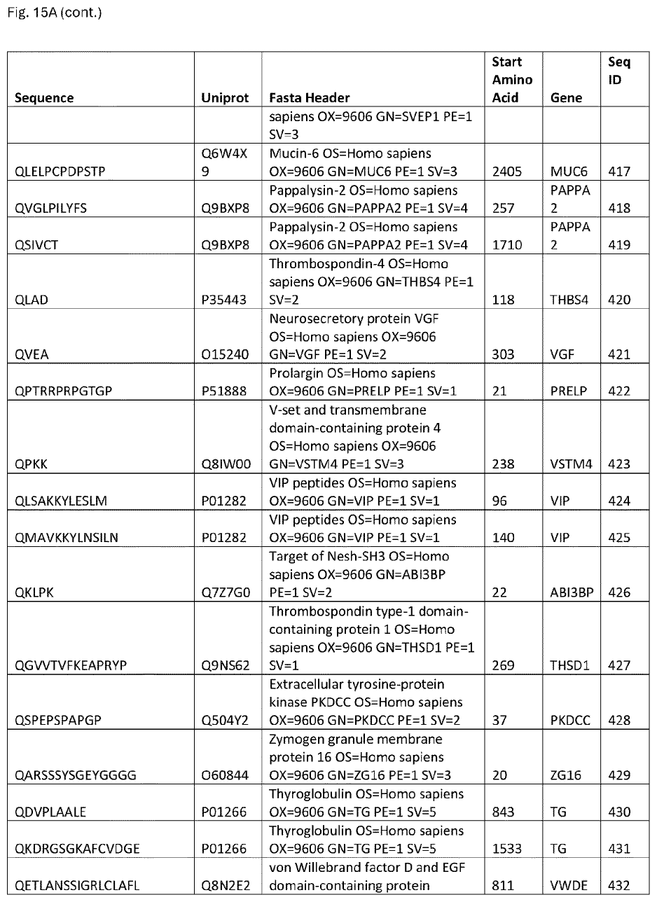

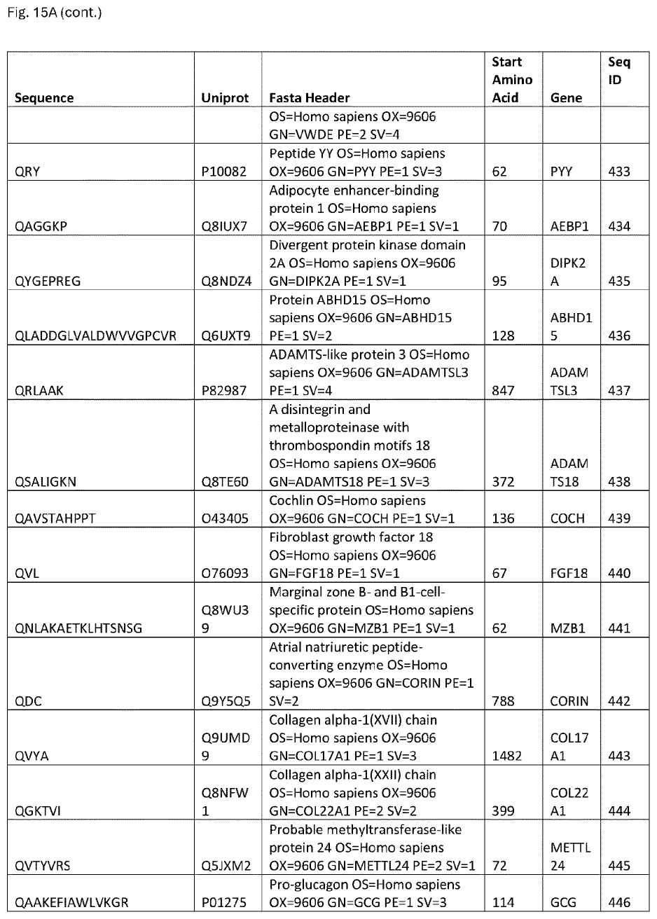

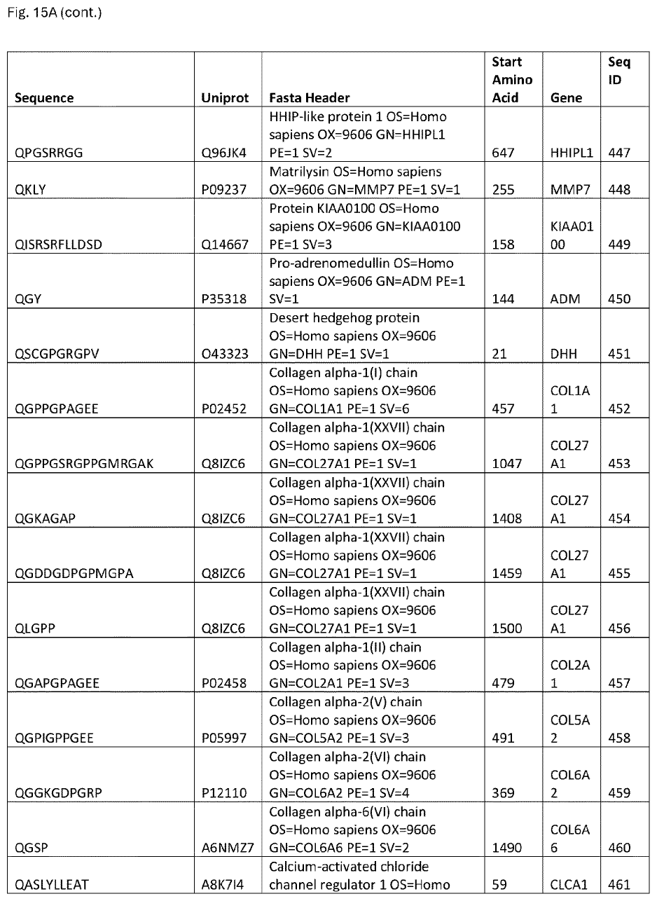

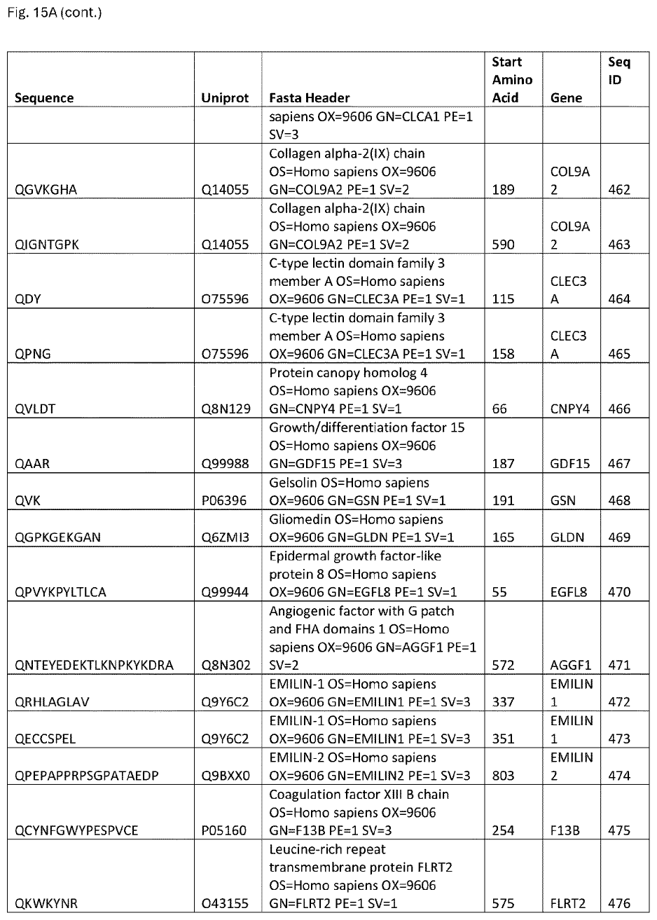

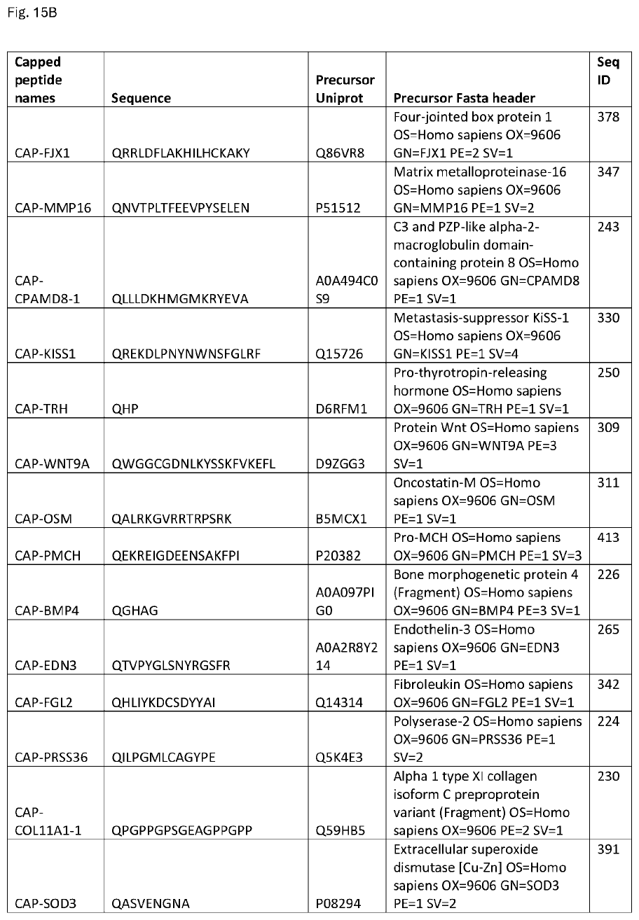

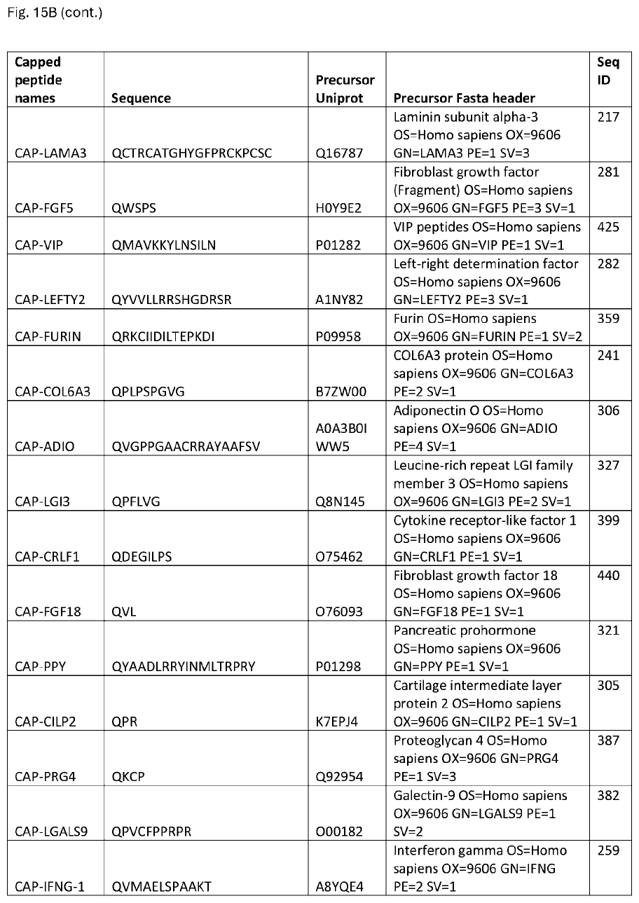

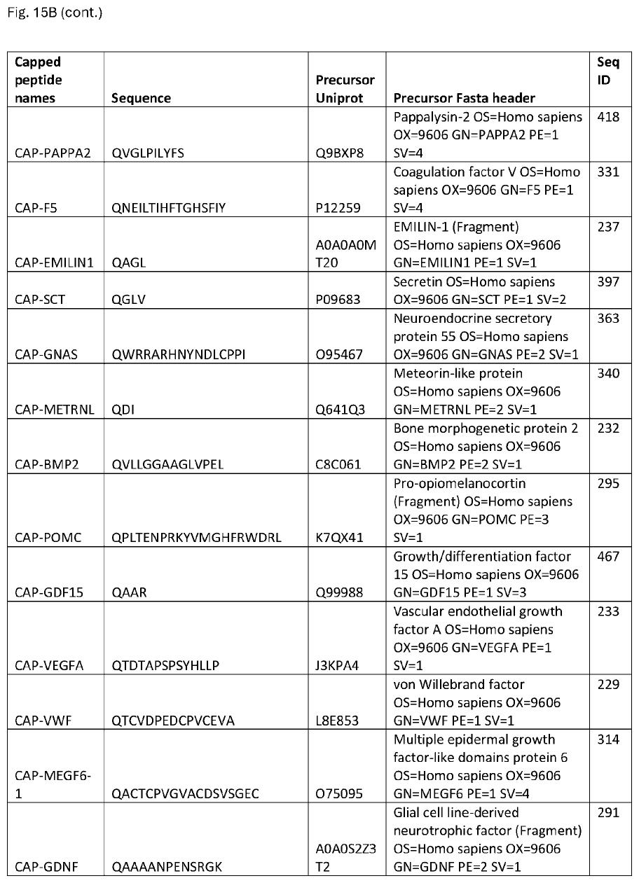

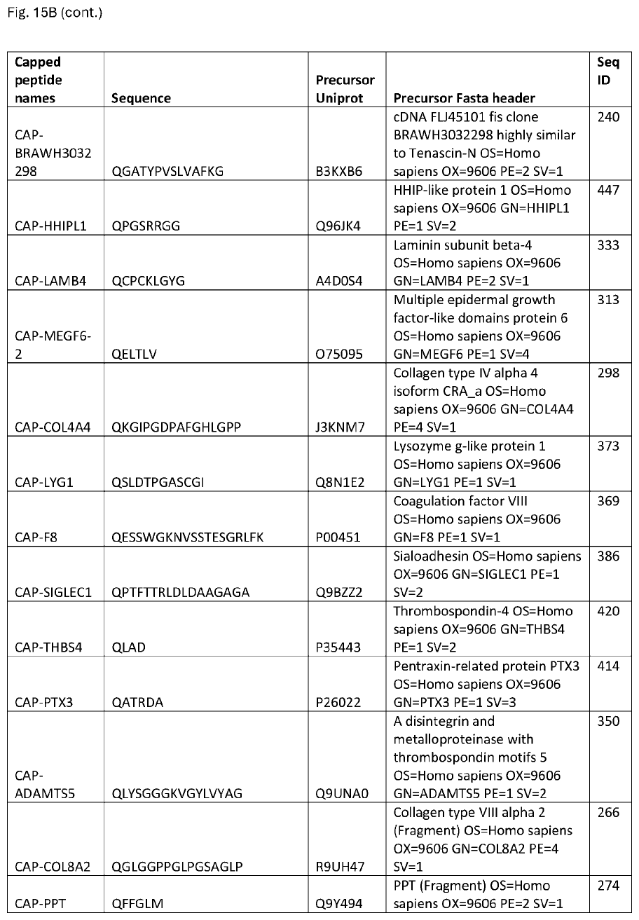

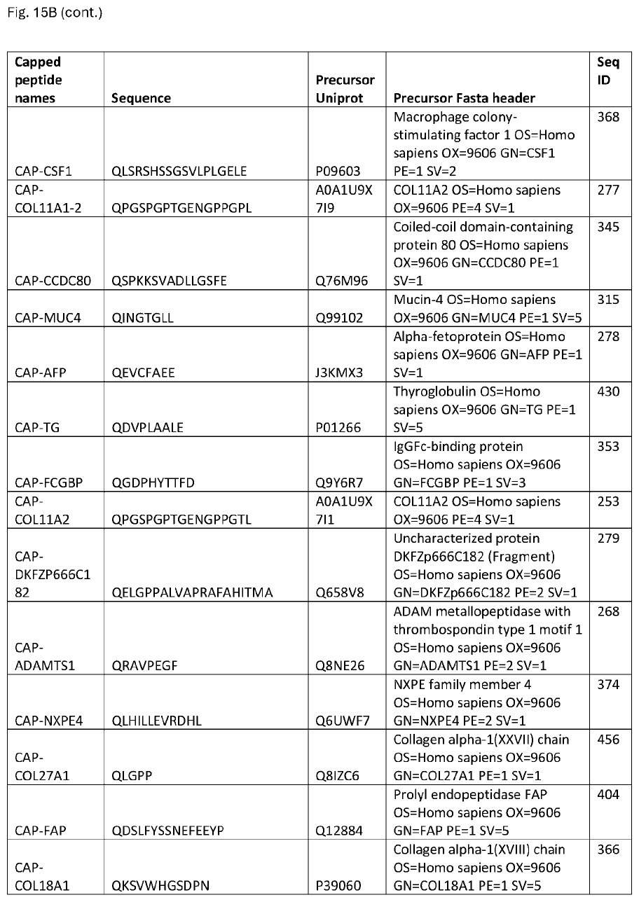

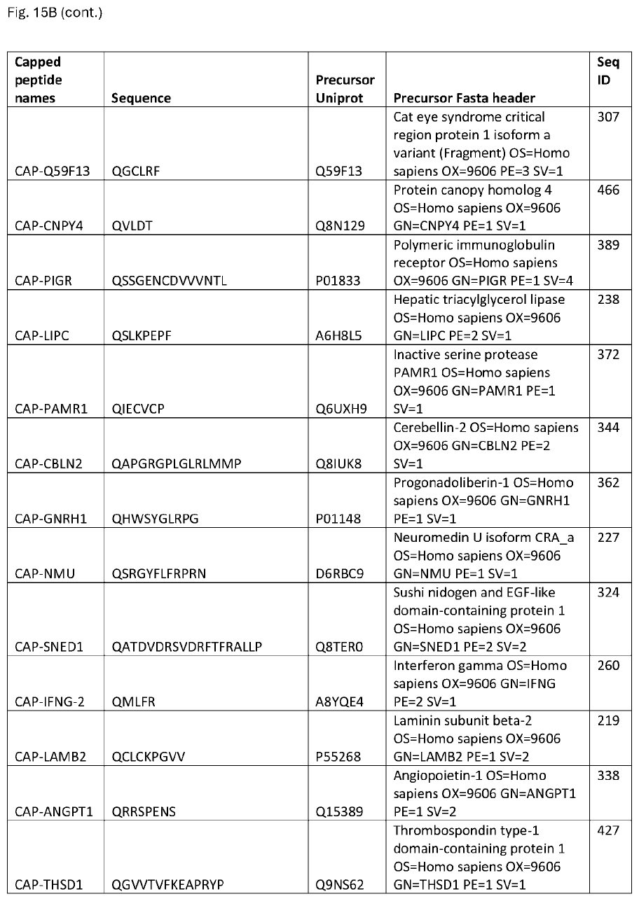

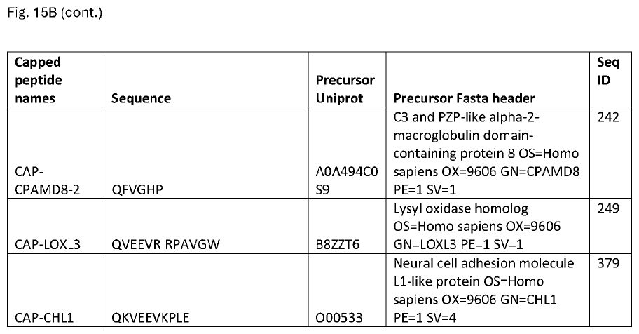

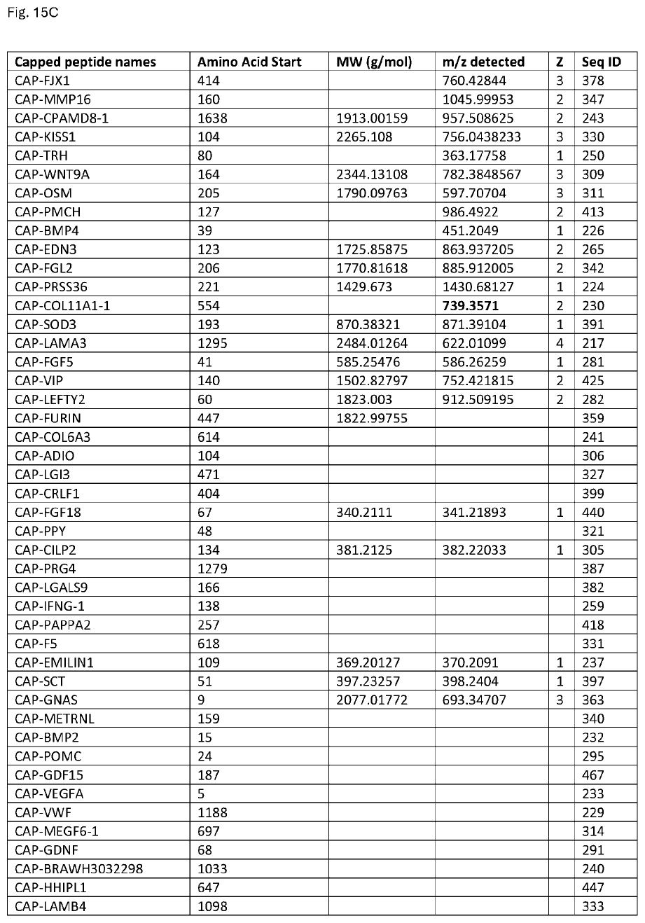

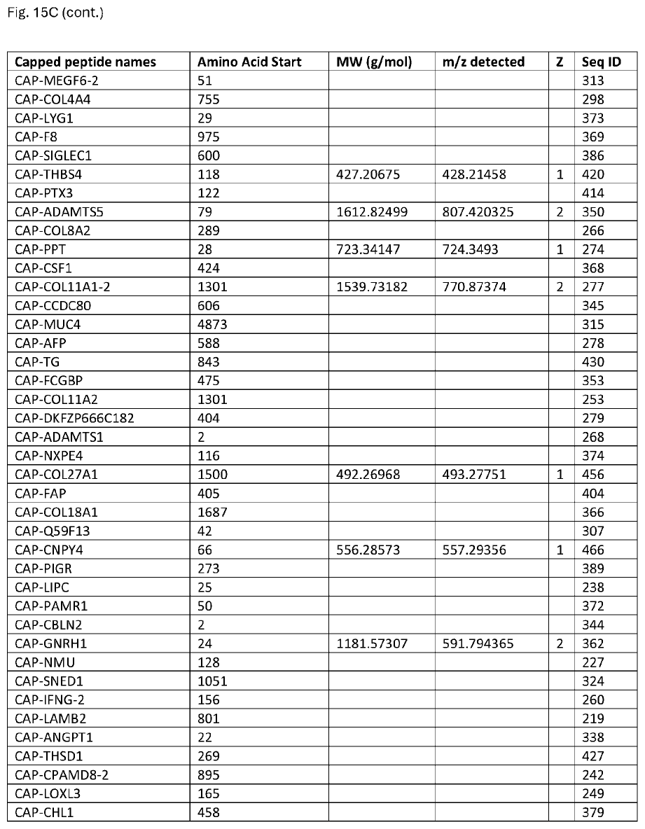

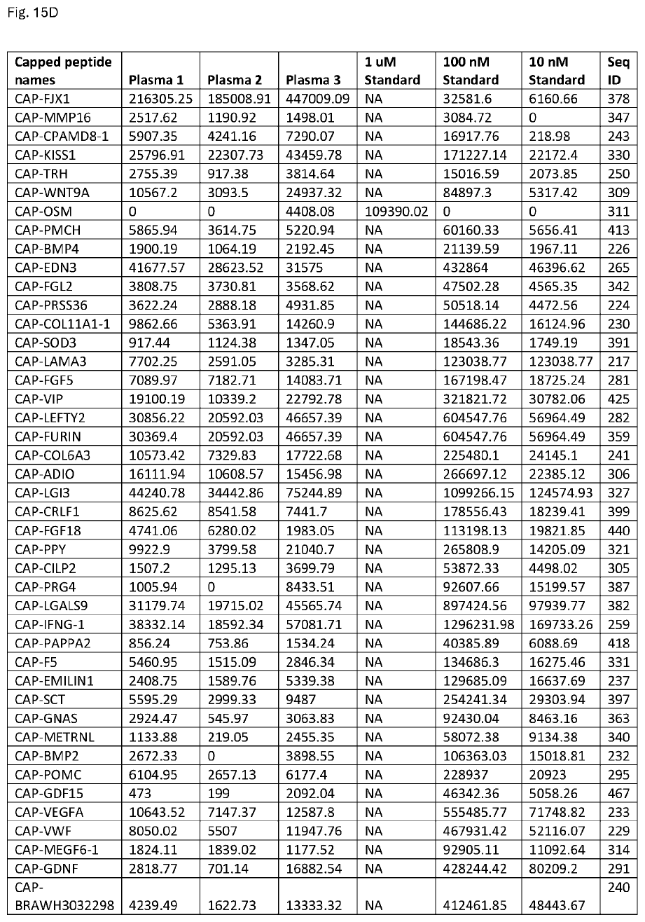

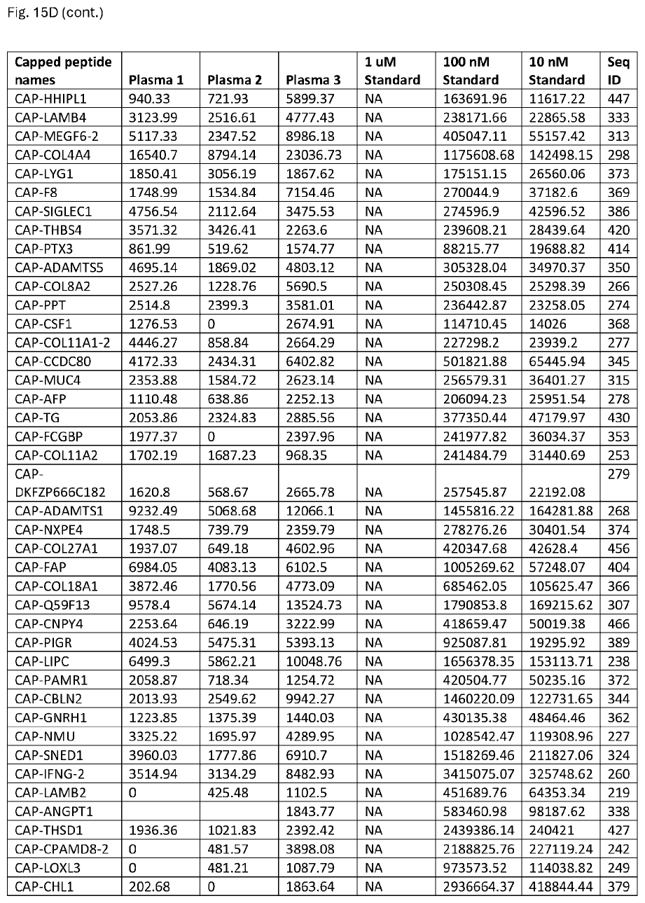

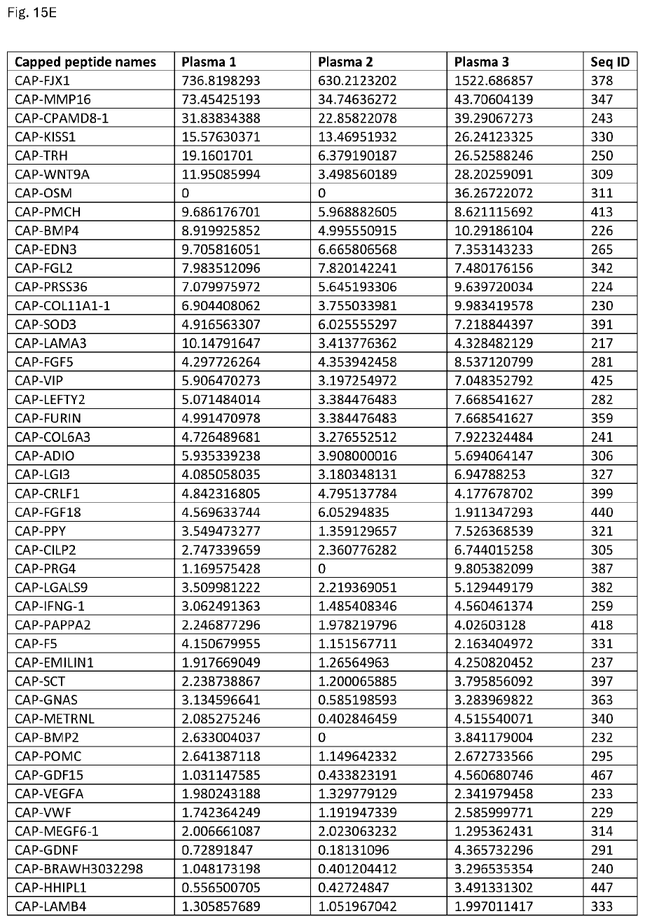

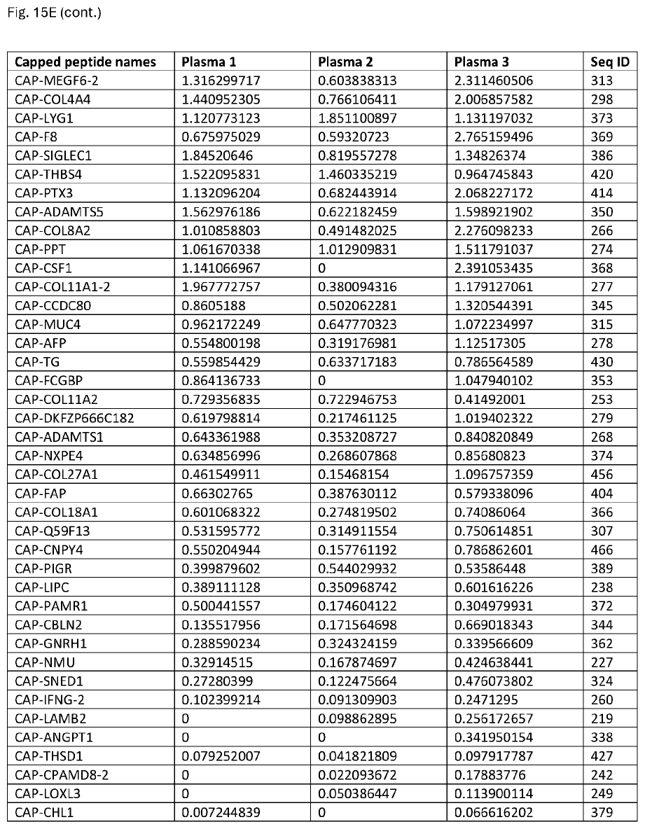

In some embodiments, the capped peptide is a human peptide. Examples of human capped peptides of the invention include, but are not limited to, those listed in FIG. 15. In some embodiments, the capped peptide is a peptide listed in FIG. 15. In some embodiments, the capped peptide is a peptide listed in FIG. 15A. In some embodiments, the human capped peptides may vary in size, ranging from 2 to 30 amino acid residues, such as 3 to 25 amino acid residues, and including 3 to 20 amino acid residues.

In some embodiments, the human capped peptide may be modified with an N-terminal pyroglutamylation and C-terminal amidation from a sequence as shown in FIG. 15, or a sequence that has a sequence that is substantially the same as the sequence as shown in FIG. 15. By substantially the same as is meant a peptide having a region with a sequence that is 60% or greater, such as 75% or greater, such as 80% or greater, such as 85% or greater, such as 90% or greater, such as 95% or greater and including 98% or greater sequence identity with a sequence as shown in FIG. 15, e.g., as determined by BLAST using default settings.

In some embodiments, the human capped peptide may be modified with an N-terminal pyroglutamylation and C-terminal amidation from a sequence as shown in FIG. 15A, or a sequence that has a sequence that is substantially the same as the sequence as shown in FIG. 15A. By substantially the same as is meant a peptide having a region with a sequence that is 60% or greater, such as 75% or greater, such as 80% or greater, such as 85% or greater, such as 90% or greater, such as 95% or greater and including 98% or greater sequence identity with a sequence as shown in FIG. 15A, e.g., as determined by BLAST using default settings.

In some embodiments, the human capped peptide comprises a lysine residue. In some embodiments, the human capped peptide comprises a lysine residue four or fewer residues away, including four residues away, three residues away, two residues away, and one residue away from the N-terminal pyroglutamylation modification. In some embodiments, the human capped peptide comprises a proline residue. In some embodiments, the murine capped peptide comprises a proline residue four or fewer residues away, including four residues away, three residues away, two residues away, and one residue away from the N-terminal pyroglutamylation modification. In some embodiments, the human capped peptide comprises a valine residue. In some embodiments, the human capped peptide comprises a valine residue four or fewer residues away, including four residues away, three residues away, two residues away, and one residue away from the N-terminal pyroglutamylation modification.

In some embodiments, the capped peptide of the present invention exhibits physiological activity. By physiological activity it is meant that the capped peptides exhibit a measurable biological response and/or change. Biological responses include, but are not limited to, molecular responses, cellular responses, tissue-specific responses, organ-specific responses, organism-specific responses and combinations thereof. Measurable biological responses are able to be detected and/or quantified as a change from baseline activity.

Physiological activity may include mammalian tachykinin receptor agonist activity. Tachykinins are a family of peptides that perturb many biological processes via interaction with tachykinin receptors. There are three known mammalian tachykinin receptors: NK₁, NK₂, and NK₃. Tachykinins have been reported to participate in various physiological processes including, but not limited to, processes in the nervous, immune, gastrointestinal, respiratory, urogenital and dermal systems (Steinhoff, M. S., Physiol. Rev. 2014 94:265-301). Agonists of tachykinins can bind to tachykinin receptors and thereby elicit and/or modify tachykinin-related responses. In certain embodiments, the capped peptide exhibits mammalian tachykinin receptor agonist activity. In certain embodiments, the capped peptide exhibits mammalian tachykinin receptor NK₁ agonist activity. In certain embodiments, the capped peptide exhibits mammalian tachykinin receptor NK₂ agonist activity. In certain embodiments, the capped peptide exhibits mammalian tachykinin receptor NK₃ agonist activity. In certain embodiments, the capped peptide exhibits murine tachykinin receptor agonist activity. In certain embodiments, the capped peptide exhibits human tachykinin receptor agonist activity.

Physiological activity may include activating metabolic activity, wherein activation of metabolic activity means there is an increase in metabolic activity. Activating metabolic activity includes all of the processes that are necessary for breaking down compounds (i.e., catabolic processes) to cause an increase in energy consumption. The increase in energy consumption may occur in a cell, organ, organism, or combinations thereof. Increases in metabolic activity (i.e., activating metabolic activity) may result in increased caloric consumption. Activating metabolic activity may also be characterized as, for example, increased food intake that is not accompanied by an increase in body weight, decreased body weight that is not accompanied by an increase in food intake, increased in oxygen consumption that is not accompanied by an increase in food intake, and/or combinations thereof. In some embodiments, the capped peptide exhibits activating metabolic activity. In some embodiments, the capped peptide exhibits activating metabolic activity in humans. In some embodiments, the capped peptide exhibits activating metabolic activity in mice.

Physiological activity may include hypophagic activity. Hypophagia is defined as the reduced ingestion of food. Hypophagic activity is characterized by decreased ingestion of food, decreased consumption of food, loss of appetite, not feeling hungry, and combinations thereof. In some embodiments, the capped peptide exhibits hypophagic activity. In some embodiments, the capped peptide exhibits human hypophagic activity. In some embodiments, the capped peptide exhibits murine hypophagic activity.

Physiological activity may include growth hormone secretagogue activity. A secretagogue is an agent that promotes the secretion of a molecule. As such, a growth hormone secretagogue is an agent that promotes the secretion of growth hormone. Growth hormone is a peptide hormone that stimulates growth, cell reproduction, and cell regeneration in mammals, including humans. In some embodiments, the capped peptide exhibits growth hormone secretagogue activity. In some embodiments, the capped peptide exhibits human growth hormone secretagogue activity. In some embodiments, the capped peptide exhibits murine growth hormone secretagogue activity.

In certain embodiments, the capped peptide is CAP-TAC1 comprising the sequence pGlu-FFGLM-NH2 or a peptide having a region with a sequence that is 60% or greater, such as 75% or greater, such as 80% or greater, such as 85% or greater, such as 90% or greater, such as 95% or greater and including 98% or greater sequence identity with CAP-TAC1. In certain embodiments, CAP-TAC1 or a peptide having a region with a sequence that is 60% or greater, such as 75% or greater, such as 80% or greater, such as 85% or greater, such as 90% or greater, such as 95% or greater and including 98% or greater sequence identity with CAP-TAC1 exhibits tachykinin receptor agonist activity. In certain embodiments, CAP-TAC1 or a peptide having a region with a sequence that is 60% or greater, such as 75% or greater, such as 80% or greater, such as 85% or greater, such as 90% or greater, such as 95% or greater and including 98% or greater sequence identity with CAP-TAC1 exhibits activating metabolic activity. In certain embodiments, CAP-TAC1 or a peptide having a region with a sequence that is 60% or greater, such as 75% or greater, such as 80% or greater, such as 85% or greater, such as 90% or greater, such as 95% or greater and including 98% or greater sequence identity with CAP-TAC1 exhibits tachykinin receptor agonist activity and activating metabolic activity.

In certain embodiments, the capped peptide is CAP-GDF15 comprising the sequence pGlu-LELRLRVAAGR-NH2 or a peptide having a region with a sequence that is 60% or greater, such as 75% or greater, such as 80% or greater, such as 85% or greater, such as 90% or greater, such as 95% or greater and including 98% or greater sequence identity with CAP-GDF15. In certain embodiments, CAP-GDF15 or a peptide having a region with a sequence that is 60% or greater, such as 75% or greater, such as 80% or greater, such as 85% or greater, such as 90% or greater, such as 95% or greater and including 98% or greater sequence identity with CAP-GDF15 exhibits hypophagic activity.

In certain embodiments, the capped peptide is CAP-WNT9A comprising the sequence pGlu-WGGCGDNLKYSSKFVEFL-NH2 or a peptide having a region with a sequence that is 60% or greater, such as 75% or greater, such as 80% or greater, such as 85% or greater, such as 90% or greater, such as 95% or greater and including 98% or greater sequence identity with CAP-WNT9A. In certain embodiments, CAP-WNT9A or a peptide having a region with a sequence that is 60% or greater, such as 75% or greater, such as 80% or greater, such as 85% or greater, such as 90% or greater, such as 95% or greater and including 98% or greater sequence identity with CAP-WNT9A exhibits growth hormone secretagogue activity.

### Methods of Use

The present disclosure includes methods of using capped peptides. Aspects of such methods include administering a capped peptide to a subject in need thereof, wherein the capped peptide comprises an N-terminal pyroglutamylation modification and a C-terminal amidation modification, and is not Thyrotropin-releasing hormone (TRH) or (Gonadotropin-releasing hormone (GnRH)/luteinizing hormone-releasing hormone (LHRH).

As reviewed above, in some embodiments, capped peptides of the present invention exhibit physiological activity. Physiological activity includes, but is not limited to, mammalian tachykinin receptor agonist activity, activating metabolic activity, hypophagic activity and growth hormone secretagogue activity. Such physiological activities may be beneficial in the treatment of various disorders, diseases and conditions. Aspects of the disclosure include methods of using capped peptides to treat a subject in need thereof. In some embodiments, the method is a method of treating a subject for a disorder, disease or condition.

### Methods of Treating Neurological Disorders

In some embodiments, the method is a method of treating a subject for a neurological disorder. Neurological disorders include disorders of the central nervous system and peripheral nervous system. In other words, neurological disorders include disorders of the brain, spinal cord, cranial nerves, peripheral nerves, nerve roots, autonomic nervous system, neuromuscular junction and muscles. Disorders of the brain include brain dysfunction such as apraxia (i.e., brain dysfunction related to movement), agnosia (i.e., brain dysfunction related to processing sensory information), amnesia (i.e. brain dysfunction related to memory), aphasia (i.e. brain dysfunction related to language) and dysarthria (i.e. brain dysfunction related to speech). Neurological disorders may be pain-related neurological disorders such as migraines, headaches, back pain, complex regional pain syndrome, fibromyalgia and chronic pain. Neurological disorders may be psychiatric disorders (i.e., mental disorders). Psychiatric disorders include anxiety and anxiety disorders, depression and depressive disorders, mood disorders, addiction and addictive disorders, and psychosis and psychotic disorders. Psychotic disorders include Schizophrenia. Other examples of neurological disorders include, but are not limited to, dysautonomia, multiple system atrophy, epilepsy, Parkinson's disease, Tourette's syndrome, amyotrophic lateral sclerosis, multiple sclerosis, pruritus, Alzheimer's disease and stroke.

As reviewed above, tachykinins and tachykinin-receptors participate in processes within the nervous system. As such, antagonism of tachykinin receptors is a method to control neurological disorders or symptoms thereof. In some cases, neurological disorders or symptoms of neurological disorders may be improved upon treatment with a capped peptide that exhibits mammalian tachykinin receptor agonist activity. In some embodiments, the method is a method of using capped peptides, wherein the capped peptide exhibits mammalian tachykinin receptor agonist activity, to treat a subject for a neurological disorder. In certain embodiments, the neurological disorder may include, but is not limited to, a depressive disorder, anxiety disorder, chronic pain, addiction, pruritus, psychosis or schizophrenia.

### Methods of Treating Inflammatory Disorders

In some embodiments, the method is a method of treating a subject for an inflammatory disorder. Inflammation is a biological response to harmful stimuli such as pathogens, damaged cells, or irritants. As such, a disorder of inflammation (i.e. an inflammatory disorder) is one wherein inflammation is dysregulated within the organism. Inflammation can present as heat, pain, redness, swelling and/or loss of function. Inflammation leads to a shift in the type of cells and/or signaling molecules present at the inflammation site. For example, inflammation may result in an increase of leukocytes and cytokines at the inflammation site. Inflammatory disorders include disorders wherein too little inflammation occurs and disorders wherein too much inflammation occurs. Inflammatory disorders include acute inflammatory disorders (i.e., the initial response to harmful stimuli) and chronic inflammatory disorders (i.e. prolonged inflammation). Acute inflammation may last for hours to days. Acute inflammatory diseases include, but are not limited to, ileus and appendicitis. Chronic inflammation may last for one month to one year, or even longer than a year. Chronic inflammatory disorders include, but are not limited to, irritable bowel syndrome, inflammatory bowel disease, Crohn's disease, asthma, colitis and fibrosis.

Tachykinins have been shown to be potent mediators of vasodilation, plasma extravasation, inflammatory cell recruitment and pain (Canning, B. J., J. Allergy Clin. Immunol. 1997. 99:579-582). As such, antagonism of tachykinin receptors is a method to control inflammatory responses, as well as inflammatory disorders and symptoms thereof. In some cases, inflammatory disorders or symptoms of inflammatory disorders may be improved upon treatment with a capped peptide that exhibits mammalian tachykinin receptor agonist activity. In some embodiments, the method is a method of using capped peptides, wherein the capped peptide exhibits mammalian tachykinin receptor agonist activity, to treat a subject for an inflammatory disorder. In certain embodiments, the inflammatory disorder may include, but is not limited to, irritable bowel syndrome, inflammatory bowel disease, asthma, colitis, ileus or fibrosis.

Methods of Treating Nausea and/or Vomiting

In some embodiments, the method is a method of treating a subject for nausea and/or vomiting. Nausea is a sensation that often includes the urge to vomit. Nausea and vomiting are symptoms resulting from a myriad of causes. Causes of nausea and vomiting include, but are not limited to, gastrointestinal disorders, food poisoning, motion sickness, dizziness, psychiatric disorders, low blood sugar, dehydration, lack of sleep, chemotherapy, anesthesia, and pregnancy. Nausea and vomiting are common side effects of chemotherapy treatment. Chemotherapy-induced nausea and vomiting includes acute (i.e., within the first 24 hours after chemotherapy), delayed (i.e., after 24 hours of chemotherapy), anticipatory (i.e., before chemotherapy dose administration but after prior cycles of chemotherapy), breakthrough (i.e. after nausea and vomiting treatment has already been administered), and chronic nausea and vomiting. Post-operative nausea and vomiting is a common side effect resulting from administration of anesthesia. Post-operative nausea and vomiting can result from administration of a variety of anesthetic agents including, but not limited to, nitrous oxide, ether, cyclopropaneetomidate, ketamine, propofol, opioids, and combinations thereof.

Tachykinins play a role in regulating nausea and vomiting. Specifically, tachykinin receptor NK₁ has been implicated in controlling nausea and vomiting. Antagonism of tachykinin receptors may treat nausea and vomiting. In some embodiments, the method is a method of using capped peptides, wherein the capped peptide exhibits mammalian tachykinin receptor agonist activity, to treat a subject for nausea and vomiting. In some embodiments, the method is a method of using capped peptides, wherein the capped peptide exhibits mammalian tachykinin receptor NK₁ agonist activity, to treat a subject for nausea and vomiting. In certain embodiments, the method is used to treat chemotherapy-induced nausea and vomiting. In certain embodiments, the method is used to treat postoperative nausea and vomiting.

### Methods of Treating Obesity

In some embodiments, the method is a method of treating a subject for obesity or an obesity-related disorder. Obesity is defined as an excess of body fat relative to lean body mass. A subject is generally defined as obese if the subject has a body mass index of 30 kg/m² or greater. Obesity is often caused by excessive food intake coupled with limited energy expenditure and/or lack of physical exercise. Obesity increases the likelihood of various disorders. Obesity-related disorders may include, but are not limited to, hypertension, dyslipidemia, mellitus, atherosclerosis, gout, rheumatism, arthritis, type 2 diabetes, coronary heart disease, stroke, gallbladder disease, liver disease, sleep apnea and pain.

As reviewed above, activating metabolic activity increases energy consumption, resulting in a decrease in body weight. In some embodiments, the method is a method of using capped peptides, wherein the capped peptide exhibits activating metabolic activity, to treat a subject for obesity or an obesity-related disorder.

Hypophagic activity reduces ingestion of food, resulting in a decrease in body weight. In some embodiments, the method is a method of using capped peptides, wherein the capped peptide exhibits hypophagic activity, to treat a subject for obesity or an obesity-related disorder. In certain embodiments, the subject has a body mass index greater than 30 kg/m².

Efficacy of treatment for obesity can be readily determined by weight loss, for example the reduced food intake observed with administration is associated with weight loss, e.g., loss of 1% body weight, 2.5%, 5%, 7.5%, 10%, 12.5%, 15%, 17.5%, 20%, 22.5%, 25%, 27.5%, 30% or more, depending on the initial weight of the subject. Methods for measuring weight loss can be determined by weighing the subject.

### Methods of Treating Growth Hormone Deficiency and Disorders Thereof.

In some embodiments, the method is a method of treating a subject for a growth hormone deficiency. Growth hormone is produced by the pituitary gland and stimulates growth, cell reproduction and cell regeneration throughout the body. As such, growth hormone is required for human development. Deficiency of growth hormone in adolescents results in slow and limited growth. In general, growth hormone has many effects on the body including, but not limited to, increasing calcium retention, increasing muscle mass, promoting lipolysis, increasing protein synthesis, stimulating growth of organs, stimulating growth of the brain, regulating homeostasis, reducing liver uptake of glucose, stimulating the immune system, and inducing insulin resistance. Lack of growth hormone or growth hormone deficiency results in a variety of disorders including, but not limited to, depression, anxiety, obesity, cardiac dysfunction, reduced energy, low libido, impaired concentration, memory loss, loss of hair growth, decreased muscle mass, insulin resistance and loss of thermoregulation. Growth hormone deficiency related-disorders may include, but are not limited to dwarfism, pituitary dwarfism, osteoporosis and dyslipidemia.

As reviewed above, growth hormone secretagogue activity promotes the secretion of growth hormone. Increased levels of growth hormone can have beneficial effects in treating growth hormone deficiency or disorders of growth hormone deficiency. In some embodiments, the method is a method of using capped peptides, wherein the capped peptide exhibits growth hormone secretagogue activity, to treat a subject for growth hormone deficiency or a growth hormone deficiency-disorder.

Efficacy of treatment for growth hormone deficiency or growth hormone deficiency-related disorders can be readily determined by measuring the change in the levels of growth hormone in a subject, e.g., a 1%, 2.5%, 5%, 7.5%, 10%, 12.5%, 15%, 17.5%, 20%, 22.5%, 25%, 27.5%, 30% or more, increase in growth hormone levels. Changes in the levels of growth hormone in a subject can be measured in blood and/or urine samples. Growth hormone levels can be measured using a variety of different methods including, but not limited to, immunoassays such as ELISA, gas chromatography mass spectrometry (GCMS) and liquid chromatography mass spectrometry (LC-MS/MS).

### Methods of Administration

Methods of administration may be carried out by any suitable means, including topical, oral, parenteral, intrapulmonary, and intranasal. Parenteral infusions include intramuscular, intravenous (bolus or slow drip), intraarterial, intraperitoneal, intrathecal or subcutaneous administration. An agent can be administered in any manner which is medically acceptable. This may include injections, by parenteral routes such as intravenous, intravascular, intraarterial, subcutaneous, intramuscular, intratumor, intraperitoneal, intraventricular, intraepidural, or others as well as oral, nasal, ophthalmic, rectal, or topical. Sustained release administration is also included in the disclosure, by such means as depot injections or erodible implants.

Dosage and frequency of dosing may vary depending on the half-life of the agent (e.g., a capped peptide) in the patient. It will be understood by one of skill in the art that such guidelines will be adjusted for the molecular weight of the active agent, the clearance from the blood, the mode of administration, and other pharmacokinetic parameters. The dosage may also be varied for localized administration, e.g., intranasal, inhalation, etc., or for systemic administration, e.g., i.m., i.p., i.v., oral, and the like.

### Pharmaceutical Compositions

Aspects of the present disclosure include pharmaceutical compositions comprising a capped peptide and a pharmaceutically acceptable delivery vehicle. Pharmaceutically acceptable delivery vehicles may include one or more vehicles and/or carriers. The vehicle(s) or carrier(s) must be “acceptable” in the sense of being compatible with the other ingredients of the formulation and not deleterious to the recipient thereof. A pharmaceutically acceptable delivery vehicle is selected on the basis of the chosen route of administration and standard pharmaceutical practice. Proper formulation is dependent upon the route of administration chosen. In instances where more than one vehicle and/or carrier is used, the vehicles and/or carriers must be “pharmaceutically acceptable” in the sense of being compatible with the other ingredients of the formulation and not injurious to the subject. Any of the well-known techniques, carriers, and excipients may be used as suitable and as understood in the art; e.g., in Remington's Pharmaceutical Sciences. The pharmaceutical compositions disclosed herein may be manufactured in any manner known in the art.

As noted above, an agent, such as a capped peptide, can be formulated with an a pharmaceutically acceptable delivery vehicle (one or more organic or inorganic ingredients, natural or synthetic, with which a subject agent is combined to facilitate its application). A suitable delivery vehicle includes sterile saline although other aqueous and non-aqueous isotonic sterile solutions and sterile suspensions known to be pharmaceutically acceptable are known to those of ordinary skill in the art. An “effective amount” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

An agent, such as a capped peptide, can be administered as a pharmaceutical composition comprising a pharmaceutically acceptable excipient. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Compounds useful for co-administration with the active agents, such as capped peptides, of the invention can be made by methods known to one of ordinary skill in the art. As used herein, “methods known to one of ordinary skill in the art” may be identified through various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

Compounds may also be purchased. Such “commercially available” compounds may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

In pharmaceutical dosage forms, the active agents and/or other compounds may be administered in the form of their pharmaceutically acceptable salts, or they may also be used alone or in appropriate association, as well as in combination with other pharmaceutically active compounds. The agents may be combined, as previously described, to provide a cocktail of activities. The following methods and excipients are exemplary and are not to be construed as limiting the invention.

Formulations are typically provided in a unit dosage form, where the term “unit dosage form,” refers to physically discrete units suitable as unitary dosages for human subjects, each unit containing a predetermined quantity of active agent in an amount calculated sufficient to produce the desired effect in association with a pharmaceutically acceptable diluent, carrier or vehicle. The specifications for the unit dosage forms of the present invention depend on the particular complex employed and the effect to be achieved, and the pharmacodynamics associated with each complex in the host.

In some embodiments a unit dose is at least about 0.1 mg/kg, at least about 0.5 mg/kg, at least about 1 mg/kg, at least about 5 mg/kg, at least about 10 mg/kg, at least about 20 mg/kg, at least about 50 mg/kg, at least about 100 mg/kg, in some embodiments the effective dose is from about 1 to 50 mg/kg. Dosing may be daily, every 2 days, every 3 or more days, e.g., weekly, semi-weekly, bi-weekly, monthly, etc. Dosing may be parenteral, including sustained release formulations. Dosing may be maintained for long periods of time, e.g., months, or years, to maintain desirable glucose and fatty acid levels.

The pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are commercially available. Any compound useful in the methods and compositions of the invention can be provided as a pharmaceutically acceptable base addition salt. “Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

In some embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

A carrier may bear the agents in a variety of ways, including covalent bonding either directly or via a linker group, and non-covalent associations. Suitable covalent-bond carriers include proteins such as albumins, peptides, and polysaccharides such as aminodextran, each of which have multiple sites for the attachment of moieties. The nature of the carrier can be either soluble or insoluble for purposes of the invention.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249:1527, 1990 and Hanes, Advanced Drug Delivery Reviews 28:97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

The following example(s) is/are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Example 1—Prediction and Detection of Capped Peptides in Mouse Plasma

Here we provide evidence to show that capped peptides constitute a large class of previously unstudied mammalian signaling peptides. Capped peptides are endogenously present in mouse and human plasma, where their levels are dynamically regulated by physiologic perturbations. Capped peptides exhibit post-translational N- and C-terminal modifications (pyroglutmylation and amidation, respectively) that resemble that of other peptide hormones and neuropeptides. Functional studies for previously orphan capped peptides uncover functional bioactivity for previously orphan members of this class. The vast majority of the precise capped peptide sequences reported here have not been previously described as chemically defined, endogenous substances in mammals. In fact, our detection of >100 capped peptides in mouse and human plasma suggests that the N- and C-terminal “capping,” rather than being unusual and rare post-translational modifications, instead defines a distinct chemical motif that is present in a large class of peptides with potential to mediate diverse axes of cell-cell communication.

Our ability to endogenously detect capped peptides was enabled by a custom mass spectrometry pipeline that uses a targeted mass spectrometry approach with authentic peptide standards. This method was inspired by classical approaches in targeted metabolomics, where small molecule mass-to-charge ratios and retention times are routinely compared against synthetic standards. The generality and simplicity of this approach was demonstrated by profiling the capped peptides present in blood plasma from two different species. Importantly, such a targeted approach obviates the need for large-scale database searching, thereby overcoming the problem of potential false discovery rate over-correction which may exclude true positive detection events. Because of potential limits of detection, it is not unreasonable to imagine that additional capped peptides that were undetectable here might indeed be endogenously present using more sensitive mass spectrometry methods. Projecting forward, these data also suggest that more targeted mass spectrometry approaches using synthetic standards, particularly on subsets of privileged peptide modifications and/or sequences, might be a powerful approach to identify peptides of specific structure and/or sequence within large, complex mass spectrometry datasets.

Peptide hormones and neuropeptides are fundamental signaling molecules that control diverse aspects of mammalian homeostasis and physiology. Using a hybrid computational-biochemical approach, we demonstrate the endogenous presence of a large and sequence diverse family of orphan blood-borne peptides that we call “capped peptides.” Capped peptides are modified fragments of secreted proteins and present in human and mouse blood. They exhibit structural and regulatory characteristics similar to other signaling peptides, including N-terminal pyroglutamylation, C-terminal amidation, cell type-specific expression, and dynamic environmental and physiologic regulation.

To define potential modified fragments from existing classically secreted proteins, we first used Uniprot (Consortium, 2023) to curate a collection of protein sequences corresponding to classically secreted mouse proteins. Our initial collection of N=2,835 sequences contained many known classically secreted proteins, including apolipoproteins, secreted enzymes, and preprohormones. Next, we sought to define potential fragments of these sequences that might also harbor additional post-translational modifications of the N- and C-termini. For these modifications, we focused on N-terminal pyroglutamylation and C-terminal amidation (FIG. 1A). C-terminal amidation sequences were identified by the presence of a glycine-dibasic GKK/GKR tripeptide (FIG. 1A). N-terminal pyroglutamylation sequences were identified by the presence of a glutamine (Q) 3 to 20 amino acids upstream of the glycine-dibasic motif (FIG. 1A and FIG. 14A).

Our rationale for this set of prediction criteria, and for the focus on peptide fragments with coincident N-pyroglutamyl and C-amidation modifications, was based on the conceptual basis that: (1) these modifications have previously been shown to enhance receptor binding and plasma stability for other peptide hormones, therefore providing higher likelihood that our predicted sequences would be detectable by mass spectrometry and also exhibit bona fide signaling and bioactivity; (2) the glycine dibasic sequence is a well-established C-terminal cleavage motif that, after proteolysis, results a C-terminal glycine which can subsequently be enzymatically modified via PAM to generate the C-terminal amide; (3) biosynthesis of other N-terminal pyroglutamylated signaling peptides often occurs via an N-terminal glutamine, rather than a glutamate. Using this computational framework, we predicted a total of 216 potential cleaved and modified mouse peptides from 186 classically secreted proteins encoded in the mouse genome (FIG. 1B). We call this class of modified peptides “capped peptides” owing to their terminal modifications which function as “caps” of the intervening peptide sequence (FIGS. 1A-1B). Our prediction of potentially hundreds of such modified sequences suggests that chemical capping, rather than being unusual and rare modifications that have been previously reported in only three mammalian peptide hormones (GnRH, TRH, and gastrin), might in fact define a distinct chemical class and potentially functional motif present in a large number of signaling peptides.

To determine whether capped peptides are produced endogenously, we used a targeted mass spectrometry approach to directly measure the levels of all predicted capped peptides in mouse plasma. Our targeted mass spectrometry strategy was inspired by standard approaches in small molecule metabolomics, where analytes are confidently identified by comparison of retention times and mass-to-charge (m/z) ratios to authentic standards that produced via chemical synthesis. Importantly, this mass spectrometry-based workflow obviates the need for identification via database searching that is classically associated with untargeted peptidomics or shotgun proteomics. Towards this end, we first isolated total plasma peptidome from mouse plasma. Briefly, mouse plasma was boiled to inactivate proteases, reduced with DTT, alkylated with iodoacetamide, and concentrated using a C8 column. In parallel, we used solid phase peptide synthesis to generate authentic peptide standards for all 216 predicted capped peptides (FIG. 1C). A mixture of the 216 peptide standards was also reduced, alkylated, and concentrated. Total mouse plasma peptides were then compared to the synthetic peptide mixture by high resolution liquid chromatography-mass spectrometry (LC-MS) on a quadrupole-time-of-flight (qTOF) mass spectrometer.

A representative example of a positive detection event for CAP-TAC1 (pGlu-FFGLM-NH2, FIG. 1D), a capped peptide derived from amino acids 63-68 of full-length TAC1 (protachykinin-1), is shown in FIG. 1E. Here, the authentic CAP-TAC1 standard exhibits identical m/z ratio and retention time to an endogenous plasma peak (m/z=724.3, retention time=28.5 min, FIG. 1E). As further validation of this structural assignment and detection event, MS/MS studies of the authentic CAP-TAC1 standard and the endogenous peak revealed nearly identical fragmentation patterns (FIG. 1F). Manual inspection of the daughter ions revealed that many could be directly assigned to the CAP-TAC1 sequence, including b3 (+1, m/z=406.2), b4 (+1, m/z=463.2), and a5 (+1, m/z=548.3) (FIG. 1F).

Across all predicted capped peptides, 64 of the 216 (30%) exhibited high confidence detection in blood plasma by mass spectrometry (FIG. 1G and FIGS. 14B-14E). The remaining 152 capped peptides (70%) did not exhibit a peak at the retention time of the authentic standard. By comparison to an external standard curve, the detected mouse capped peptides exhibited circulating concentrations in the range of ~0.1-100 nM (FIG. 1H). We conclude that capped peptides are a large family of endogenously circulating molecules. Our inability to detect 152 of the predicted capped peptides may either reflect true absence of post-translational processing to generate those fragments, or alternatively, the endogenous presence of the capped peptide is at a level below our detection limit. Regardless, that many predicted capped peptides could in fact be endogenously detected by a targeted mass spectrometry pipeline also suggest that specific proteolytic processing and capping to produce protected peptide fragments is much more prevalent than previously anticipated.

### Example 2—Sequence and Gene-Level Analysis of Mouse Capped Peptides

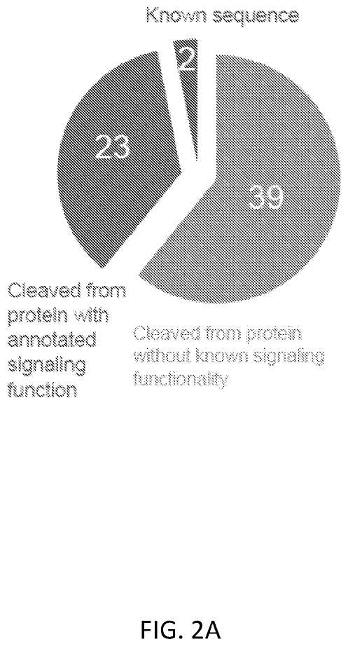

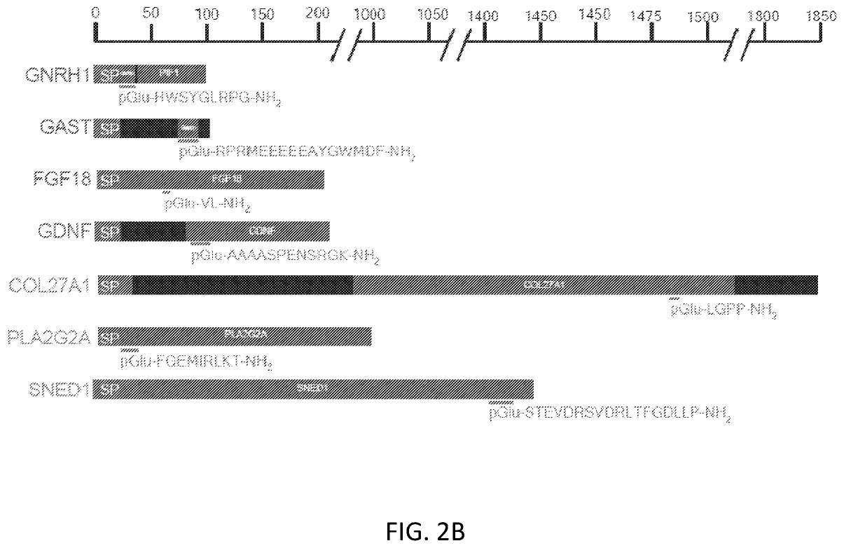

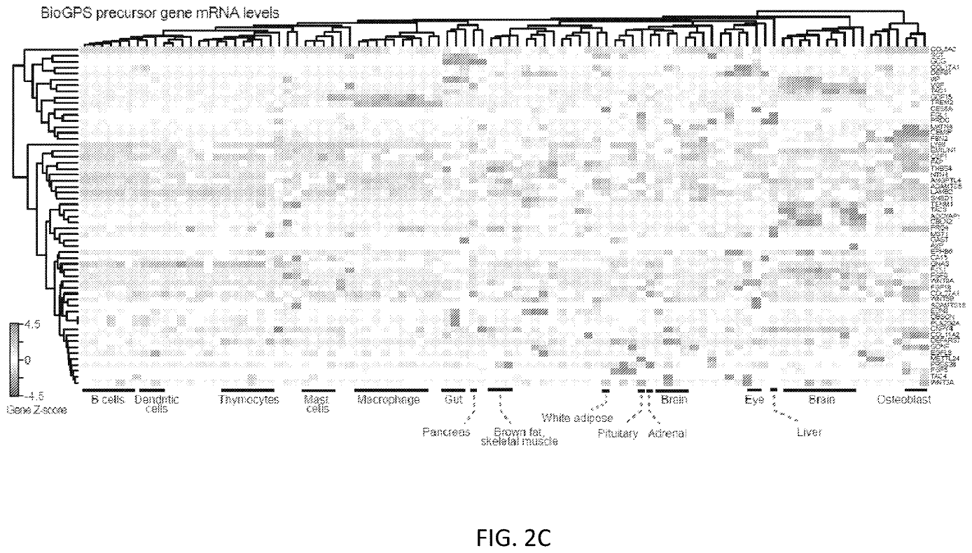

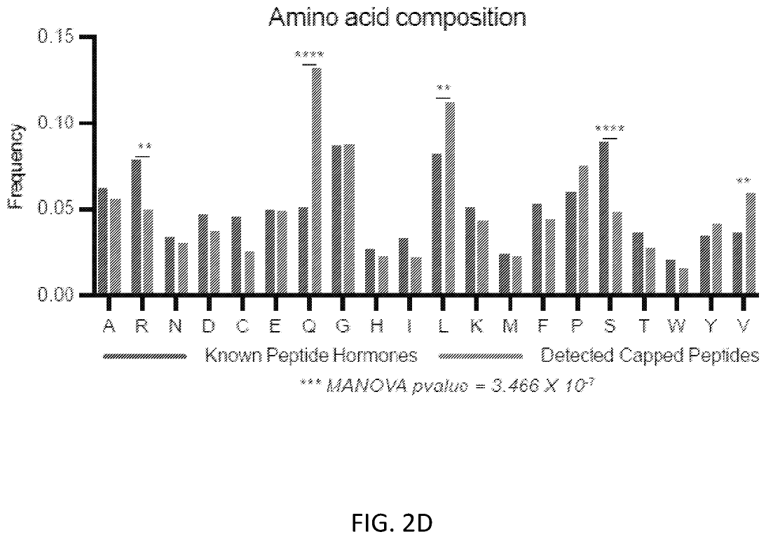

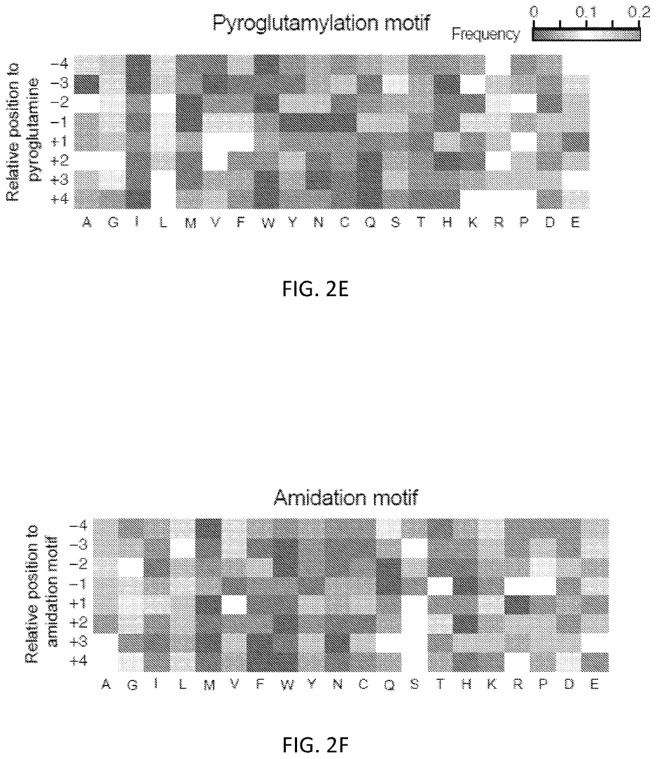

We next examined the sequences and set of full-length preproprecursor proteins of the detected capped peptides. Two of the detected capped peptides, CAP-GNRH1 (pGlu-HWSYGLRPG-NH2) and CAP-GAST (pGlu-RPRMEEEEEAYGWMDF-NH2) directly corresponded with the known hormone sequences for GnRH and gastrin (FIGS. 2A-2B). These data demonstrate that our hybrid computational-analytical approach can “re-discover” two of the known signaling peptides that harbor both N-terminal pyroglutamylation and C-terminal amidation. An additional 23 detected capped peptides mapped to preproprecursor proteins and corresponding genes that had previously been annotated to generate polypeptides with signaling bioactivity, but for which a shorter cleavage fragment had not been previously identified. For instance, we observed a capped tripeptide, CAP-FGF18 (pGlu-VL-NH2), derived from amino acids 66-69 of the prepro-FGF18 (fibroblast growth factor 18) (FIG. 2B). A second capped peptide, CAP-GDNF (pGlu-AAAASPENSRGK-NH2), mapped to amino acids 94-106 of GDNF (glial cell line-derived neurotrophic factor) (FIG. 2B). Classically, FGF18 is a so-called “paracrine” member of the fibroblast growth factor (FGF) family and has diverse roles in the development of several tissues, including bone, lung, and the nervous system (Itoh et al., 2016). GDNF is a major growth factor that promotes the survival of dopaminergic and motor neurons; outside of the nervous system, GDNF is also a morphogen in the kidney and a spermatogonia differentiation factor (Airaksinen and Saarma, 2002). Our detection of these two capped peptides in blood plasma suggests that FGF18 and GDNF might also exhibit endocrine functions via cleavage fragments generated from their canonical polypeptide sequences. Lastly, the remaining capped peptides (39/64, ~61%) mapped to preproprecursors for proteins and genes that had not been previously suggested to have any roles in signaling. These include CAP-COL27A1 (pGlu-LGPP-NH2), which is cleaved from a collagen protein; CAP-PLA2G2A (pGlu-FGEMIRLKT-NH2) which is cleaved from a phospholipase sequence; and CAP-SNED1 (pGlu-STEVDRSVDRLTFGDLLP-NH2) which is derived from the poorly studied protein SNED1 (FIG. 2B).

To further understand the cellular and tissue origin of capped peptides, we next examined the mRNAs of the home genes encoding the capped peptide preproprecursors. We used BioGPS (Wu et al., 2016) as a reference mouse tissue gene expression dataset. As shown in FIG. 2C, this set of mRNAs exhibited both cell type-specific as well as widespread tissue expression. For instance, a strong enrichment of home gene mRNAs for certain capped peptides was found in the brain (e.g., CAP-CBLN2, CAP-TENM1, CAP-TAC3, CAP-ADCYAP1), in bone (e.g., CAP-MATN4, CAP-CEMIP), and in macrophages (e.g., CAP-GDF15, CAP-TREM2). Conversely, mRNAs corresponding to other capped peptides exhibited more diffuse tissue expression across multiple cell types and organs (e.g., CAP-VIP enrichment in both brain and gut and CAP-COL5A2 expression in >10 tissues).

Lastly, we performed more detailed amino acid composition and sequence analysis of the detectable capped peptides from mouse plasma. As a reference and comparison set, we once again used Uniprot to manually curate a set of known mouse peptide hormones and neuropeptides. Glutamine was enriched in capped peptides compared to the reference set of known peptide hormones and neuropeptides, which was expected based on our original computational search criteria. In addition, two hydrophobic amino acids, valine and leucine, were also more prevalent in capped peptides, whereas two polar amino acids (arginine, serine) were less represented (FIG. 2D). To understand whether there might be additional sequence-specific determinants of capping beyond our original N-terminal Q and C-terminal GKK/GKR motifs, we examined the amino acid sequences centered around the N- and C-termini. A modest enrichment of glycine and leucine were observed to flank both the N-terminal pyroglutamylation motif (FIG. 2E). In addition to glycine and leucine enriched around the C-terminal amidation motif, we also observed a strong enrichment for alanine at the +2 position (FIG. 2F). Together, these data demonstrate that capped peptides are produced from diverse tissues and exhibit specific patterns of amino acid composition and sequence.

### Example 3—Dynamic Regulation of Capped Plasma Levels in Mice

Many signaling peptides exhibit dynamic regulation in a manner dependent on internal physiologic state or external environmental conditions. We therefore measured the circulating levels of capped peptides after six distinct perturbations that spanned a wide range of physiologic processes, environmental stimuli, organ systems, and time scales: 16 h fasting vs. fed, 8-weeks high fat diet feeding vs. chow feeding, lipopolysaccharide (LPS, 0.5 mg/kg, intraperitoneal) vs. vehicle, 6 AM vs. 6 PM, acute treadmill running (1 h) vs. sedentary, and 3 months vs. 24 months old. For each comparison, mouse plasma was collected and processed as described previously and capped peptides were quantified by LC-MS (FIG. 1).

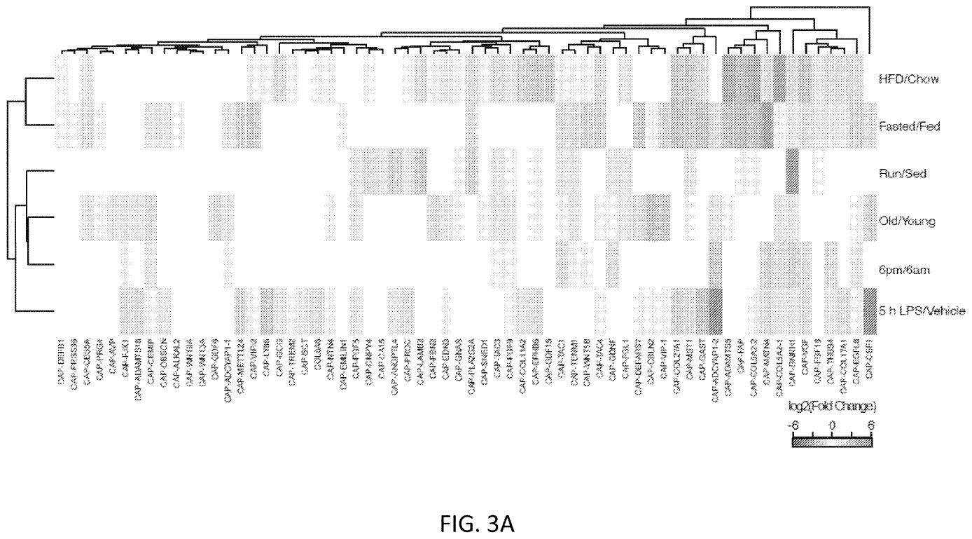

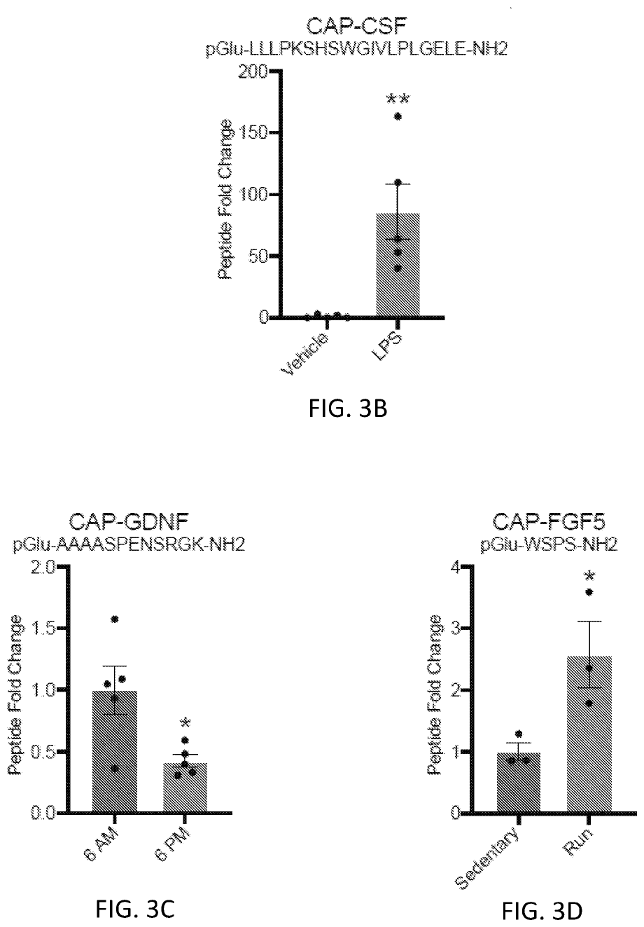

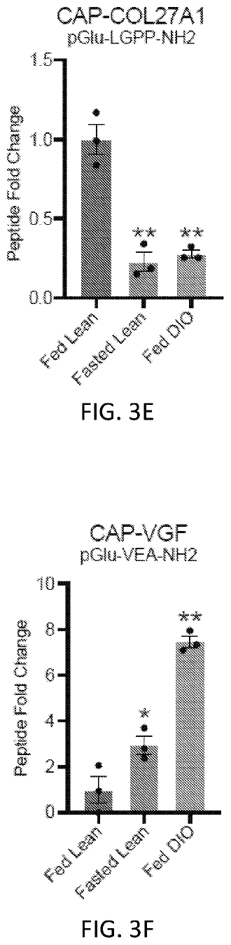

As shown in FIG. 3A, each physiologic comparison resulted in bidirectional regulation of a unique subset of capped peptides. Across all measurements (N=384, corresponding to N=64 capped peptides in each of N=6 conditions), we observed a total of N=93 (24%) instances for a particular capped peptide being elevated (N=39) or suppressed (N=54) by more than 2-fold in any condition. 73% (47/64) of the capped peptides exhibited at least one instance of 2-fold change across any condition; similarly, 100% of the conditions (6/6) yielded at least one capped peptide that was changed by more than 2-fold.

The capped peptide/perturbation pair resulting in the most dramatic regulation was CAP-CSF1 (pGlu-LLLPKSHSWGIVLPLGELE-NH2), derived from amino acids 419-438 of full-length prepro-CSF1. Plasma CAP-CSF1 levels were induced by ~84-fold after LPS treatment (P<0.01, FIGS. 3A-3B). Importantly, CAP-CSF1 levels were unchanged in any of the other comparisons (FIG. 3A), establishing that induction of CAP-CSF1 in plasma is a specific response to an inflammatory stimulus. Previously, the most well-known polypeptide product derived from full-length prepro-CSF1 is m-CSF1 (macrophage colony-stimulating factor 1), which is itself an LPS-inducible cytokine (Benmerzoug et al., 2018). The co-induction of CAP-CSF1 may therefore represent additional, LPS-inducible proteolytic processing of m-CSF1. In addition, we could also identify several other interesting examples of individually regulated dynamic peptides in each of the conditions. For instance, CAP-GDNF was selectively downregulated in plasma collected at 6 PM versus 6 AM (pGlu-AAAASPENSRGK-NH2, 58% reduction, P<0.05, FIG. 3C) and CAP-FGF5 was selectively induced by a single bout of treadmill running (1 h) versus sedentary mice (pGlu-WSPS-NH2, 2.6-fold increase, P<0.05, FIG. 3D).

Beyond high magnitude changes in individual capped peptides in each condition, we also identified examples of capped peptides that exhibited coordinate regulation across multiple physiologic states. For instance, we observed a cluster capped peptides that were coordinately regulated in two distinct nutritional stressors, fasting and high fat diet feeding. Individual examples of dynamic capped peptides within this nutrition-regulated cluster included CAP-COL27A1 (pGlu-LGPP-NH2, ~75% reduction, P<0.05, FIG. 3E) and CAP-VGF (pGlu-VEA-NH2, >3-fold induction, P<0.05, FIG. 3F). The nutritional regulation of this subset of capped peptides, and of CAP-COL27A1 and CAP-VGF in particular, might point to specific functions in nutrient harvesting, fuel metabolism, or energy homeostasis. Together, we conclude that capped peptide levels in the circulation are dynamically regulated in a manner dependent on the specific capped peptide and specific physiologic perturbation.

### Example 4—CAP-TAC1 is a Novel Tachykinin with Homology to Substance P

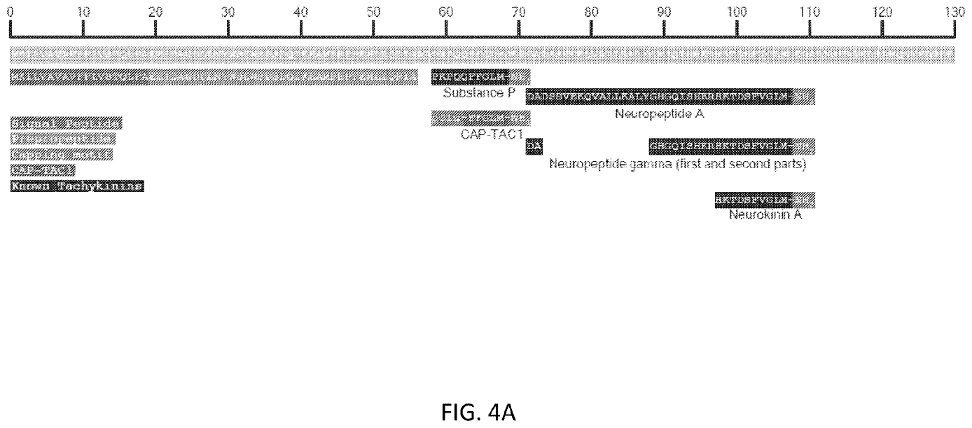

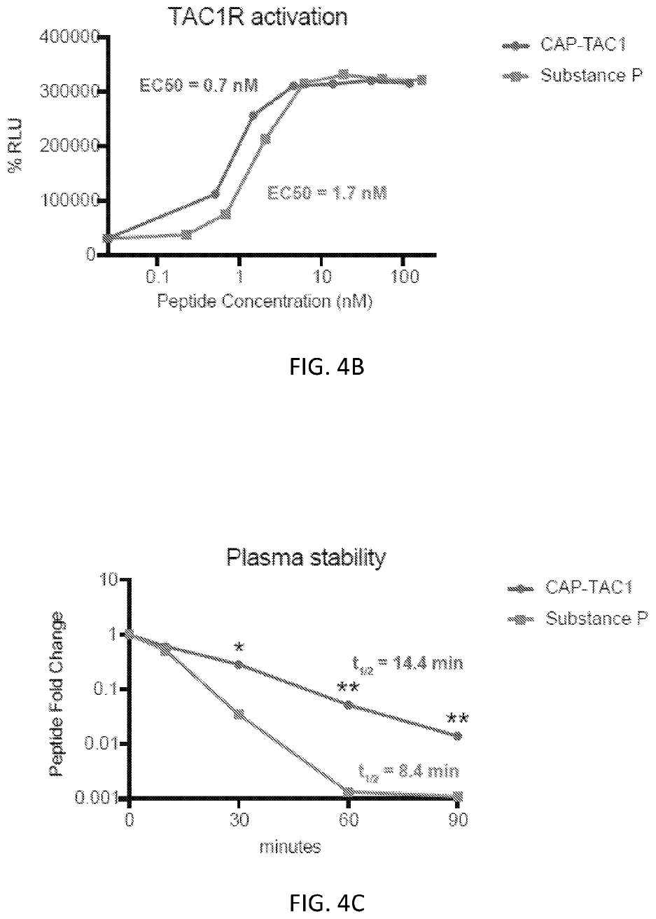

Our data so far suggests that capped peptides exhibit many structural and regulatory features of other well-established peptide hormones and neuropeptides. We next sought to determine whether any of the detectable capped peptides exhibited signaling and/or functional bioactivity. We first focused on CAP-TAC1 (pGlu-FFGLM-NH2), an orphan peptide of previously unknown function. As we already showed in FIG. 1, CAP-TAC1 is robustly detected in blood plasma. The full-length TAC1 preproprotein encodes multiple members of the tachykinin neuropeptides, including Neurokinin A/Substance K, Neuropeptide K/Neurokinin K, Neuropeptide gamma, and Substance P (FIG. 4A) (Steinhoff et al., 2014). Of these known tachykinin neuropeptides, CAP-TAC1 exhibits most homology to substance P.

We noted that the sequence of CAP-TAC1 contains the key consensus C-terminal FXGLM-NH2 motif which is characteristic of all known tachykinin neuropeptides (FIG. 4A). In addition, the C-terminal methionyl amide has been shown to be critical for agonist activity (Escher et al., 1982; Patacchini et al., 1993). The presence of the tachykinin motif and an intact methionyl amide in CAP-TAC1 directly suggested that this capped peptide might also be a tachykinin neuropeptide-like molecule. We therefore used a cellular human TACR1-beta arrestin recruitment assay with a fluorescence readout to directly determine the ability of CAP-TAC1 to agonize the TACR1, a high affinity receptor for substance P (Bhatia et al., 1998). As shown in FIG. 4B, CAP-TAC1 exhibited dose-dependent and high potency agonism of TACR1 (ED50=0.7 nM). As a positive control, substance P exhibited a similar dose-dependent activation (ED50=1.7 nM). Both CAP-TAC1 and substance P exhibited similar levels of maximal activation (CAP-TAC1, 96.6% of maximal response; substance P, 99.8% of maximal response). We conclude that CAP-TAC1 is a full agonist of the TACR1 receptor with potency similar to the previously known TACR1 ligand substance P.

Despite similar agonist activity, CAP-TAC1 and substance P exhibit differences in chemical structure: CAP-TAC1 contains an additional N-terminal pyroglutamylation and is also shorter in length compared to substance P. We reasoned these two chemical differences might result in important functional differences in terms of stability and resistance to proteolytic degradation. To directly test this possibility, CAP-TAC1 (10 UM) and substance P (10 UM) were individually incubated with mouse plasma and incubated at 37° C. and their levels over time were measured by LC-MS. Substance P exhibited time-dependent degradation with a t_{1/2}=8.4 min. By contrast, the rate of CAP-TAC1 degradation was substantially slower (t_{1/2}=14.4 min) (FIG. 4C). In fact, levels of CAP-TAC1 were still detectable after 90 min, a time point when substance P was undetectable (FIG. 4C). Together, these data demonstrate that CAP-TAC1 exhibits similarities (e.g., TACR1 agonism) as well as important differences (e.g., plasma stability) compared to previously described tachykinin neuropeptides.

Our identification of CAP-TAC1, and then demonstration of this molecule as a high affinity agonist of mammalian tachykinin receptors, shows that additional fragments of full-length tachykinin preproproteins may be important endogenous mediators tachykinin signaling.

### Example 5—a 12-Mer Hypophagic Capped Peptide from the Preprocursor Region of GDF15

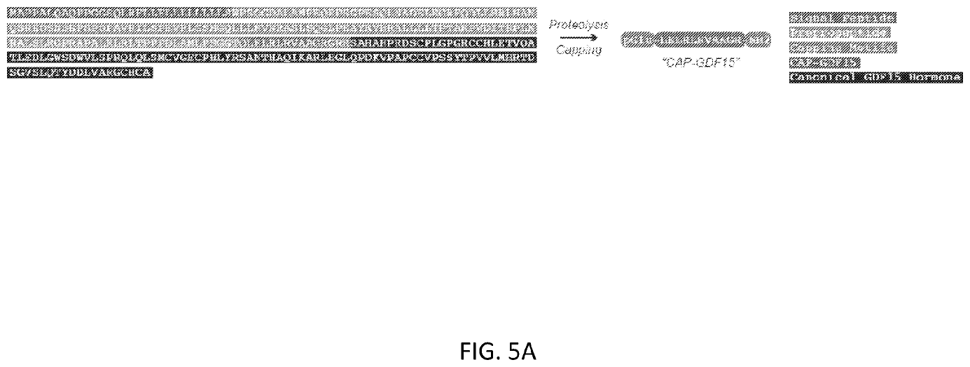

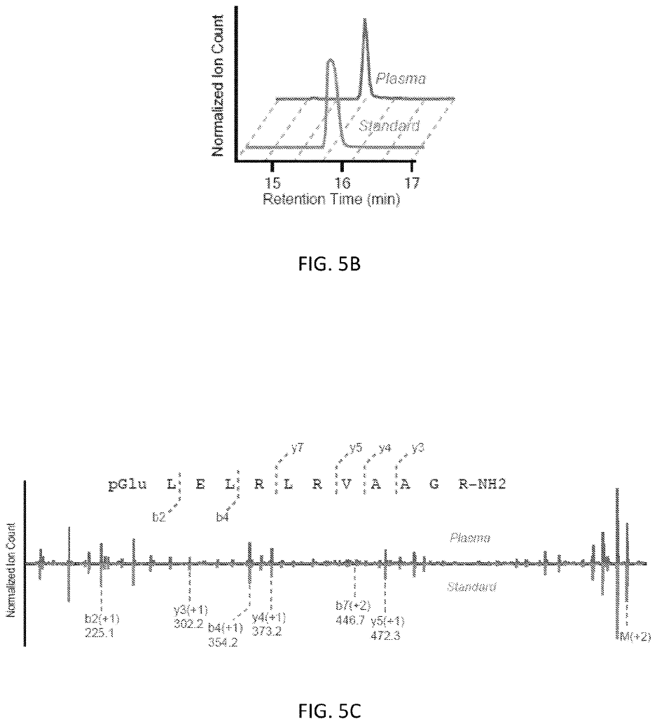

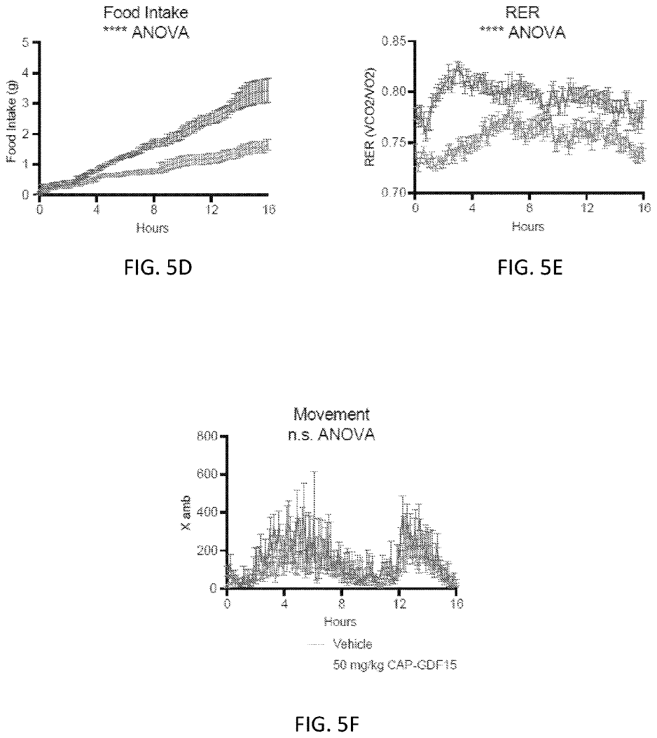

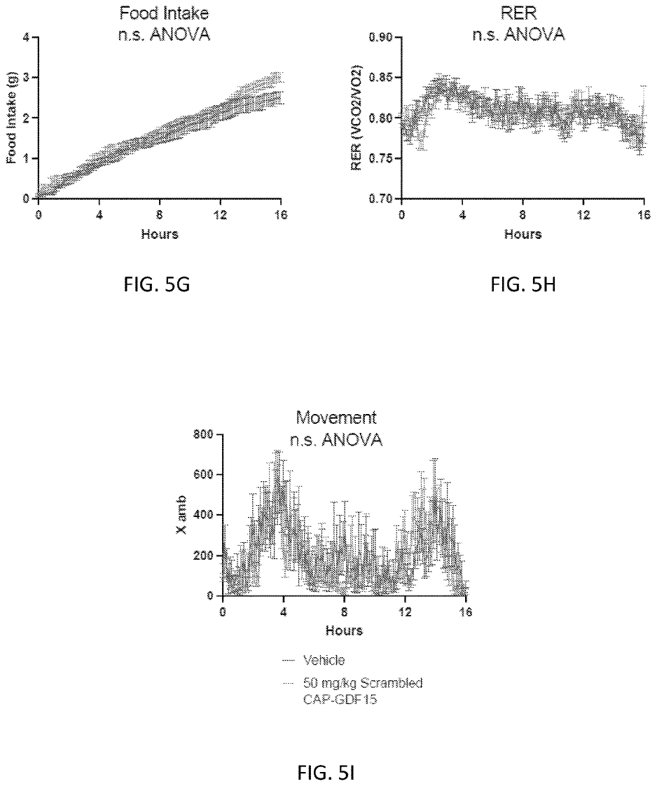

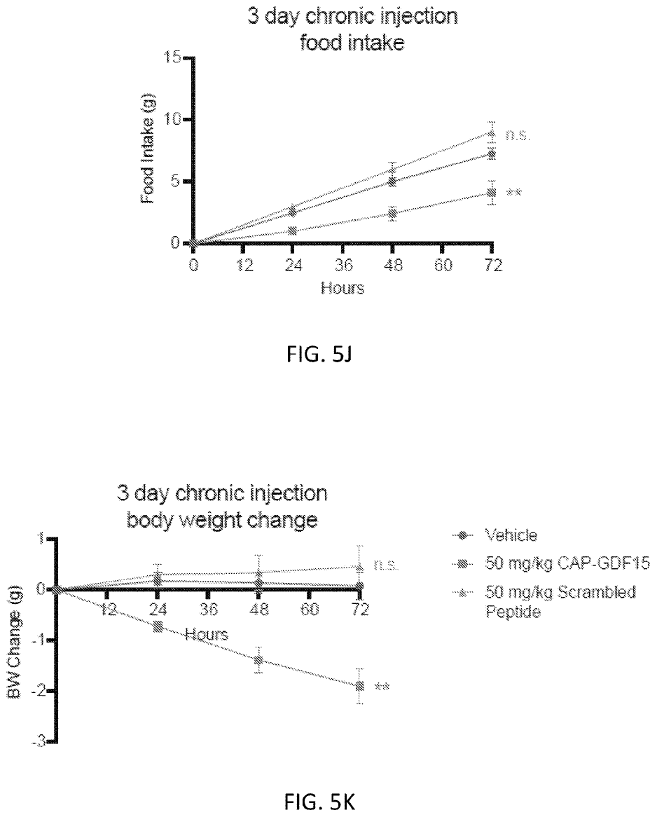

We next sought to understand whether signaling and bioactivity might be indeed a general feature of many capped peptides beyond CAP-TAC1 alone. We next focused on functional studies of a second orphan capped peptide, CAP-GDF15 (pGlu-LELALAVAAGR-NH2, FIG. 5A). Full-length GDF15 is a secreted, 303 amino acid preproprecursor that, upon cleavage at R188, produces a C-terminal 114-amino acid anorexigenic protein hormone which is also called GDF15 (Chrysovergis et al., 2014; Johnen et al., 2007; Macia et al., 2012). Interestingly, CAP-GDF15 mapped to amino acids 174-185, a region just upstream of the canonical GDF15 hormone and localized in the GDF15 prepropeptide region (FIG. 5A). CAP-GDF15 showed identical co-elution with an authentic standard (FIG. 5B). In addition, tandem mass spectrometry fragmentation also revealed a similar fragmentation pattern between the endogenous peak and the authentic standard (FIG. 5C). These data demonstrate that full-length GDF15 precursor encodes at least two polypeptide products.

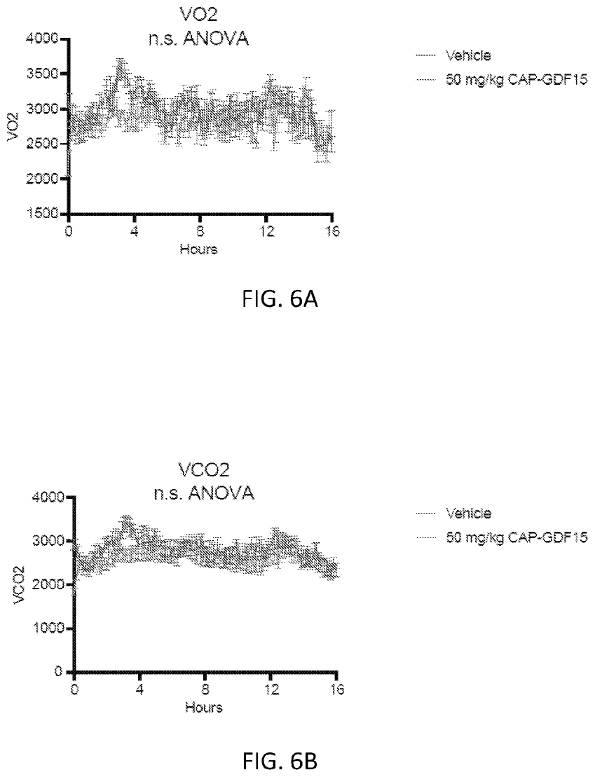

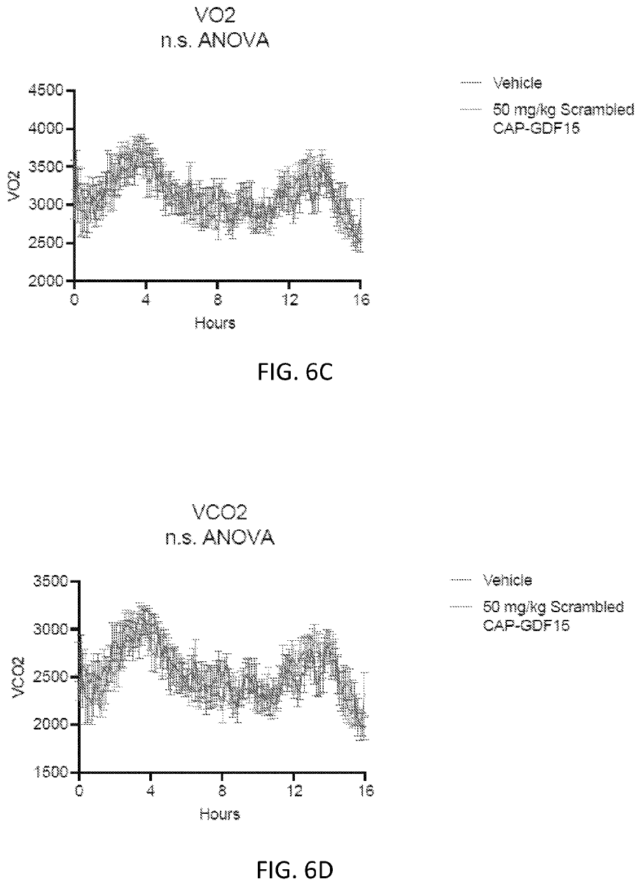

Unlike CAP-TAC1, the amino acid sequence of CAP-GDF15 did not immediately provide insights into its potential functions. However, we reasoned that the hypophagic effects previously demonstrated by overexpression of full-length GDF15 might extend beyond the classical GDF15 hormone alone to also include CAP-GDF15. To test this possibility, we administered a single dose of CAP-GDF15 (50 mg/kg, intraperitoneally) to diet-induced obese mice and measured whole body parameters of energy balance in metabolic chambers. CAP-GDF15 strongly suppressed food intake by ~60% compared to vehicle-treated mice (FIG. 5D). A corresponding and expected suppression of respiratory exchange ratio (RER) was also observed (FIG. 5E). Notably, CAP-GDF15 did not alter movement (FIG. 5F), oxygen consumption (FIG. 6A), or carbon dioxide production (FIG. 6B), demonstrating that the pharmacological effects of this peptide are specific to feeding control rather than other pathways of energy expenditure.

We next synthesized a control CAP-GDF15 peptide that preserved amino acid composition but scrambled the intervening amino acid sequence (scrambled CAP-GDF15, pGlu-GLEALRARLRV-NH2). This scrambled peptide control was completely ineffective in suppressing food intake and RER in metabolic chambers under identical experimental conditions (FIGS. 5G-51 and FIGS. 6C-6D). We conclude that the hypophagic effects of CAP-GDF15 are specific to this amino acid sequence.

Lastly, to determine whether the acute hypophagic effects of CAP-GDF15 would lead to long-term suppression of feeding and obesity, we administered CAP-GDF15 or scrambled CAP-GDF15 (50 mg/kg/day, IP), or vehicle control to diet-induced obese mice. Food intake and body weight were monitored over a three-day period. A durable suppression of food intake in CAP-GDF15-treated mice was observed over the three-day experiment (FIG. 5J). Consequently, and as expected, an increasing reduction in body weight was also detected (FIG. 5K). Importantly, mice treated with the scrambled CAP-GDF15 peptide were indistinguishable in body weight or food intake from control mice (FIGS. 5J-5K). These data show that chronic CAP-GDF15 administration suppresses food intake and reduces body weight in a sequence-dependent manner.

Together with CAP-TAC1, these data on CAP-GDF15 provide functional evidence for the signaling and bioactivity of orphan capped peptides in both cell and animal models. Like CAP-TAC1, the detection of CAP-GDF15 also demonstrates that a single full-length preproprecursor (in this case, full-length GDF15) can generate more than a single bioactive polypeptide product. The observation that CAP-GDF15 is an anorexigenic peptide like the canonical GDF15 hormone raises new questions about the relative physiologic contribution of each CAP-GDF15 and canonical GDF15. In addition, because the sequences are largely distinct, we suspect that the downstream receptor(s) of CAP-GDF15 are likely to be distinct from that of the canonical GDF15 hormone.

### Example 6—Detection of Human Capped Peptides and Sequence Comparison to Mice

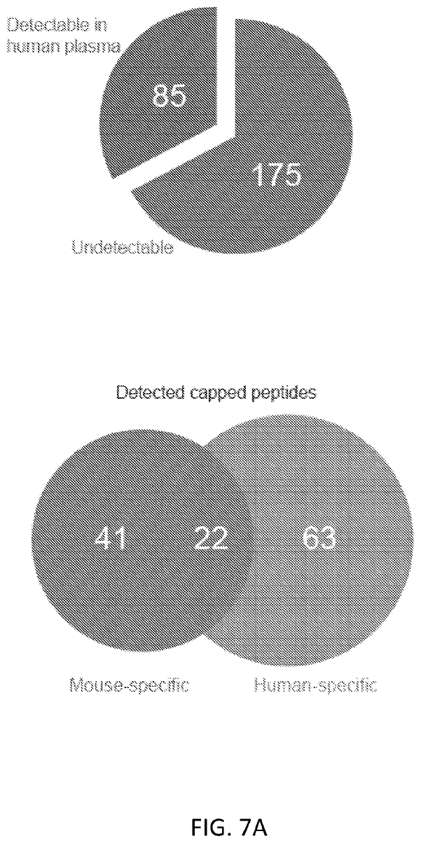

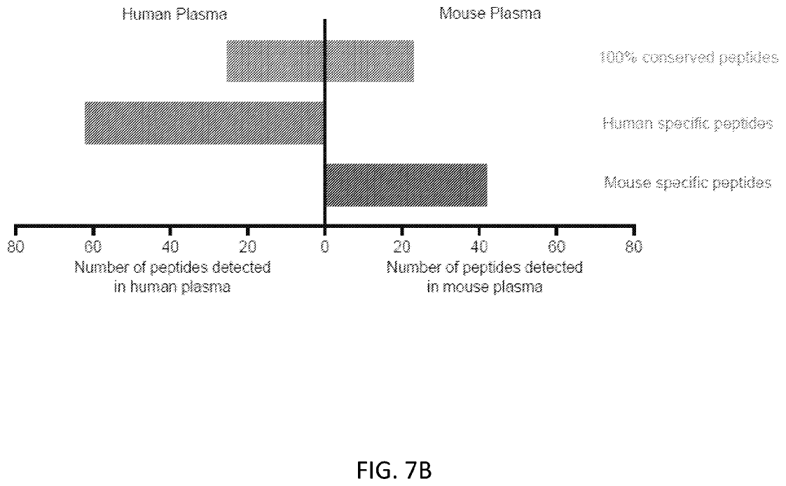

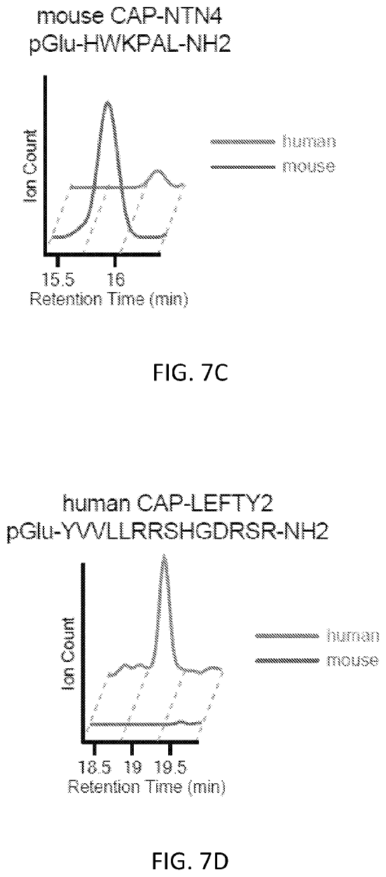

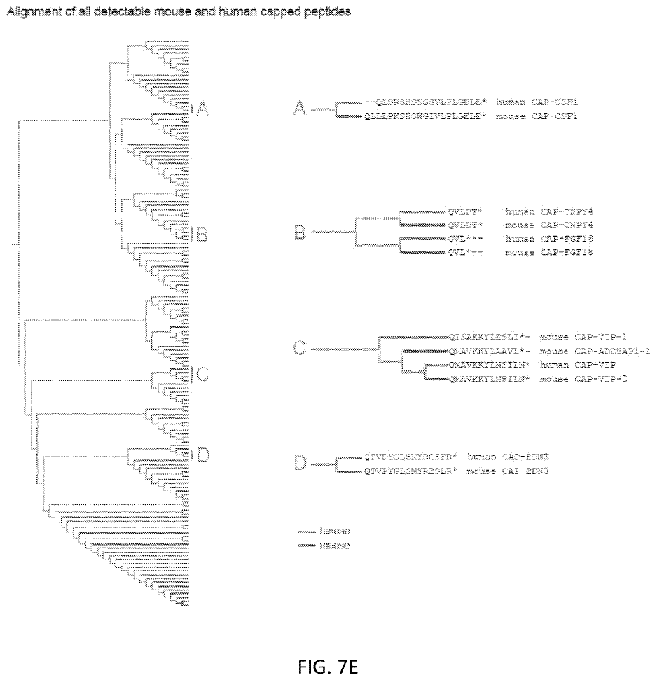

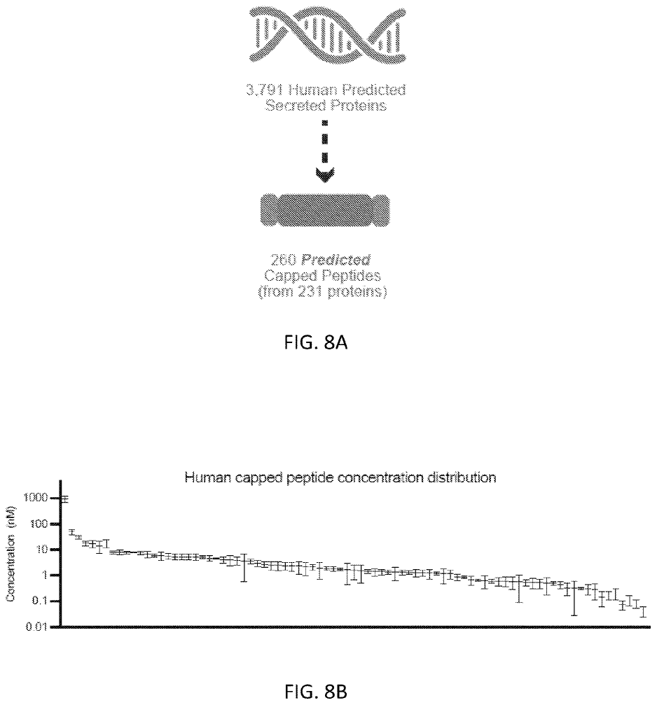

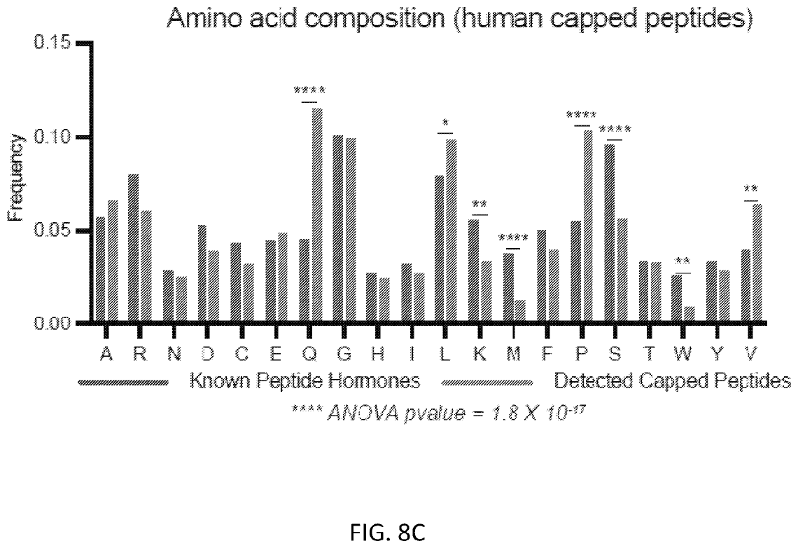

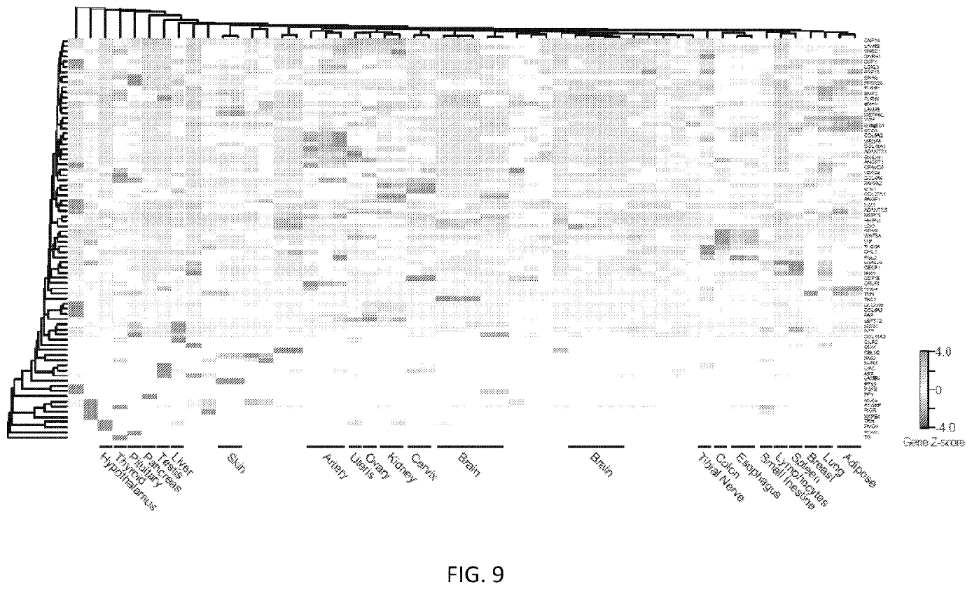

The capped peptide discovery pipeline described here only requires a full genome sequence and authentic peptide standards. Therefore, such an approach should also be readily amenable for discovering capped peptides in other species. Towards this end, we used the same hybrid computational-biochemical workflow as shown in FIG. 1, but now applied to protein sequences corresponding to classically secreted human proteins. Starting from N=3,791 secreted proteins, we predicted a total of 260 potential human capped peptides from 231 proteins (FIG. 15 and FIG. 8A). We synthesized authentic peptide standards by solid phase peptide synthesis corresponding to all 260 possible human capped peptides. Once again, the retention time and parent masses of all authentic capped peptides were compared to endogenous peaks from commercially available pooled human plasma. In total, we robustly detected N=85 human capped peptides by mass spectrometry (FIG. 7A), a number that, by percentage, is similar to that previously observed with mice (30% detected/predicted for mouse, and 33% detected/predicted for human). Human capped peptides exhibited a similar distribution in plasma abundance (as quantitated using an external standard curve, FIG. 8B) and similar sequence characteristics in terms of amino acid composition (FIG. 8C) as mouse capped peptides. Using GTEx as a reference gene expression dataset, human capped peptides were also derived from preproprecursors whose mRNA levels also exhibited tissue-restricted, as well as more broad expression (FIG. 9).

Because of sequence differences between the mouse and human proteome, we predicted that the set of humans capped peptides should be overlapping, but still distinct compared to those present in mouse plasma. Indeed, we detected many capped peptides in both human and mouse plasma that were 100% sequence conserved. In addition, none of the 61 human-specific peptides were detected in mouse plasma, and none of the 41 mouse-specific peptides were found in human plasma (FIG. 7B). Representative extracted ion chromatograms corresponding to conserved and species-specific capped peptides are shown in FIGS. 7C-7D.

Next, we performed a multiple sequence alignment to globally understand the sequence relationship and homology across all capped peptide sequences from both mice and humans. We also performed this analysis to understand whether the human- and mouse-specific capped peptide constituted entirely distinct sequences, high homologous sequences, or some combination of these two possibilities. The resulting dendrogram is shown in FIG. 7E. We selected several sub-clusters as illustrative examples here. Clusters “A” consists of a pair of capped peptides, mouse and human CAP-CSF1. These two are highly homologous sequences derived from amino acids 419-438 and 424-441 of full-length mouse and human CSF1, respectively. However, because the sequences are not 100% identical, mouse and human CAP-CSF1 are considered species-specific in our analysis. A similar example is shown in Cluster “D” where mouse and human CAP-EDN3 differ only by two amino acid residues and are once again considered species-specific, homologous sequences. Clusters “A” and “D” demonstrate that at least a subset of the species-specific sequences is due to differences in amino acid sequences of the corresponding full-length preproproteins from which the capped peptides are derived.

In another example, cluster “B” contained four short 3- and 5-mer capped peptides, which were amongst the shortest sequences in the entire dataset. The 3-mer capped peptides (pGlu-VL-NH2) were derived from the full-length mouse and human FGF18 sequences and exhibited identity between the two species. The other two capped peptides, derived from CNPY4 and again identical between mouse and human, constitute CAP-FGF18 homologs with an aspartyl-threonyl C-terminal extension (pGlu-VLDT-NH2). This cluster demonstrates that highly homologous capped peptides can also be produced from distinct full-length preproprotein precursors.

Lastly, the cluster labeled “C” contained four peptides, three of which were derived from the full-length mouse or human VIP preproprecursor. The three VIP-derived capped peptides correspond with C-terminal fragments of the known PHI-27 and VIP peptide hormones. We also identified a non-VIP-derived peptide, CAP-ADCYAP1, which also exhibited high sequence alignment within this CAP-VIP-enriched cluster. CAP-ADCYAP1 is a C-terminal fragment of the neuropeptide PACAP (pituitary adenylate cyclase-activating polypeptide). These data suggest that the similar signal transduction pathways of VIP and PACAP peptides might also extend to additional fragments of these canonical sequences. We conclude that at least a subset of the human- and mouse-specific capped peptides represent highly homologous sequences. These data also globally identify similarities as well as important differences in the sequences of capped peptides between two species.

### Example 7—CAP-TAC1 Induces Body Weight Loss Independent of Food Intake

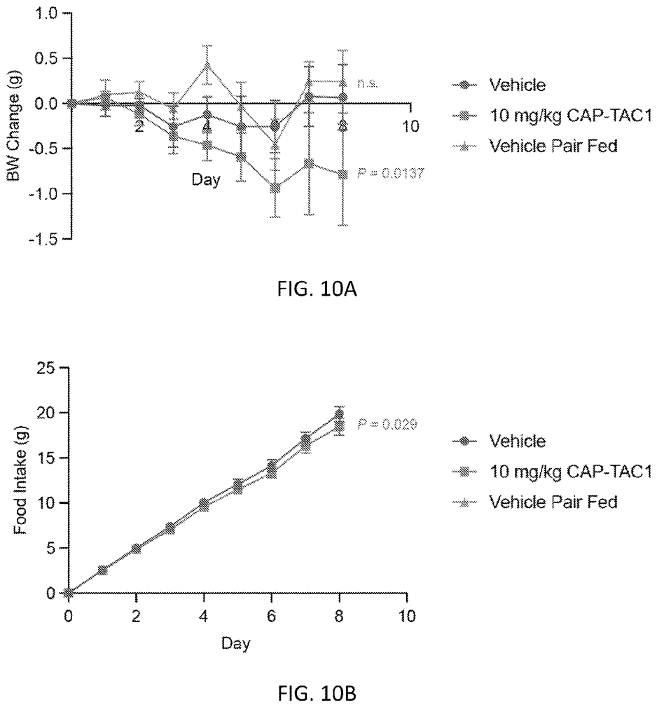

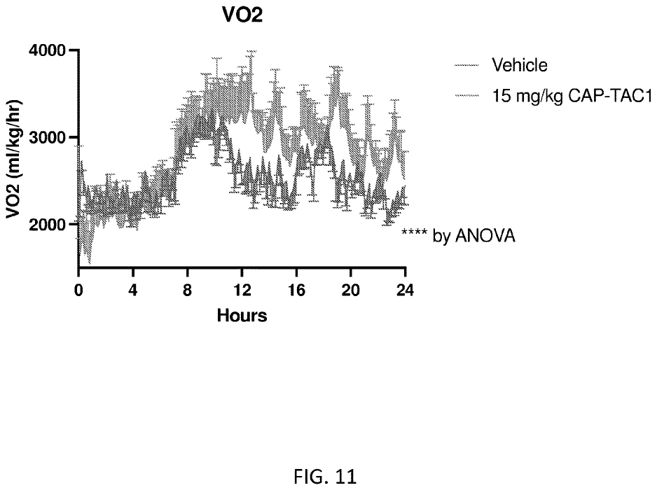

CAP-TAC1 induces body weight loss in diet induced obese mice (FIG. 10A), independent of any changes of food intake (FIG. 10B), suggesting CAP-TAC1 chronically activates energy expenditure. CAP-TAC1 induces an increase in oxygen consumption with a single injection in diet induced obese mice (FIG. 11), showing CAP-TAC1 acutely activates energy expenditure.

### Example 8—Human CAP-GDF15 Inhibits Food Intake

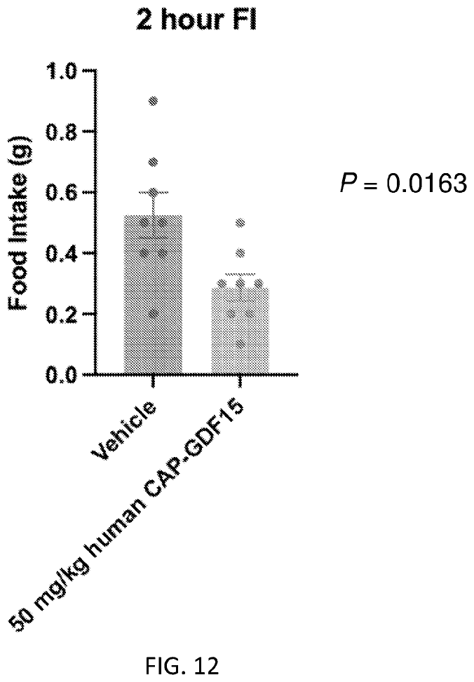

The human homologue of CAP-GDF15 acutely inhibits food intake in diet induced obese mice (FIG. 12), showing the human homologue has a similar hypophagic effect in mice as the endogenous mouse CAP-GDF15, despite some sequence differences.

### Example 9—CAP-WNT9A Shows Growth Hormone Secretagogue Activity

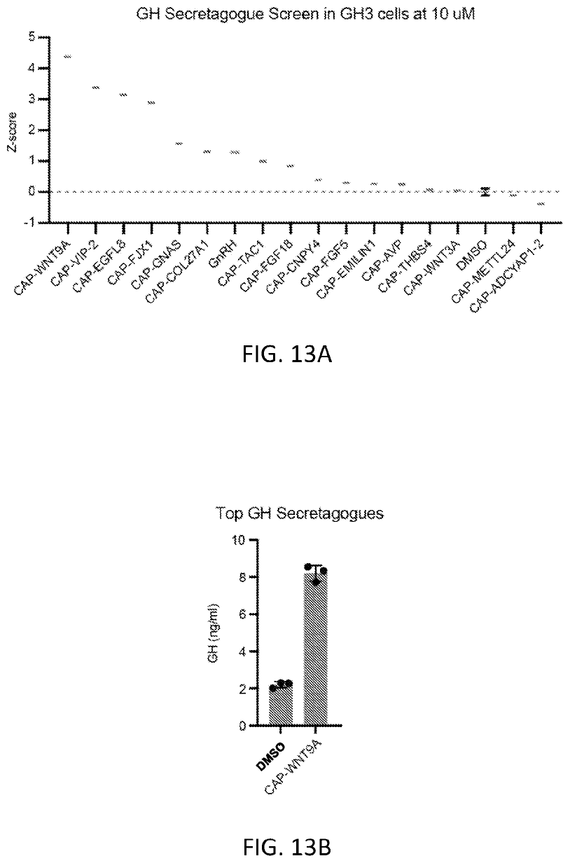

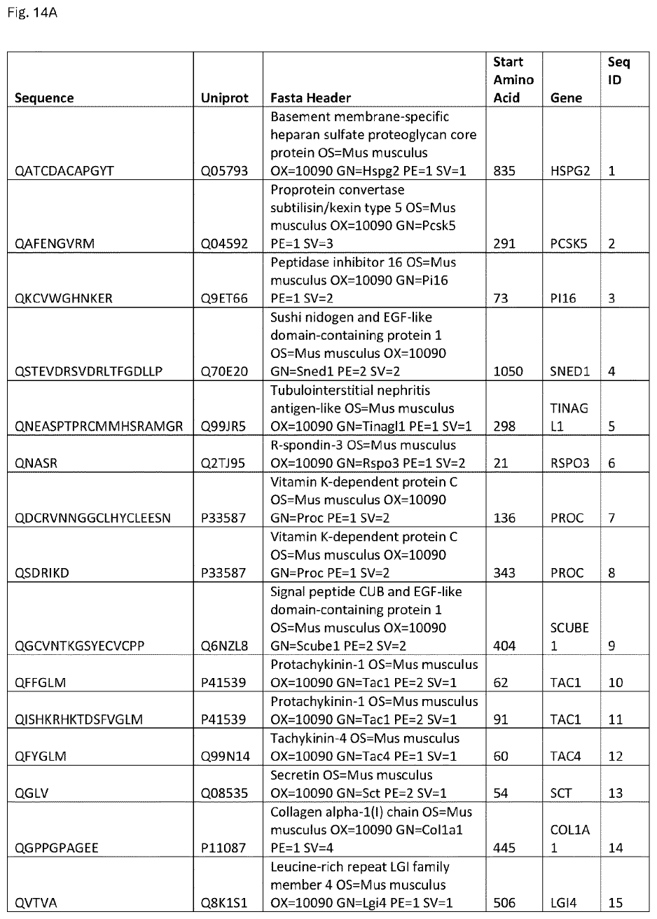

In a functional screen in GH3 cells, a rat pituitary cell line, various capped peptides are shown to activate growth hormone secretion (FIG. 13A). Of the capped peptides that were screened, CAP-WNT9A shows the highest growth hormone secretagogue activity. After a one hour treatment of CAP-WNT9A, growth hormone concentration in media is increased by four times (FIG. 13B).

### Materials and Methods

Mice and treatments. Animal experiments were performed according to a procedure approved by the Stanford University Administrative Panel on Laboratory Animal Care. Mice were maintained in 12-h light-dark cycles at 22° C. and about 50% relative humidity and fed a standard irradiated rodent diet. Where indicated, a high-fat diet (D12492, Research Diets 60% kcal from fat) was used. Male C57BL/6J (stock number 000664) and male C57BL/6J DIO mice were purchased from the Jackson Laboratory (stock number 380050). For studies in high fat diet-fed mice, peptides were dissolved in 18:1:1 (by volume) of saline: Kolliphor EL (Sigma Aldrich): DMSO and administered to mice by intraperitoneal injections at a volume of 10 μl/g at the indicated doses for the indicated times. For lipopolysaccharide injection, LPS was dissolved in saline and administered to mice at a volume of 5 μl/g at indicated dose. For fasting, food was removed from mice for 16 h. For running, a six-lane Columbus Instruments animal treadmill (product 1055-SRM-D65) was used with following 1 h protocol: 10 min at 6 m/min, 50 min at 18 m/min, and increase every 2 min by 2 m/min for the last 10 minutes, all at 12° incline. For all treatment experiments, mice were mock injected with the vehicle for 3-5 days until body weights were stabilized. Heparin plasm was harvested by submandibular bleed. For all experiments, mice were randomly assigned to treatment groups. Experimenters were not blinded to groups.

CAP-TAC1 treatments in mice. Injected diet induced obese (on high fat diet, 4 months old, male, C57BL/6J, Jackson cat #380050) mice daily with vehicle (18:1:1 Saline: DMSO: Kolliphor EL) or 10 mg/kg CAP-TAC1 (pGlu-FFGLM-NH2). The pair fed group was started one day later and fed the same food the CAP-TAC1 mice ate daily. Mice were singly housed and injected daily ~12 μm. Mouse body weights and food weights were measured at the same time. (N=6 for vehicle, N=7 for CAP-TAC1 and pair fed groups.)

Human CAP-GDF15 treatments in mice. Singly housed diet induced obese (on high fat diet, 4 months old, male, C57BL/6J, Jackson cat #380050) mice were injected with vehicle (18:1:1 Saline: DMSO: Kolliphor EL) or 50 mg/kg CAP-GDF15 human homologue (pGlu-LELHLRPQAAR-NH2) once at ~5 μm. Food weight was measured before injection and 2 hours post to calculated food intake. (N=8 mice/group)

In vitro studies. GH3 cells (rat pituitary cell line, ATCC cat. #CCL-82.1) were cultured in F-12K medium with 2.5% fetal bovine serum and 15% horse serum. GH3 cells were washed, resuspended in Krebs Ringer Hepes Buffer, diluted to 0.5 million cells/mL, and aliquoted into tubes (300 μL/sample). The GH3 cells were then starved for 60 minutes in Krebs Ringer Hepes Buffer. The capped peptides (N=1 in screen and N=3 in repeat with CAP-WNT9A) or DMSO (N=3) control were added at a 10 μM final concentration. GH3 cells were incubated for 1 hour. Cells were pelleted with 5 min centrifuge spin at 500×g at 4 C. Grown hormone was measured in the supernatant with an ELISA kit (Thermo Cat #KRC5311) according to product protocol. Z-score of growth hormone concentration is shown. CAP-WNT9A (pGlu-WGGCGDNLKYSSKFVEFL-NH2) (the top hit from the screen) was validated with the same protocol in triplicate (N=3).

Uniprot dataset curation. Lists of classically secreted proteins were obtained from Uniprot using the keyword “secreted” and filtering for either human or mouse species. Known peptide hormone sequences were obtained from Uniprot by first filtering for proteins annotated with keyword “hormone” for function and subsequently extracting out the specific hormone sequences from the peptides listed under the PTM annotations.

Computational prediction of capped peptides. Capped peptide prediction was accomplished using an in-house custom algorithm written in python. First, a list of classically secreted proteins was obtained from Uniprot using the keyword “secreted.” Next, C-terminal amidation motifs were identified based on a GKR or GKK sequence indicative of dibasic cleavage and then amidation. N-terminal pyroglutamylation was identified by searching for Q residues within 20 amino acids upstream of the amidation motif, and capped peptides were predicted to be the inclusive sequence between the N-terminal (pyro) glutamine and the C-terminal amidation.

Solid-phase synthesis of capped peptides. Capped peptides were synthesized using standard solid phase synthesis protocols.

Plasma and authentic peptide standards preparation for peptidomics. To carry out plasma peptidomics, 1 μl of protease inhibitor (HALT) was added to 100 μl of plasma. Plasma was diluted to 1:6 plasma: Tris-HCl buffer (100 mM Tris-HCl, pH 8.2) and boiled at 95° C. for 10 minutes. In total, 1 ml of pooled plasma was used per replicate. 1 mM dithiothreitol (DTT) was added, samples were vortexed and incubated for 50 minutes at 60° C. Iodoacetamide was added to obtain a final concentration of 5 mM and incubated at room temperature for 1 hour in the dark. Formic acid was added to 0.2% final concentration. Samples were centrifuged at 15,000 rpm for 20 min. Supernatants were concentrated with C8 columns (Waters, WAT054965), washed/desalted with water, and eluted in 100 μl of 80% acetonitrile. Samples were centrifuged at 15,000 rpm for 10 min. Supernatant was collected for liquid chromatography-mass spectrometry (LC-MS) analysis. All authentic peptide standards were pooled into 1 ml of Tris-HCl buffer (100 mM Tris-HCl, pH 8.2) and prepared in the same way as described above.

LC-MS detection of capped peptides. LC-MS was performed on an Agilent 6520 Quadrupole time-of-flight LC-MS instrument. MS analysis was performed using electrospray ionization (ESI) in positive mode. The dual ESI source parameters were set as follows: the gas temperature at 325° C., the drying gas flow rate at 13 l/min, the nebulizer pressure at 30 psig, the capillary voltage at 4,000 V, and the fragmentor voltage at 175 V. Separation of peptides was conducted using a C18 column with reverse phase chromatography. Mobile phases were as follows: buffer A, 100% water with 0.1% formic acid; buffer B, 90:10 acetonitrile:water with 0.1% formic acid. The LC gradient started at 95% A with a flow rate of 0.7 ml/min from 0 to 3 minutes. The gradient was then linearly increased to 40% A/60% B from 3 to 28 minutes and subsequently flushed at 5% A/95% B for 4 minutes and equilibrated back to 95% A/5% B for 6 minutes all at a flow rate of 0.7 ml/min. A positive capped peptide detection required a peak of exact mass (±50 ppm) with total area >1000 ion count and co-elution with the corresponding authentic synthetic standard. Exact masses and retention times of all detected peptides were recorded.

Targeted LC-MS/MS. Targeted LC-MS/MS spectra were obtained using Agilent 6545 Quadrupole time-of-flight LC-MS instrument. The dual ESI source parameters were set as follows: the gas temperature at 325° C., the drying gas flow rate at 13 l/min, the nebulizer pressure at 30 psig, the capillary voltage at 4,000 V, the fragmentor voltage at 185 V, the sheath gas temperature at 350° C., and the sheath gas flow rate at 11 l/min. The LC separation was done as described above. For CAP-GDF15, targeted LC-MS/MS method fragmented the +2 ion (m/z=682.4) over the entire run with a collision energy of 40 and a narrow isolation width (~1.3 m/z). For CAP-TAC1, targeted LC-MS/MS method fragmented the +1 ion (m/z=724.3) over the entire run with a collision energy of 25 and a narrow isolation width (~1.3 m/z).

TACR agonist assay. Dose-response curves for CAP-TAC1 and positive control Substance P on the agonism of TACR1 was measured by a Eurofins Discovery using human TACR1-transfected PathHunter beta-arrestin CHO-K1 cells.

Half-life calculations of CAP-TAC1 in plasma. 10 UM of either synthetic CAP-TAC1 or SP was incubated with 100 μl of mouse plasma. Samples were incubated at 37° C. for 0, 10, 30, 60, or 90 minutes (N=3 for each time point). At the indicated time point, 1 μl of HALT protease inhibitor was immediately added, and plasma was boiled and prepared using the peptidomics workflow described above. LC-MS spectra were obtained using Agilent 6545 Quadrupole time-of-flight LC-MS instrument as described above. Relative peptide levels were determined by total ion count area of exact mass (±50 ppm, m/z=724.3, +1 for CAP-TAC1 and m/z=674.4, +1 for Substance P) peak that coeluted with synthetic standards. Basal plasma concentrations of Substance P and CAP-TAC1 were determined in samples with no synthetic peptide spiked in (N=3) with a standard curve comparison. Half-lives were calculated using an exponential decay fit.

Metabolic chamber studies. For acute energy expenditure studies, food intake, RER, movement, VO2, and VCO2 were collected with CLAMS Oxymax Metabolic Cages. Mice were placed individual metabolic cages for 24 hours prior to experiment. For CAP-GDF15 experiments, mice were injected with either vehicle, 50 mg/kg CAP-GDF15, or 50 mg/kg scrambled CAP-GDF15 at 5 μm at T=0. For CAP-TAC1 experiments, mice were injected with either vehicle (18:1:1 Saline: DMSO: Kolliphor EL) or 15 mg/kg CAP-TAC1 (pGlu-FFGLM-NH2) at ~12 μm (T=0). Food intake, RER, movement, VO2, and VCO2 were collected every 7 minutes for 16 hours immediately after injection. All experiments were done with 3-5 month old DIO mice (Jackson, stock 380050), fed high fat diet ad libitum.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A pharmaceutical composition comprising:
a capped peptide comprising an N-terminal pyroglutamylation modification and a C-terminal amidation modification, wherein the capped peptide is not Thyrotropin-releasing hormone (TRH) or (Gonadotropin-releasing hormone (GnRH)/luteinizing hormone-releasing hormone (LHRH); and
a pharmaceutically acceptable delivery vehicle.

2. The pharmaceutical composition according to claim 1, wherein the capped peptide comprises from 2 to 30 amino acid residues.

3. The pharmaceutical composition according to claim 2, wherein the capped peptide comprises from 3 to 25 amino acid residues.

4. The pharmaceutical composition according to any of the preceding claims, wherein the capped peptide is a murine peptide.

5. The pharmaceutical composition according to claim 4, wherein the capped peptide is a peptide listed in FIG. 14.

6. The pharmaceutical composition according to any of claims 1 to 3, wherein the capped peptide is a human peptide.

7. The pharmaceutical composition according to claim 6, wherein the capped peptide is a peptide listed in FIG. 15.

8. The pharmaceutical composition according to any of the preceding claims, wherein the capped peptide exhibits physiological activity.

9. The pharmaceutical composition according to claim 8, wherein the physiological activity is mammalian tachykinin receptor agonist activity.

10. The pharmaceutical composition according to claim 9, wherein capped peptide is CAP-TAC1 or a peptide having 80% or more identity with CAP-TAC1.

11. The pharmaceutical composition according to claim 8, wherein the physiological activity is activating metabolic activity.

12. The pharmaceutical composition according to claim 11, wherein capped peptide is CAP-TAC1 or a peptide having 80% or more identity with CAP-TAC1.

13. The pharmaceutical composition according to claim 8, wherein the physiological activity is hypophagic activity.

14. The pharmaceutical composition according to claim 13, wherein capped peptide is CAP-GDF15 or a peptide having 80% or more identity with CAP-GDF15.

15. The pharmaceutical composition according to claim 8, wherein the physiological activity is growth hormone secretagogue activity.

16. The pharmaceutical composition according to claim 15, wherein capped peptide is CAP-WNT9A or a peptide having 80% or more identity with CAP-WNT9A.

17. A method comprising administering a capped peptide to a subject in need thereof, wherein the capped peptide comprises an N-terminal pyroglutamylation modification and a C-terminal amidation modification, and is not Thyrotropin-releasing hormone (TRH) or (Gonadotropin-releasing hormone (GnRH)/luteinizing hormone-releasing hormone (LHRH).

18. The method according to claim 17, wherein the capped peptide comprises from 2 to 30 amino acid residues.

19. The method according to claim 18, wherein the capped peptide comprises from 3 to 25 amino acid residues.

20. The method according to any of claims 17 to 19 wherein the capped peptide is a murine peptide.

21. The method according to claim 20, wherein the capped peptide is a peptide listed in FIG. 14.

22. The method according to any of claims 19 to 21, wherein the capped peptide is a human peptide.

23. The method according to claim 22, wherein the capped peptide is a peptide listed in FIG. 15.

24. The method according to any of claims 17 to 23, wherein the capped peptide exhibits physiological activity.

25. The method according to claim 24, wherein the physiological activity is mammalian tachykinin receptor agonist activity.

26. The method according to claim 25, wherein capped peptide is CAP-TAC1 or a peptide having 80% or more identity with CAP-TAC1.

27. The method according to claim 24, wherein the physiological activity is activating metabolic activity.

28. The method according to claim 27, wherein capped peptide is CAP-TAC1 or a peptide having 80% or more identity with CAP-TAC1.

29. The method according to claim 24, wherein the physiological activity is hypophagic activity.

30. The method according to claim 29, wherein capped peptide is CAP-GDF15 or a peptide having 80% or more identity with CAP-GDF15.

31. The method according to claim 24, wherein the physiological activity is growth hormone secretagogue activity.

32. The method according to claim 31, wherein capped peptide is CAP-WNT9A or a peptide having 80% or more identity with CAP-WNT9A.

33. The method according to claim 25 or 26, wherein the method is a method of treating a subject for a neurological disorder.

34. The method according to claim 33, wherein the neurological disorder is depressive disorder, anxiety disorder, chronic pain, addiction, pruritus, psychosis or schizophrenia.

35. The method according to claim 25 or 26, wherein the method is a method of treating a subject for an inflammatory disorder.

36. The method according to claim 35, wherein the inflammatory disorder is irritable bowel syndrome, inflammatory bowel disease, asthma, colitis, ileus or fibrosis.

37. The method according to claim 25 or 26, wherein the method is a method of treating a subject for nausea and vomiting.

38. The method according to any of claims 27 to 30, wherein the method is a method of treating a subject for obesity or an obesity-related disorder.

39. The method according to claim 31 or 32, wherein the method is a method of treating a subject for a growth hormone deficiency or a growth hormone deficiency-related disorder.
